(19) United States
(12) Patent Application Publication
(10) Pub. No.: US 2025/0143596 A1
(43) Pub. Date: May 8, 2025

(54) GIANT MAGNETOELASTICITY ENABLED SELF-POWERED PRESSURE SENSOR FOR BIOMONITORING

(71) Applicant: The Regents of the University of California, Oakland, CA (US)

(72) Inventor: Jun CHEN, Los Angeles, CA (US)

(73) Assignee: The Regents of the University of California, Oakland, CA (US)

(21) Appl. No.: 18/694,371

(22) PCT Filed: Sep. 23, 2022
(86) PCT No.: PCT/US2022/044617

§ 371 Date: Mar. 21, 2024

Related U.S. Application Data
(60) Provisional application No. 63/247,641, filed on Sep. 23, 2021.
(60) Provisional application No. 63/357,547, filed on Jun. 30, 2022.

(51) Int. Cl. A61B 5/05; A61B 5/00; A61B 5/11; A61B 5/113; H01F 7/02

(52) U.S. Cl. CPC A61B 5/05; A61B 5/0022; A61B 5/1102; A61B 5/1126; A61B 5/113; A61B 5/6804; A61B 2562/0223; A61B 2562/18; H01F 7/02

(57) ABSTRACT

The present embodiments relate generally to a soft system for producing a giant magnetoelastic effect. In some embodiments, the soft system is composed of platinum-catalyzed silicone polymer matrix and neodymium-iron-boron nanomagnets. The soft system shows up to four times more enhancement of the magnetomechanical coupling factor (T/Pa) than traditional rigid counterparts owing to a distinct physical mechanism. In embodiments, the giant magnetoelastic effect is coupled with magnetic induction to implement a soft magnetoelastic generator (MEG) as an approach to biomechanical energy conversion, a technology that was heretofore conventionally challenged by low current, high internal impedance, and low water/humidity resistance for decent operation stability. This new method of biomechanical-to-electrical conversion is intrinsically waterproof since the magnetic fields are able to penetrate water with negligible intensity loss. Thus, it was demonstrated to work stably on wet skin or in body fluids without any encapsulation, opening up alternative avenues for practical human-body centered energy, sensing, and therapeutic applications.

### CROSS-REFERENCE TO RELATED APPLICATIONS

The present application is a National Stage Entry under 35 U.S.C. § 371 of International Application No. PCT/US2022/044617, filed Sep. 23, 2022, which claims priority to U.S. Provisional Patent Application No. 63/247,641 filed Sep. 23, 2021, and U.S. Provisional Patent Application No. 63/357,547 filed Jun. 30, 2022, the contents of all such applications being incorporated herein by reference in their entirety.

### TECHNICAL FIELD

The present embodiments relate generally to magnetoelastic effects, biomonitoring, wearable and implantable devices and soft bioelectronics.

### BACKGROUND

Magnetoelastic effect is usually observed in rigid metal alloys under an externally applied magnetic field. It would be desirable to achieve similar or superior magnetoelastic results in other materials such as soft materials.

It is against this technological backdrop that the present Applicant sought a technological solution to these and other technological issues deeply rooted in this technology.

### SUMMARY

The present embodiments relate generally to a soft system for producing a giant magnetoelastic effect. In some embodiments, the soft system is composed of platinum-catalyzed silicone polymer matrix and neodymium-iron-boron nanomagnets. The soft system shows up to four times more enhancement of the magnetomechanical coupling factor (T/Pa) than traditional rigid counterparts owing to a distinct physical mechanism. In embodiments, the giant magnetoelastic effect is coupled with magnetic induction to implement a soft magnetoelastic generator (MEG) as an approach to biomechanical energy conversion, a technology that was heretofore conventionally challenged by low current, high internal impedance, and low water/humidity resistance for decent operation stability. This new method of biomechanical-to-electrical conversion is intrinsically waterproof since the magnetic fields are able to penetrate water with negligible intensity loss. Thus, it was demonstrated to work stably on wet skin or in body fluids without any encapsulation, opening up alternative avenues for practical human-body-centered energy, sensing, and therapeutic applications.

In accordance with one or more first aspects, therefore, the present embodiments relate to the discovery of the giant magnetoelastic effect in a soft body for high-performance biomechanical-to-electrical energy conversion.

In accordance with one or more second aspects, the present embodiments relate generally to methods and apparatuses for obtaining a giant magnetoelastic effect in a 1D soft microfiber with up to 8.4 times enhancement of magnetomechanical coupling comparing to that in the traditional bulky metal alloys.

In accordance with one or more third aspects, the present embodiments couple the giant magnetoelastic effect with magnetic induction to make a self-powered biomechanical sensor with stretchability up to 550%.

In accordance with one or more fourth aspects, the present embodiments relate to a textile magnetoelastic generator (MEG) as a new mechanism for biomechanical energy harvesting.

In accordance with one or more fifth aspects, the present embodiments relate to a magnetoelastic sensor array for self-powered human-machine interface.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIGS. 1A to 1F are diagrams illustrating example aspects of creating giant magnetoelastic effect in a soft manner. A: Schematics of the internal structure of a soft system composed of NdFeB nanomagnets in a polymer matrix. B: SEM of the soft system, scale bar: 60 μm. C: Micro-CT of the soft system, scale bar: 0.4 mm. D: Illustration showing the microscale porosities subjected to a stretch deformation, improving the softness of the system. E: Magnetoelastic performance of different systems in terms of applied compressive pressure and associated relative magnetic flux density changes. F: Magnetoelastic performance of different systems in terms of piezomagnetic coefficient and an external magnetic field requirement.

FIGS. 2A to 2G illustrate aspects of an analytical model of the giant magnetoelastic effect. A-C: Magnetic flux density mappings of the soft system under 0 kPa (A), 139 kPa (B), and 278 kPa (C) compressive pressure. Scale bars: 4 mm. D: Illustration of the internal structure changes of the soft system in the initial state, the compressed state and reverse back to the initial state based on a wavy chain model. E: Original magnetic domain distribution inside the wavy chain in the initial and relaxed states. F: Varied magnetic domain inside the wavy chain in the compressed state. G: Comparison of the experimental magnetic field reduction and the wavy chain model prediction showing consistency.

FIGS. 3A to 3H illustrate aspects of a Standard evaluation of the combining effect of giant magnetomechanical coupling and magnetic induction for mechanical-to-electrical conversion. A: Soft waterproof MEG is composed of nanomagnets inside a polymer matrix as the GMMC layer and a patterned liquid metal receiver as the MI layer. B: Soft MEGs are sweatproof and can be used for energy and sensing aspects of bioelectronics. C: Optimization of the soft MEG against nanomagnet concentration: D: Mechanical properties of the soft MEG against nanomagnet concentration. E: Optimization of the soft MEG against structures of the MI layer. F: Soft MEG demonstrates ultralow internal impedance of ˜20Ω. G: Output power dependence on the electric impedance of soft MEGs with different MI layers. H: Isc comparison of different mechanisms used to harvest biomechanical energy in a wearable form.

FIGS. 4A to 4J illustrate a demonstration of the combining effect for wearable and implantable power generation. A: Scheme of the soft MEG as a wearable power generation source for small wearable electronics. B: Output current and power dependence on the electrical impedance of the soft MEG for wearable power generation. C; Voc of the soft MEG under continuous hand tapping. D: Short-current of the soft MEG under continuous hand tapping. E: Charging of 22 μF, 47 μF, and 100 μF capacitors by hand-tapping the soft MEG. F: Scheme of a soft waterproof MEG as an implantable power generation source. G: Illustration of the ultrasound energy harvesting under tissue using soft waterproof MEG. H; Isc output of the optimal MEG under 5-, 10-, and 15-mm of the porcine tissue with ultrasound excitation of different powers from 10% to 100%. I: Isc of the soft MEG in 300 us with ultrasound excitation of 100% power. J: Output current and power dependence on the electrical impedance of the soft MEG with ultrasound excitation of 100% power.

FIGS. 5A to 5G illustrate a demonstration of the combining effect for human cardiovascular system characterization. A: Schematic of the self-powered arterial pulse sensing mechanism of the soft MEG. B: Photograph showing a soft MEG in a smart watch style inside the water. C: Comparison of the arterial pulse waveforms obtained from the soft MEG under water and with sweat. D. Key arterial parameters extracted from the fine structure of the pulse waveform obtained with artificial perspiration. E: Illustration of a soft-MEG-based integrated cardiovascular health monitoring system. F: Specifically-designed APP interface for one-click health data sharing. G: Customized mobile APP interface for soft-MEG-based cardiovascular health monitoring.

FIGS. 6A and 6B are photo SEMs of the NdFeB nanomagnets and the porous GMMC layer. (A) SEM of NdFeB particles, scale bar: 20 μm. (B) SEM of the GMMC layer, scale bar: 200 μm.

FIGS. 7A to 7D are Micro-CTs showing the internal structure of a typical GMMC layer. A: 3D Micro-CT of the GMMC layer. B-D: XY, XZ and YZ section view of the GMMC layer, scale bars: 1 mm, 1 mm and 2 mm.

FIG. 8 illustrates an example Configuration of a rigid magnetoelastic system based on Galfenol or Terfenol-D. A permanent magnet or an additional coil (not shown in the figure) provides an external magnetic field to the device. When a pressure is applied, the magnetization of the alloy will change accordingly lead to a magnetic flux density change in the rigid coils. Correspondingly, a voltage and current will be excited following Faraday's Law,

FIG. 9 illustrates an example configuration of magnetoelastic effect in the iron-silicon-rubber system. A bulky electromagnet was used to provide necessary magnetic field driving the iron-silicon-rubber system. There is an excitation coil to provide the external electric input. The device contains a steel shear motion imposer to impose shear deformation in the iron-silicon-rubber system, which cause a magnetization change. The sear coil captures the magnetic flux density change of the iron-silicon-rubber system and convert it to electric output.

FIG. 10 illustrates an example experimental setup of the magnetic flux density mapping measurements.

FIG. 11 is a graph illustrating the value of 0.5×f(α)−0.1503 versus α with different n. The value of 0.5×f(α)−0.1503 is identical for all n values. Therefore, n=2,000,000 is chosen in all the calculations because it is accurate enough and timesaving.

FIGS. 12A to 12C are Photographs of the soft MEG in different deformation states. A: Conformably attached to human skin. B: Stretched state. C: Twisted state. Artificial perspiration was sprayed on the surface of the soft MEG, scale bars: 1 cm.

FIGS. 13A and 13B illustrate Control experiments showing the dominance of giant magnetoelastic effect in the soft MEG. A: The current pulse generated by pressing rigid NdFeB magnet on a MI layer with soft copper cycles. B: The current pulse generated by pressing a GMMC layer on the same MI layer, Both the rigid NdFeB magnet and the GMMC layer have the same magnetic pole direction as shown in the inset schemes. When the rigid NdFeB magnet is pressed on the MI layer, the relative distance between the magnet and the MI layer decrease leading to an increase of magnetic flux density in the MI layer and induced a current pulse in FIG. 13A. When the same pressure is applied to the GMMC layer, there are two processes happening. One is the same as the rigid magnet with an increase of magnetic flux density. Another is due to the giant magnetoelastic effect of the GMMC layer and cause a decrease of magnetic flux density. The current pulse generated when pressing the GMMC layer is completely reversed compared to pressing the rigid NdFeB magnet, which is due to the decrease of the magnetic flux density through the MI layer and clearly indicates the dominance of the giant magnetoelastic effect in the soft MEG system.

FIG. 14A to 14E are graphs illustrating Magnetic flux density mappings of GMMC layers with different NdFeB concentrations. A: 50 wt % B: 67 wt % C; 75 wt % D: 80 wt % E: 83 wt % NdFeB concentrations. Blue represents the positive side with south pole and red represents the negative side with north pole.

FIGS. 15A and 15B illustrate Magnetic and mechanical properties of the GMMC layers. A: Relationship of the NdFeB nanomagnet concentration and the magnetic flux density (middle of surface). B: Stress strain curves of the GMMC layers with different NdFeB nanomagnet concentrations. With increasing weight concentration of NdFeB nanomagnets, the in magnetic flux density (north pole, middle of surface) and Young's modulus of the soft system increased from 7.9 to 54.2 mT and 177.99 to 692.23 kPa, respectively, while its fracture strain decreased from 432.48% to 189.28%. Nevertheless, the soft system still exhibits comparable mechanical softness to human skin and tissue (15, 16) at 83 wt % with a surface magnetic flux density of around 50 mT.

FIGS. 16A to 16H illustrate example Shapes of liquid metal coils in multilayered MI layers. A: 11 cycles in the first and third layers. B: 8 cycles. C: 5 cycles. D: 2 cycles. E: 11 cycles in the second layers. F: 8 layers in the first layer for the cardiovascular health management. G: 8 layers in the second layer for the cardiovascular health management. H: Overall multilayer structure of the MI layer. The scale for FIGS. 16A to 16E is the same and the scale for FIGS. 16F and 16G is the same.

FIGS. 17A and 17B illustrate Current and voltage dependence on the electric impedance of the soft MEGs (A) 2-layer-structured MI layer with liquid metal cycles. (B) 3-layer-structured MI layer with liquid metal cycles.

FIGS. 18A to 18C illustrate Stability of the soft MEG. (A) Normalized voltage output of the soft MEG during 10,000 compress-relax cycles at 20 Hz. (B) Normalized voltage output of the soft MEG during the first 10 compress-relax cycles. (C) Normalized voltage output of the soft MEG during the last 10 compress-relax cycles. The MEG has a 2-layer-structued MI layer with liquid metal cycles.

FIGS. 19A to 19F illustrate aspects of Characterization of a soft MEG under large-angle bending deformations. (A) Schematic of the MEG in relax state. (B) Schematic of the MEG under bending deformation. (C) Isc output of the MEG under 5 consecutive bending deformations. (D) Waveform of the Isc signal under bending deformation. (E) Open-circuit voltage (Voc) output of the MEG under 5 consecutive bending deformations. (F) Waveform of the Voc signal under bending deformation.

FIGS. 20A to 20F illustrate aspects of Characterization of a soft MEG under large-angle twisting deformations. (A) Schematic of the MEG in relax state. (B) Schematic of the MEG under twisting deformation. (C) Isc output of the MEG under 5 consecutive twisting deformations. (D) Waveform of the Isc signal under twisting deformation. (E) Voc output of the MEG under 5 consecutive twisting deformations. (F) Waveform of the Voc signal under twisting deformation.

FIGS. 21A to 21F illustrate aspects of Characterization of a soft MEG under stretching deformations. (A) Schematic of the MEG in relax state. (B) Schematic of the MEG under stretching deformation. (C) Isc output of the MEG under 5 consecutive stretching deformations. (D) Waveform of the Isc signal under stretching deformation. (E) Voc output of the MEG under 5 consecutive stretching deformations. (F) Waveform of the Voc signal under stretching deformation.

FIG. 22 illustrates Voltage dependence on the electric impedance of wearable soft MEG power generator.

FIG. 23 illustrates the equivalent circuit to boost the voltage and rectify the electric output of the soft MEG.

FIG. 24 is a photograph of the soft MEG driving a commercial thermometer. Scale bar: 2 cm.

FIG. 25 is an exploded view of the soft MEG ultrasound harvester under porcine skin and tissue.

FIG. 26 is a Photograph of the porcine skin and tissue used in the ex vivo experiments.

FIGS. 27A and 27B illustrate aspects of Isc of the soft MEG without and with a GMMC layer implanted 5 mm under the tissue. (A) Without and (B) with an 800 μm thick GMMC layer composed of 83 wt % NdFeB particles. The ultrasound power is 75%.

FIGS. 28A and 28B illustrate aspects of Isc of the soft MEG without and with a spacer implanted 5 mm under the tissue. (A) Without and (B) with the polystyrene spacer. The ultrasound power is 75%. The GMMC layer is 800 μm thick and 83 wt % of 5 μm NdFeB particles.

FIGS. 29A to 29E illustrate aspects of Isc output of the soft MEG implanted 5 mm under tissue with GMMC layers. (A) 400 μm thick, 83 wt % 5 μm NdFeB particles. (B) 400 μm thick, 83 wt % 25 μm NdFeB particles. (C) 800 μm thick, 83 wt % 5 μm NdFeB particles. (D) 800 μm thick, 83 wt % 25 μm NdFeB particles. (E) 1800 μm thick, 83 wt % 5 μm NdFeB particles.

FIGS. 30A to 30E illustrate aspects of Isc output of the soft MEG implanted 5 mm under tissue with different ultrasound power. (A) 10% (B) 25% (C) 50% (D) 75% (E) 100% ultrasound power. The GMMC layer used is 1800 μm thick and composed of 83 wt % 5 μm NdFeB microparticles.

FIGS. 31A to 31E illustrate aspects of Isc output of the soft MEG implanted 10 mm under tissue with different ultrasound power. (A) 10% (B) 25% (C) 50% (D) 75% (E) 100% ultrasound power. The GMMC layer used is 1800 μm thick and composed of 83 wt % 5 μm NdFeB microparticles.

FIGS. 32A to 32E illustrate aspects of Isc output of the soft MEG implanted 15 mm under tissue with different ultrasound power. (A) 10% (B) 25% (C) 50% (D) 75% (E) 100% ultrasound power. The GMMC layer used is 1800 μm thick and composed of 83 wt % 5 μm NdFeB microparticles.

FIGS. 33A and 33B illustrate Electric output of a GMMC layer before and after submerged in artificial perspiration. (A) Isc. (B) Voc.

FIG. 34 illustrates A typical contour of an arterial pulse wave with fine structure measured by a soft MEG.

FIG. 35 illustrates example Heart rate extracted from the soft MEG under water without an encapsulation layer.

FIGS. 36A to 36D illustrate example cardiovascular parameters extracted from the soft MEG in a sweaty condition. (A) Pulse wave velocity and K value. (B) Upstroke time and stiffness index. (C) Augmentation index and left ventricular ejection time. (D) Standard deviation of heartbeat intervals.

FIG. 37 is an example System level block diagram of the self-designed cardiovascular health monitoring system. It is composed of the analog front (orange), micro-control unit (carneous), and digital signal processor (blue).

FIGS. 38A to 38D illustrate aspects of APP interfaces of customized health monitoring system for data demonstration. The APP includes Bluetooth connection, pulse wave monitoring, cardiovascular health parameter management and data sharing. (A) User interface of the APP's icon. (B) APP interface showing the Bluetooth is successfully connected to the device. (C) APP interface showing the heart rate, pulse waveform, K value, pulse wave velocity and stiffness index of a young adult user. (D) APP interface showing the specially designed health table with 8 key cardiovascular parameters derived from the pulse wave of a young adult user.

FIG. 39A to 39D illustrate example APP interfaces of customized health monitoring system for data sharing. (A) APP interface showing the options of health data sharing capability through email, Bluetooth, or messaging. (B,C) APP interface showing the capability of saving health data to cloud for afterwards usage. (D) APP interface showing the health data sent to a professional physician through email.

FIGS. 40a-40l illustrate example aspects of Discovering the giant magnetoelastic effect in soft microfibers according to embodiments. a, Schematics of the fabrication process of the soft magnetic microfibers. b, Micro-CT image and c, Schematics of the soft magnetic microfiber. Scale bar: 0.4 mm. d, Stress strain curves of the soft magnetic microfibers with 83 wt % magnetic concentration. e, f, Magnetic flux density mapping of the soft magnetic microfiber on south pole surface in the original state (e) and under a compressed pressure 300 kPa (f). Scale bar: 0.24 mm. g, Magnetic field variation of the soft magnetic microfiber under applied stress on the south pole surface. h, Magnetic hysteresis loop of the soft magnetic microfiber in original state and in compressed state. i, j, Schematics of the magnetic dipole distribution in soft magnetic microfiber of initial state (i) and compressed state (j), k, Schematics of the wavy chain model. l, Performance comparison of the soft magnetic microfibers with rigid metal alloys.

FIGS. 41a to 41l illustrate example aspects of Designing an origami-programmed textile MEG according to embodiments. a, Schematic design of the textile MEG. b, Schematics of the working mechanism of the textile MEG. c, Photograph showing the weaving processing with a loom. Scale bar: 1 cm. d-g, Schematics of parallel folded magnetic microfiber (d), straight magnetic microfiber (e), perpendicular folded magnetic microfiber (f), and annular folded magnetic microfiber (g) with an applied magnetic field. h, Schematics of textile MEGs with five origami-programming patterns. i, Dependence of the short-circuit current (Isc) and open-circuit voltage (Voc) of the textile MEGs on different origami patterns. j-l, Dependence of the electrical output of the textile MEGs on the cross-section area of the magnetic microfibers (j), number of the magnetic microfibers (k), and number of the conductive yarns (l). Error bars are standard deviations of the results from five samples.

FIGS. 42a to 42i illustrate example aspects of Intrinsically waterproof textile for wearable power generation. a, b, Schematics showing the three different weaving patterns (a) and the corresponding measured electrical signals (b) of the textile MEG. Error bars are standard deviations of the results from five samples. c, d, Schematics showing four excitation modes of the textile MEG (c) and the corresponding measured electrical signals (d). e, Dependence of the output voltage and current of the textile MEG on the external load resistances. f, Dependence of the power on the external load resistances. g. A comparison of the power generation with the current textile biomechanical energy harvesting technologies. h, Charging commercial capacitors up to 3 V. i, Photograph of the textile MEG mixed with wool fibers sustainably driving a wearable biosensor system. Scale bar; 2 cm.

FIGS. 43a to 43k illustrate example aspects of Measuring cardiovascular parameters underwater without encapsulation for telehealth. a, Photograph of the textile wristband as a wearable pulse sensor. Scale bar: 2 cm. b, Schematics of the working principle of the textile wristband. c, Sensitivity of the textile MEG with respect to applied pressure at a frequency of 1 Hz. d, Measured pulse wave profile in one cardiac cycle when the encapsulation-free textile wristband was tested with perspiration and under water. The shadow areas indicate the standard deviation of the results from five pulse wave profile. e, Characteristic K value and PWV obtained from pulse wave signals. f, Measured pulse wave profiles when textile wristband was soaked in artificial perspiration and water for up to 168 hours. g, A water vapor transmission rate test showing the perspiration transmission through the textile MEG. h, Photograph showing the skin irritation results of the permeable textile MEG and an impermeable magnetic film on the forearm. Scale bar: 1.5 cm. i, The developed user interface on a cellphone APP. j, Schematics of a fully integrated CMS. The personal heath data could be processed via two pathways: one is directly sending to the physicians for immediate clinical diagnosis, and the other is uploading into the cloud to conduct big data analysis for data-driven diagnosis. k, Screenshot of cellphone user interface showing the personal health data sharing options.

FIGS. 44a to 44g illustrate example aspects of Characterization of the soft magnetic fibers via Micro-CT image. a, 3D Micro-CT image showing the whole view of the magnetic microfiber. Scale bar: 1.5 mm. b, Schematics and c, Micro-CT image showing the XZ. section view of the magnetic microfiber. Scale bar: 1.5 mm. d, Schematics and e, Micro-CT image showing the XY section view of the magnetic microfiber. Scale bar: 2 mm. f, Schematics and g, Micro-CT image showing the YZ section view of the magnetic microfiber. Scale bar: 2 mm.

FIGS. 45a to 45d illustrate example Stress strain curves of the soft magnetic fibers. a, The photography of the soft magnetic fibers a, in the initial state. b, in the stretching state. Scale bars: 1 cm. c, Stress strain curves of the soft magnetic fibers with 75 wt % and 50 wt % magnetic concentrations. d, Magnetic hysteresis loop of the soft magnetic microfibers with 83 wt %, 75 wt % and 50 wt % magnetic concentrations.

FIGS. 46a to 46c illustrate example aspects of Massive production of the soft magnetic fibers and conductive yarns. a, Photograph of three spools of magnetic microfibers. Scale bar: 1.4 cm. b, Schematics showing multiaxial yarn winding machine. c, Photograph of three spools of conductive yarns. Scale bar: 1.3 cm.

FIGS. 47a to 47e illustrate example aspects of Investigation of the magnetic flux density variation of the soft magnetic fiber in response to the applied compressive pressure. a, Schematics showing three-axial motion platform, b, Magnetic field variation of the soft magnetic fiber under applied stress on the side surface. c, Magnetic field reduction of the soft magnetic fiber under applied stress on side surface. d, Magnetic field reduction of the soft magnetic fibers with 83 wt % of SrFe12O9 or Fe3O4 under applied stress. e, Comparison of the experimental magnetic field variation and the wavy chain model prediction showing consistency.

FIGS. 48a to 48c illustrate example aspects of Measuring cardiovascular parameters with the textile wristband. a, Sensitivity of textile MEG with respect to frequency at a fixed pressure of 4.6 kPa. b, Characteristic SI, UT obtained from pulse wave profiles. c, A loading and unloading test were performed for 20,000 times

FIG. 49 illustrates an example System-level block diagram of the wireless wearable CMS according to embodiments.

FIGS. 50a and 50b illustrate example Scanning electron microscope (SEM) images of a cross-section view of a soft magnetic microfiber showing the nanomagnets were evenly dispersed. a, Scale bar: 100 μm. b, Scale bar, 20 μm.

FIG. 51 illustrates example Schematics of the soft magnetic microfibers showing magnetic dipoles were aligned in the soft polymer matrix.

FIGS. 52a to 52c illustrate an example Magnetic flux density mapping of the soft magnetic fiber. a-c, Magnetic flux density mapping of the soft magnetic microfiber on north pole surface in the original state (a), under a compressed pressure 0 kPa (b) 180 kPa and (c) 300 kPa. Scale bar: 0.24 mm.

FIGS. 53a to 53c illustrate an example Magnetic flux density mapping of the soft magnetic fiber, a-c, Magnetic flux density mapping of the soft magnetic microfiber on side surface in the original state (a), under a compressed pressure 0 kPa (b) 40 kPa and (c) 86 kPa. Scale bar: 0.24 mm.

FIG. 54 illustrates an example SEM image of the silver-coated nylon microfibers. Scale bar: 4 μm.

FIGS. 55a to 55c illustrate example Schematics showing three different weaving patterns. a, Plain. b, Stain. c, Twill.

FIG. 56 illustrates example aspects of Electrical output of the textile MEG under continuous hand tapping.

FIGS. 57a and 57b illustrate example aspects of The schematics and electrical performance of textile based on triboelectric effect. a, Schematics of textile based on triboelectric effect. b, Voltage output of textile based on triboelectric effect testing in air and in water condition.

FIGS. 58a and 58b illustrate example Photographs of the ring-shaped textile wristband. a, Textile wristband tested under water. Scale bar: 1.5 cm. b, Textile wristband tested with artificial perspiration condition. Scale bar: 1.5 cm.

FIG. 59 is a graph illustrating example aspects of Magnetic hysteresis loop of the NdFeB nanomagnets,

FIGS. 60a to 60d illustrate example Photographs of the artificial perspiration droplet absorbed by textile MEG. (a) 0 ms (b) 388 ms (c) 550 ms and (d) 660 ms. Scale bars: 0.5 mm.

FIG. 61 illustrates an example Screenshot of the cellphone App showing a health table as a user interface.

FIGS. 62a and 62b illustrate example Screenshots of the cellphone App showing the sharing options with the physicians over a distance. a, Screenshot of the cellphone App showing the sharing options. b, Screenshot of the cellphone App showing the health data is sent to the physicians by email.

FIGS. 63a and 63b illustrate example Screenshots of the cellphone App showing the health data is uploaded into the cloud, which could be download with authorized access.

FIG. 64 illustrates example Schematics of the 3D MEG textiles. The Schematics consists of multiple-layer weaved magnetic microfibers and conductive yarns stacking together.

FIG. 65 is a graph illustrating an example pulse wave profile in one cardiac cycle obtaining from an textile wristband according to embodiments.

FIG. 66 illustrates example Schematics of the weaving procedure of constructing the origami-programmed textile magnetoelastic generator.

FIGS. 67A to 67K illustrate example aspects of a Giant magnetoelasti magnetoelastic effect in a soft polymer matrix according to embodiments. (A) Schematic of the internal structure of a soft polymer matrix. (B) Schematic of the soft polymer matrix showing magnetic dipoles aligned in the soft polymer matrix. (C) Stress-strain curves of the soft polymer matrix with 75 wt % and 50 wt % magnetic concentrations. (D) The surface magnetic field distribution of the soft polymer composites. (E-F) Magnetic flux density mappings of the soft system in original state and compressed state. Scale bars: 0.24 mm. (G) Magnetic field variation of the soft polymer matrix under pressure from 0-2,081 kPa. (H) Magnetic hysteresis loop of the soft polymer matrix in original state and compressed state. (I) Schematics of the wavy chain model. (J-K) Schematic of the magnetic dipole distribution in soft magnetic microfiber of the initial state (J) and compressed state (K).

FIGS. 68A to 68M illustrate an example Magnetelastic sensor performance characterization according to embodiments. (A) Schematic setup of the thermal fiber drawing tower. (B) Photograph of one spool of liquid metal microfibers. Scale bar: 2 mm. (C) Strain-stress curves of the liquid metal microfibers and the hysteresis loops with increasing strains up to 700%. (D) Cycling tensile test at a fixed strain of 200% for 100 cycles. (E) Diagram of the magnetelastic sensor. (F) SEM image of the cross-section of the liquid metal microfibers. Scale bar: 100 μm. (G) 3D Micro-CT image showing the whole view of the as-fabricated magnetelastic sensor. Scale bar: 2 mm. (H) Strain response curve for six consecutive loading-unloading cycles. (I) Pressure response curve for six consecutive loading-unloading cycles. (J) Current output of the magnetelastic sensor responding to the pressure of 3.5 Pa. Inset: Picture of an magnetelastic sensor. Scale bar: 1 cm. (K) Sensitivity of the magnetelastic sensor with respect to applied pressure at a frequency of 1 Hz. Inset: Picture of an magnetelastic sensor. Scale bar: 0.5 cm. (L) Current output of the magnetelastic sensor responding to the pressure of 3 kPa under loading of 0 Pa, 300 kPa, 600 kPa, and 2 MPa. (M) The response time of the magnetelastic sensor.

FIGS. 69A to 69G illustrate example aspects of Magnetelastic sensors for wearable biomonitoring according to embodiments. (A) The working mechanism of the magnetelastic sensor. (B) Current output associated with the arterial pulse taken from the wrist. (C) Current output associated with couging. (D) Current output associated with finger bending at different bending angles including, 30°, 60°, and 90° from left to right on the graph, respectively. (E) Current output associated with deep breathing, on the left, and rapid breathing, on the right. (F) Current output associated with the bending of the knee. (G) Current output associated with the movement correlated to both running and jumping on the left and the right on the graph, respectively.

FIGS. 70A to 70L illustrate example aspects of Magnetelastic sensors for implantable biomonitoring according to embodiments. (A-C) Live/Dead assay of human fibroblasts being cultured on sensor materials for 24 hours. (A) Negative control sample with cells being treated with 20% DMSO. (B) Positive control sample with cells being seeded in a cell culture dish. (C) Cells on the surface of the magnetelastic sensor. Scale bars: 200 μm. (D) Prestoblue assay detects the cell viability of fibroblasts in different experimental groups. (E-F) Scheme of the magnetelastic sensor as an implantable sensor on a porcine heart. (G) The photo showing magnetelastic sensor was placed on a porcine heart. Scale bar: 1 cm. (H) The current output of the implantable sensor on a porcine heart. (1) The current output showing different volume of fluid (60 ml, 80 ml, and 110 ml) pumed into the heart chamber. (J) The generated electric charges during each cycle to characterize the stroke volume. (K) Long-term cyclic test of the magnetelastic sensor with only a 0.01% decay. (L) Performance comparison of the magnetelastic sensor with other types of pressure sensors.

FIGS. 71A and 71B are Scanning electron microscope (SEM) images of a cross-sectional view of the soft polymer matrix showing the evenly-dispersed nanomagnets. (A) Scale bar: 30 μm. (B) Scale bar, 10 μm.

FIGS. 72A to 72F illustrate example aspects of Characterization of the soft polymer matrix via Micro-CT image. (A), Schematics showing soft polymer matrix. (B) Micro-CT image of soft polymer matrix showing NdFeB microparticles distributed uniformly in the elastomer matrix. Scale bar, 1 mm. (C-D), Schematics showing soft polymer matrix was dissected in cross-sectional top view (D) and the corresponding Micro-CT image in this view, Scale bar, 1 mm. (E-F), Schematics showing soft polymer matrix was dissected in cross-sectional front view (F) and the corresponding Micro-CT image in this view. Scale bar, 1 mm.

FIG. 73 is a Schematic showing three-axial motion platform.

FIG. 74 is a graph illustrating a Repeatable tensile test of the liquid metal microfibers from 100% to 700%.

FIG. 75 is a graph illustrating example Electric output of the magnetelastic sensor detected a drop of water from different Height

FIG. 76 is a graph illustrating aspects of the magnetelastic sensor exhibiting high-response characteristics for high-frequency vibrations up to 1 kHz.

FIGS. 77A to 77F illustrate aspects of characterization of the magnetelastic sensor under different deformations. (A) Schematic of the magnetelastic sensor under tapping deformation. Scale bar: 1 cm. (B) Electric output of the magnetelastic sensor under consecutive tapping deformations. (C) Schematic of the magnetelastic sensor under stretching deformation. Scale bar: 1 cm. (D) Electric output of the sensor under consecutive stretching deformations. (E) Schematic of the magnetelastic sensor under bending deformation. Scale bar: 1 cm. (F) Electric output of the magnetelastic sensor under consecutive bending deformations.

FIG. 78 is a graph illustrating aspects of an example magnetelastic sensor used for voice detection.

FIG. 79 is a graph illustrating Current output induced by the human frown.

FIG. 80 is a graph illustrating aspects of a Corrosion resistance test of the magnetelastic sensor,

FIG. 81 is a graph illustrating the current output showing the abnormal heart rhythms.

FIG. 82 is a block diagram of an example sensitivity measurement setup.

FIG. 83 illustrates a configuration of an example iron-silicone-rubber-system, which needs an external magnetic field to show the magnetoelastic effect.

FIGS. 84A to 841 illustrate aspects of discovering the giant magnetoelasticity in soft matter: (A) 3D Micro-CT of the soft matter and schematics of the internal structure consisted of micromagnets and polymer matrix. Scale bar, 2 cm. (B) Hysteresis loop of the soft magnetoelastic film. (C) Stress-strain curves of the soft magnetoelastic film with different micromagnet concentrations. (D) Magnetic flux density mappings of the soft magnetoelastic film with/without compression. Scale bar, 0.5 cm. (E) Magnetoelastic performance of different micromagnet concentrations in terms of applied compressive stress and associated magnetic flux density changes. (F) Performance comparison of different magnetoelastic systems in terms of the magnetomechanical coupling factor d33. (G) Wavy chain model to explain the giant magnetoelasticity in soft matter. The dominant parameters are the horizontal center-to-center micromagnet distance h and the vertical center-to-center micromagnet distance b. (H) SEM images of the soft magnetoelastic film with marked chain structures. Scale bar, 100 μm. (I) Comparison of the experimental magnetic flux density variation under the applied pressure and the wavy chain model result shows consistency.

FIGS. 85A to 85H illustrate aspects of Utilizing the giant magnetoelasticity effect in soft matter for electricity generation. (A) Textile MEG is composed of a soft magnetoelastic film, a textile coil, and a textile substrate. (B) Photograph of the scalable textile coil. Scale bar, 2 cm. (C) SEM of the textile coil. Scale bar, 100 μm. (D) Illustration of the waterproof ability of textile MEGs. (E) Textile MEGs are sweatproof and can be used for a wide range of on-body applications. (F), (G), and (H) Dependence of the electric outputs of the textile MEGs against the textile coil size (F), distance (G), and the number of turns (H).

FIGS. 86A to 861 illustrate aspects of Textile MEGs for wearable biomechanical energy conversion. (A) Pervasive biomechanical energy sources on the human body. (B) Schematics showing three mechanical excitation modes on the textile MEGs and the corresponding measured voltage and current output. (C) Voc of the textile MEG under continuous hand tapping. (D) Isc of the textile MEG under continuous hand tapping. (E) Waterproof ability of textile MEGs in aspect of electricity generation. (F) Output current and voltage dependence on the electrical impedance of the textile MEG. (G) Output power dependence on the electrical impedance of the textile MEG. (H) Charging of 22 μF, 47 μF, and 100 μF capacitors by hand-tapping the textile MEG. (I) Photograph of the textile MEG for powering a commercial thermometer.

FIGS. 87A to 87F illustrate aspects of Standard evaluation of textile MEGs for self-powered sensing. (A) Comparison of the sensing signals obtained from the textile MEG under wet and the dry situation. (B) Sensing signal dependence on the applied pressure frequency and amplitude. (C) Dependence of the response time and signal-to-noise ratio of the textile MEGs on the applied pressure frequency. Error bars are standard deviations of the results from three samples. (D) Waveforms of the sensing signals under different applied pressure frequencies. (E) A tiny white flower, a green leaf, and a yellow flower were gently dropped onto the textile MEG and generated distinct current signals. (F) Cyclic test of the textile MEGs for more than 5000 cycles, and the enlarged view of the marked region, showing excellent stability and repeatability.

FIGS. 88A to 88G illustrate aspects of Machine learning assisted textile MEG for wearable respiratory monitoring. (A) Textile MEGs integrated on the nursing scrubs for respiratory monitoring. A customized cellphone application was developed for data display, storage, and sharing. (B) Three different kinds of respiratory patterns: normal breathing, rapid breathing, and coughing were monitored by the textile MEG. (C), (D), and (E) Machine learning algorithm to classify different respiratory activities, including the data preprocessing (C), feature extraction (D), and model training (E). (F) Classification precision of different respiratory activities. (G) Customized cellphone application interface to display the sensing results in the front-ends.

FIG. 89 is a Schematic illustration of the magnetoelasticity effect. Magnetoelastic effect is defined as the change of any magnetic property in certain materials under mechanical deformation.

FIG. 90 illustrates a Conventional magnetoelasticity system based on the rigid Galfenol or Terfenol-D for mechanical-to electrical conversion. Rigid magnetoelastic materials such as the Galfenol or Terfenol-D can be deformed by external pressure and generate the magnetization variation. The corresponding magnetic flux density change will induce an electromotive force in the rigid coils based on Faraday's law of induction.

FIGS. 91A and 91B are SEM pictures of the soft magnetoelastic film. (A) and (B) SEM pictures of the soft magnetoelastic film. These micromagnets are evenly distributed in the porous polymer matrix. Scale bars, 100 μm.

FIGS. 92A and 92B are graphs showing Size and inter-particle distribution of the micromagnets in the polymer matrix. (A) These micromagnets have a mean diameter of 53.7 μm with a standard deviation of 18.8 μm. (B) These micromagnets have a mean interparticle distance of 139.6 μm with a standard deviation of 34.0 μm.

FIGS. 93A to 93D are graphs illustrating aspects of Calculating the Young's modulus of the soft magnetoelastic film with different micromagnet concentrations by using Neo-Hookean model. (A) Calculating the Young's modulus of the soft magnetoelastic film with 40 wt % micromagnet concentrations. (B) Calculating the Young's modulus of the soft magnetoelastic film with 60 wt % micromagnet concentrations. (C) Calculating the Young's modulus of the soft magnetoelastic film with 80 wt % micromagnet concentrations. (D) Young's modulus of the soft magnetoelastic film with different micromagnet concentrations.

FIGS. 94A and 94B illustrate aspects of Working mechanisms of the wearable electromagnetic generators. Conventional electromagnetic generators require the magnets moving relative to coils, such as the horizontal movement (A) and the vertical movement (B), thus generating a motional electromotive force based on Faraday's law of induction.

FIGS. 95A to 95D illustrate aspects of Dependence of the magnetic field variation on the area ratio of the coil and the soft magnetoelastic film. (A) and (B) A smaller coil (Scoil/Sfilm<1) cannot collect all magnetic flux variation of the compressed the soft magnetoelastic film. (C) Approximately same size of the coil and the soft magnetoelastic film is the optimized ratio to take use of the magnetic field variation. (D) A large coil (Scoil/Sfilm>1) consists of adverse magnetic flux canceling each other out.

FIG. 96 is a graph illustrating Magnetic fields distribution of the magnets MATLAB stimulation demonstrates that the density of the magnetic flux decreases with the distance to the magnets. Thus, textile coil closely attached to the soft magnetoelastic film could well take use of the magnetic flux variation and generate the maximum electric outputs.

FIG. 97 is a Photograph of the textile MEG conformably attached to the human skin. This textile MEG can be conformably attached to the human skin owing to its high flexibility. Scale bar, 1 cm.

FIGS. 98A to 98C illustrate aspects of The soft magnetoelastic films under the compressed, bent, and twisted state. (A) Vertically applied pressure on the compressed soft magnetoelastic film could strongly decrease the magnetic flux density. (B) and (C) The bent (B) and twisted (C) soft magnetoelastic film would partially cancel the magnetic field variation, because some flux demonstrates the opposite direction.

FIG. 99 is an example Circuit diagram of the booster and rectifier. The equivalent circuit to boost the voltage and rectify the alternative electric output of the textile MEG into the direct output.

FIG. 100 is an example Schematic illustration of the experimental setup for sensing performance characterization. A testing system containing a function generator, power amplifier, electrodynamic shaker, pressure gauge, programmable electrometer, and computer.

FIGS. 101A to 101D are graphs illustrating aspects of Response time of the textile MEG. External pressure excitation with the input frequency of (A) 0.5 Hz, (B) 1.0 Hz, (C) 1.5 Hz, (D) 2.0 Hz were applied on the textile MEG. The response time of the textile MEG is 66 ms, 42 ms, 19 ms, and 15 ms, respectively.

FIG. 102 is a graph illustrating Signal-noise-ratio (SNR) measurement of the textile MEG. The SNR of the textile MEGs is calculated by 20×1 g (Isignal/Inoise). The average noise of the present measurement system is 2.78×10-4 mA,

FIG. 103 is a graph illustrating The stability test of the textile MEG. An amplitude-fixed pressure with a frequency of 2 Hz was applied to the textile MEG for 5,000 loading-unloading cycles.

FIGS. 104A and 104B are graphs illustrating a sweatproof ability test of the textile MEG. (A) and (B) Comparison of the respiratory signal waveforms obtained from the textile MEG under dry situation (A) and with artificial perspiration (B).

FIGS. 105A to 105C are screenshots illustrating an example Customized cellphone APP for respiratory monitoring data display. (A) APP's icon on a cellphone desktop. (B) and (C) APP interfaces showing the breaths per minute (BPM), respiratory rate (RR), cough, temperature, and test results of a young adult user under different status.

FIGS. 106A to 106C are screenshots illustrating an example Customized cellphone APP for respiratory monitoring data storage and sharing. (A) Customized cellphone APP can save the respiratory monitoring data to the cloud for further analysis. (B) Customized cellphone APP can share the respiratory monitoring data through email, Bluetooth, or messaging. (C) APP interface showing the personalized respiratory monitoring data sent to a physician through email.

FIGS. 107(a) to 107(g) illustrate an embodiment of a multifunctional magnetoelastic sensor array for touch sensing combining the effect of the magnetoelastic effect and electromagnetic induction for biomechanical-to-electrical conversion. (a) Schematic of the magnetoelastic sensor array, composed of four magnetoelastic sensors, whose components are patterned liquid metal printed onto a soft magnetoelastic film. Illustrations of magnetic alignment changing the magnetic flux density of a magnetoelastic sensor in the (b) original state and the (c) compressed state, based on the wavy chain analytical model. (d) 3D micro-CT of the liquid metal MI layer and the soft MC layer. Scale bar: 1.5 cm. Magnetic flux density mappings of a single magnetoelastic sensor (e) with and (f) without compression. Scale bars: 1.5 cm. (g) Flexible, stretchable, and waterproof magnetoelastic sensor array that can adapt to various deformations in the (i) original state, (ii) rolling state, (iii) folding state, and (iv) stretching state. Scale bars: 5 mm.

FIGS. 108(a) to 108(f) illustrate example aspects of Characterization of magnetic and mechanical properties of the magnetoelastic sensor array for biomechanical-to-magnetic conversion. (a) Magnetic field variation of the magnetoelastic sensor at different thicknesses under applied stress. (b) Stress-strain curves of the magnetoelastic sensor with different magnetic concentrations. (c) Magnetic field variation of the magnetoelastic sensor at different concentrations of micromagnets under applied stress. (d) Comparison of the Young's modulus and the initial magnetic field strength of the magnetoelastic sensor (10 mm×10 mm×1.5 mm) with different micromagnet concentrations. (e) Magnetic field variation (in a vertically upward direction) of the magnetoelastic sensor under different magnetization direction angles. Set the north as the positive direction. (f) Set-up of the magnetization orientation of the magnetoelastic sensor.

FIGS. 109(a) to 109(f) illustrate example aspects of Characterization of electrical properties of the magnetoelastic sensor array for magnetic-to-electrical conversion. (a) Drawing of the magnetoelastic sensor array with scalable dimension. (b) Dependence of the electric outputs of one magnetoelastic sensor reflecting the number of turns of the coils. (c) Generated current waveforms of the magnetoelastic sensor under various applied frequencies. (d) Dependence of the response time and SNR of the magnetoelastic sensor on the applied pressure frequency. (e) Sensitivity of the magnetoelastic sensor with respect to applied pressure at a frequency of 2 Hz. (f) Cyclic test of the magnetoelastic sensor underwater for more than 10,000 cycles.

FIGS. 110(a) to 110(d) illustrate an example Demonstration of magnetoelastic sensor array in applications of human-machine interface. (a) Photograph of the magnetoelastic sensor array which is conformal to human skin and can function even under the exposure to liquid. (b) Waterproof ability of the magnetoelastic sensor array with respect to electricity generation. (c) Recorded output signals from touch sensing of the magnetoelastic sensor array to interact with a music speaker's command components: play, pause, next, and previous. (d) Circuit design and the process flow of the acquired data from magnetoelastic sensor array, including an amplifier, the low-pass filters, two micro-controllers, two Bluetooth modules, and a relay. (e) Recorded output signals from touch sensing of the magnetoelastic sensor array after being processed by a relay.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

### Section A—Giant Magnetoelastic Effect in Soft Systems

### A. Background

In nature, under an applied magnetic field, metal alloys such as TbₓDy₁₋ₓFe₂ (Terfenol-D) and GaₓFe₁₋ₓ (Galfenol) with magnetoelastic effect could alter their inner magnetization in response to external mechanical stress and perform mechanical-to-electrical conversion through a pickup coil in a bulky and rigid manner (W. J. Fleming, New Automotive Sensors—A Review. IEEE Sens. J. 8, 1900-1921 (2008); S. Eem, H. Jung, J. Koo, Application of MR elastomers for improving seismic protection of base-isolated structures. IEEE Trans. Magn. 47, 2901-2904 (2011); Z, Deng, M. J. Dapino, Review of magnetostrictive materials for structural vibration control. Smart Mater. Struct. 27, 113001 (2018).). The optimal conversion efficiency can be only obtained with the applied stress in the order of several mega pascals As a result, it is widely employed in civil engineering for building vibration control (G. Ausanio, A. C. Barone, C. Hison, V. Iannotti, G. Mannara, L. Lanotte, Magnetoelastic sensor application in civil buildings monitoring. Sens. Actuator A Phys. 123-124, 290-295 (2005).). However, magnetoelastic effect has been ignored in the increasingly important field of bioelectronics for the following reasons: the magnetization variation in the biomechanical stress range is limited; the requirement of external magnetic field induces structural complexity, and there exists a gigantic mismatch of mechanical modulus (6 orders difference) between magnetic alloy and human tissue (Q. Su, J. Morillo, Y. Wen, M. Wuttig, Young's modulus of amorphous Terfenol-D thin films. J. Appl. Phys. 80, 3604-3606 (1996); S. Datta, J. Atulasimha, C. Mudivarthi, A. B. Flatau, Stress and magnetic field-dependent Young's modulus in single crystal iron-gallium alloys. J. Magn. Magn. Mater. 322, 2135-2144 (2010)).

Bioelectronics are revolutionizing the future of human life by reshaping fields in communication and personalized healthcare (S. Xu, A. Jayaraman, J. A. Rogers, Skin sensors are the future of health care. Nature 571, 319-321 (2019)). Biomechanical-to-electrical energy conversion is a unique pathway to realize self-powered bioelectronics in the imminent era of Internet of Things (IoT) and fifth-generation (5G) communication (J. Chen, Z. L. Wang, Reviving vibration energy harvesting and self-powered sensing by a triboelectric nanogenerator. Joule 1, 480-521 (2017)). This conversion is round-day available and up to 100 W output easily sustained by an average person (Q. Li, V. Naing, J. M. Donelan, Development of a biomechanical energy harvester. J. Neuroeng. Rehabil. 6, 22 (2009)). On one hand, for sustainable electricity generation, it emerges as a pervasive energy solution to substitute the traditional centralized power grid to meet the energy need of distributed electronics in the era of IoT (Q. Shi, B. Dong, T. He, Z. Sun, J. Zhu, Z. Zhang, C. Lee, Progress in wearable electronics/photonics—Moving toward the era of artificial intelligence and internet of things. InfoMat 2, 1131-1162 (2020)). On the other hand, for the distributed healthcare system, its electrical output can be utilized to retrieve information of various human activities in a self-powered manner. Current biomechanical energy conversion mechanisms, including piezoelectric and triboelectric effects, confront significant limitations such as low current density and high internal impedance, which arise from their capacitive power generation principle via electric dipole alignment (R. D. I. G. Dharmasena, J. H. B. Deane, S. R. P. Silva, Nature of power generation and output optimization criteria for triboelectric nanogenerators. Adv. Energy Mater. 8, 1802190 (2018)). Additionally, without encapsulation, their output performance is vulnerable to the humidity caused by body sweating and ambient fluidic environment, which severely limits their practical deployments in wearable and implantable bioelectronics (W. Yang, W. Gong, C. Hou, Y. Su, Y. Guo, W. Zhang, Y. Li, Q. Zhang, H. Wang, All-fiber tribo-ferroelectric synergistic electronics with high thermal-moisture stability and comfortability. Nat. Commun. 10, 5541 (2019); Z. L. Wang, Triboelectric nanogenerators as new energy technology and self-powered sensors—Principles, problems and perspectives. Faraday Discuss. 176, 447-458 (2014)). Therefore, there exists urgent demand to search for unexploited working principles effective under biomechanical stimulus from several to several hundred kPa (J. Stokes, “Man/System requirements for weightless environments,” (NASA/Marshall Space Flight Center Huntsville, Alabama., 1976) for optimized biomechanical-to-electrical energy conversion, featuring high current output, low internal impedance, and waterproofness.

### A. Summary

Embodiments in accordance with a first aspect include the discovery of a giant magnetoelastic effect in a soft system distinguishable from traditional magnetoelastic effect in rigid metal alloys. By leveraging the magnetic dipole alignments through strong dipole-dipole interaction inside a polymer matrix, giant magnetoelastic effect was created with 4 times larger magnetic field variation under kPa-level biomechanical stimulus compared with conventional magnetoelastic effect. It also requires no external magnetic fields, which greatly broadens its spectrum of applicability. Embodiments further experimentally coupled the giant magnetoelastic effect with magnetic induction, a remarkable current flow would be induced in the external circuit via manipulating the magnetic dipoles, enabling an emerging solution for high-performance biomechanical-to-electrical energy conversion. Relying on this unique coupling effect, a soft MEG was invented as an inductive electrical source with comparable elastic modulus to human skins and tissues (C, Pailler-Mattei, S. Bec, H. Zahouani, In vivo measurements of the elastic mechanical properties of human skin by indentation tests. Med Eng Phys 30, 599-606 (2008); P. G. Agache, C. Monneur, J. L. Leveque, J. De Rigal, Mechanical properties and Young's modulus of human skin in vivo. Arch. Dermatol. 269, 221-232 (1980)). The soft MEG was fully waterproof without encapsulation because magnetic fields can pass through water with negligible intensity loss. It delivers an unprecedented short-circuit current I_{sc} density of 4.27 mA cm⁻² with an internal impedance of ˜30Ω for biomechanical-to-electrical energy conversion. This I_{sc} density is around 10,000 and 10,000,000 times enhancements respectively to that of triboelectric effect (F.-R. Fan, Z.-Q. Tian, Z. Lin Wang, Flexible triboelectric generator. Nano Energy 1, 328-334 (2012)) and piezoelectric effect (R. Yang, Y. Qin, C. Li, G. Zhu, Z. L. Wang, Converting biomechanical energy into electricity by a muscle-movement-driven nanogenerator. Nano Lett. 9, 1201-1205 (2009)) based soft counterparts with internal impedances in the order of several megaohms. The matched internal impedance of soft MEG with commercial electronics realizes a minimum power waste with no requirement for complex management circuitry. Therefore, the soft MEG was able to deliver a power density of 20.17 W m² from human exercises, measure the human pulse wave with perspiration, and performs implantable power generation under ultrasound excitation without the need of encapsulation. It is anticipated that the discovery of giant magnetoelastic effect and the invention of soft MEGs paves a new way for biomechanical energy conversion with a collection of compelling features and opening the doors to a wide range of possibilities.

### A. Details

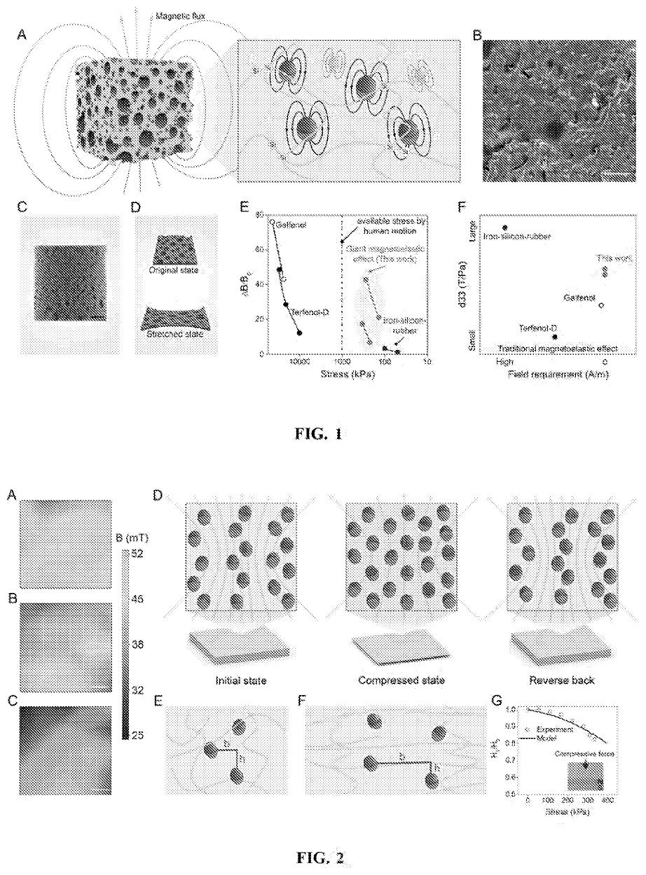

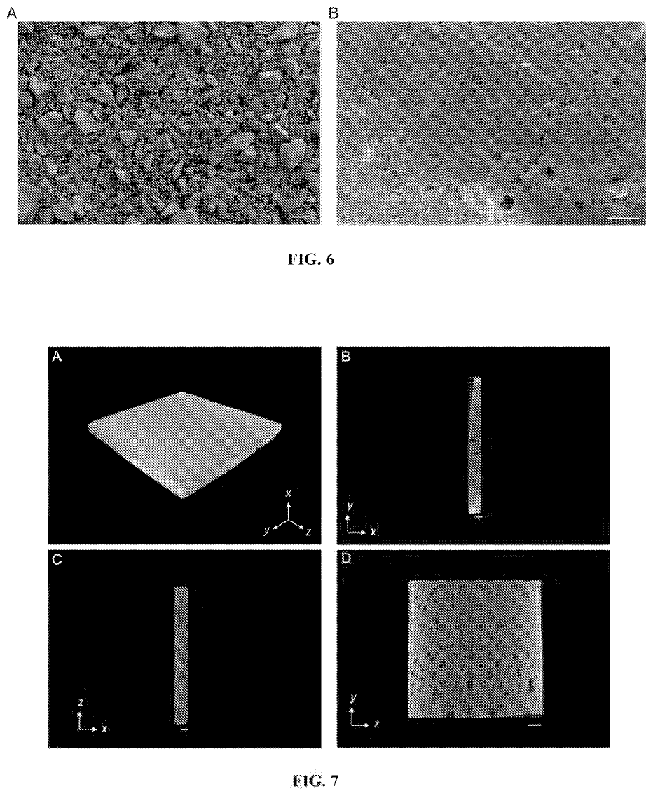

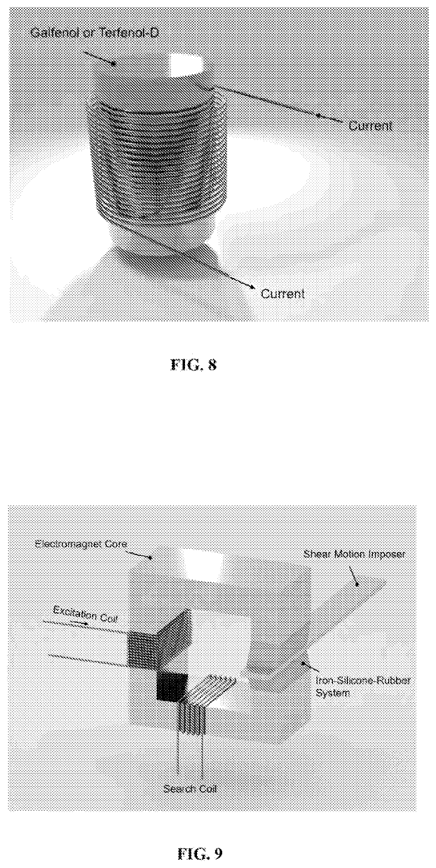

According to certain first aspects, the present embodiments relate to the discovery of a giant magnetoelastic effect in a soft system comprised of magnetizable neodymium-iron-boron (NdFeB) nanomagnets and porous silicone rubber matrix (FIG. 1A). The scanning electron microscopy (SEM) (FIG. 1B, FIG. 6) and micro-computed tomography images (Micro-CT) (FIG. 1C, FIG. 7) reveal the uniform distribution of NdFeB nanomagnets in a porous cellular polymer matrix. Such a porous structure not only reduces the mechanical modulus to render a comfort contact with human skin, but also favors the large mechanical deformation of the soft system under hand stretching, bending, and twisting, improving the biomechanical-to-magnetic energy conversion (FIG. 1D) (J. Kováčik, Correlation between Young's modulus and porosity in porous materials. J. Mater. Sci. Lett. 18, 1007-1010 (1999)).

The soft system demonstrates giant magnetoelastic effect via manipulating the magnetic dipoles. As shown in FIG. 1E, the system alters its surface magnetic flux density up to 20% in the middle and more than 40% in the edge under compressive mechanical stress from 100 to 300 kPa. This value is 4.27 times higher than the magnetoelastic effect in the iron-silicon-rubber system (G, Diguet, G. Sebald, M. Nakano, M. Lallart, J.-Y. Cavaillé, Magnetic particle chains embedded in elastic polymer matrix under pure transverse shear and energy conversion. J. Magn. Magn. Mater. 481, 39-49 (2019)) and comparable to that of Terfenol-D and Galfenol alloy (FIG. 8) which usually requires mechanical stress more than 10 MPa, two orders magnitude higher and well beyond human availability (Z. Deng, Nonlinear modeling and characterization of the Villari effect and model-guided development of magnetostrictive energy harvesters and dampers, The Ohio State University, (2015); J. Liu, C. Jiang, H. Xu, Giant magnetostrictive materials. Sci. China Technol. Sci. 55, 1319-1326 (2012); X. Zhao, D. G. Lord, Application of the Villari effect to electric power harvesting. J. Appl. Phys. 99, 08M703 (2006)). It is worth noting that all the reported magnetoelastic systems, including the iron-silicon-rubber system, need permanent magnets or even bulky electromagnets (FIG. 9) to achieve meaningful d33, while the present soft system does not require external magnetic fields which significantly simplifies the system configuration. In order to further demonstrate the giant magnetoelastic effect in the soft system, compared was the piezomagnetic coefficient d33 to evaluate the performance of different magnetic systems (FIG. 1F and Table A-S1). The present system demonstrates a d33 of 6.73×10⁻⁸ (T/Pa), 5 times and 1.74 times of the values achieved by Terfenol-D (1.36×10⁻⁸) and Galfenol (3.85×10⁻⁸), both of which are among the strongest magnetoelastic metal alloys. It is also worth noting that the d33 of Terfenol-D and Galfenol is achieved in the burst region under MPa-level stress while the highest d33 of the present system is obtained under kPa-level biomechanical stimulus within human availability

TABLE A-S1

| | | External | Applied |
|---|---|---|---|
| | Piezomagnetic | magnetic | pressure |
| Systems | coefficient (T/pa) | field (kA/m) | range (kPa) |
| Iron-Silicon- | 10 × 10⁻⁷ | 160000 | 0-100 (shear) |
| Rubber | | | |
| Terfenol-D | 1.36 × 10⁻⁸ | 79770 | 11000-41000 |
| Galfenol | 3.85 × 10⁻⁸ | 5500 | 10000-50000 |
| This work | 6.73 × 10⁻⁸ | 0 | 0-350 |
| | (middle) | | |
| | 6.29 × 10⁻⁸ | | |
| | (edge) | | |

FIGS. 2A-C show the magnetic flux density mapping on the surface of the soft system under different mechanical stresses of 0, 139 and 278 kPa, respectively. The magnetic flux density decreased significantly both in the middle and the edge of the surface. It is worth noting that both the absolute magnetic flux density and flux density variation are higher in the edge, owing to the cancellation of the magnetic field at the center of the surface. The observed giant magnetoelastic effect results from different mechanisms compared to the traditional magnetoelastic effect in Terfenol-D and Galfenol alloys. The latter arises from the realignment of magnet domains and stress-induced magnetic anisotropy under external magnetic fields (M. Sheikh Amiri, M. Thielen, M. Rabung, M. Marx, K. Szielasko, C. Boller, On the role of crystal and stress anisotropy in magnetic Barkhausen noise. J. Magn. Magn. Mater 372, 16-22 (2014)) while the giant magnetoelastic effect in the present soft system is attributed to the change of nanomagnet chain structure under mechanical deformation. As illustrated in FIG. 2D, the NdFeB nanomagnets are considered as single magnetic dipole and aligned in a wavy chain structure in the initial state after magnetization. In the compressed state, the nanomagnet chain structure varies and alternates the dipole-dipole interaction inside the chain associated with decrease of magnetic flux density. Once the stress is released, the recovery of the nanomagnet wavy chain structure reverses the magnetic flux density back. FIGS. 2E and 2F depict the variation of magnetic domain inside the wavy chain with changed dipole distance and distribution. FIG. 2G plots the experimental results of decreasing magnetic field flux with increasing compressive stress. Based on the wavy chain model, the relationship between the magnetic field H and principal stretch λ could be expressed as (supplementary text),

$$H = N \frac{\text{?}}{\text{?}} \frac{4}{3} \text{?} + \frac{\text{?}}{\text{?}} \frac{\text{?}}{\text{?}} ( \frac{1}{2} f ( \frac{\text{?}}{\text{?}} ) - 0.1503 ) \qquad ( 1 )$$

$$\text{?} \text{indicates text missing or illegible when filed}$$

where N is the number of the nanomagnets, r is the particle radius (12.5 μm on average), Br is the remnant magnetic flux density of the nanomagnets, μ0 is the permeability of vacuum space, λ is the stretch in the compress direction, b and h are horizontal and vertical distances between the neighboring particles, 0.5f(x)−0.1503 is the dipole alignment factor, which describes the contribution of all other dipoles to the magnetic interaction energy of a single dipole in the wavy chain. Under the assumption of incompressible Neo-Hookean solid, the magnetic field H is further linked to applied nominal stress s in the soft system through,

$$\text{?} = \text{?} ( λ - \frac{1}{\text{?}} ) \qquad ( 2 )$$

$$\text{?} \text{indicates text missing or illegible when filed}$$

where G is the shear modulus. With the measured b and h value of 43.5 μm, the wavy chain model accurately captures the decrease of magnetic field ratio H1/H0 with applied compressive stress s and fits well with the experimental results in FIG. 2G.

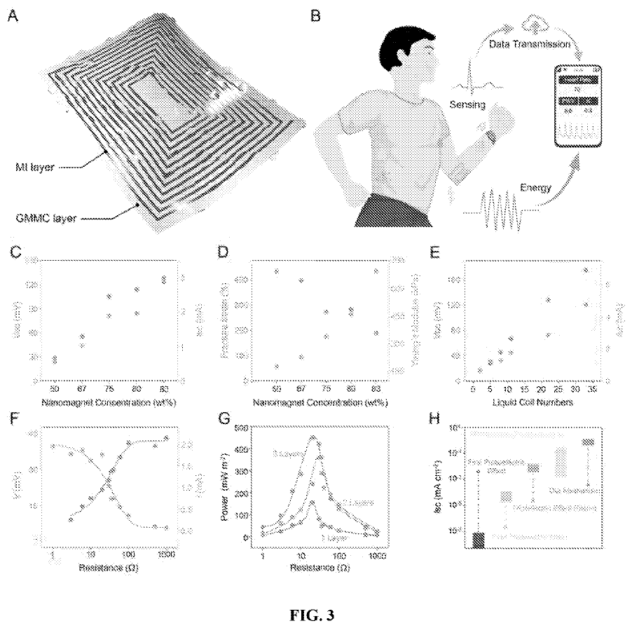

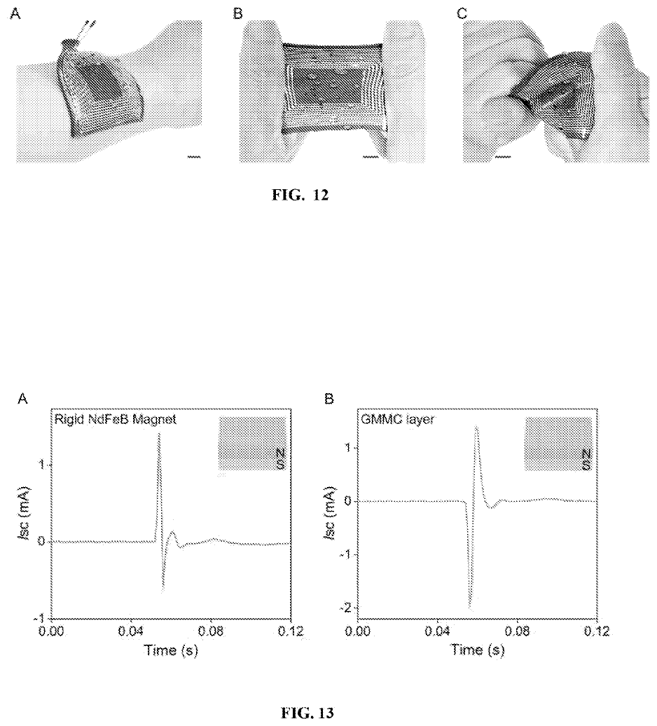

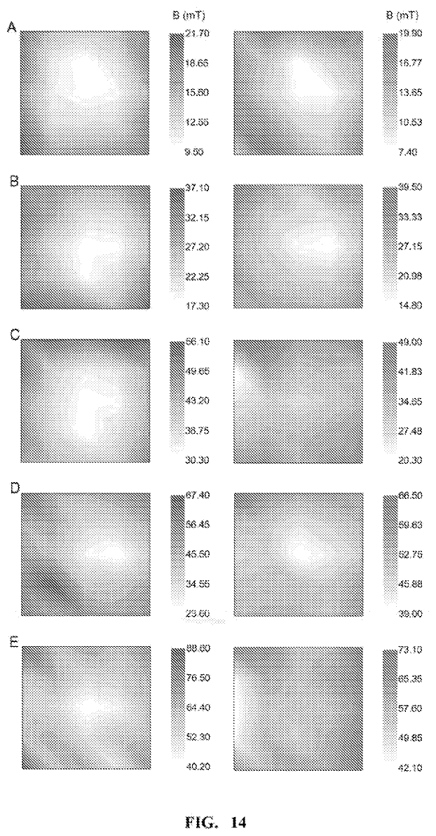

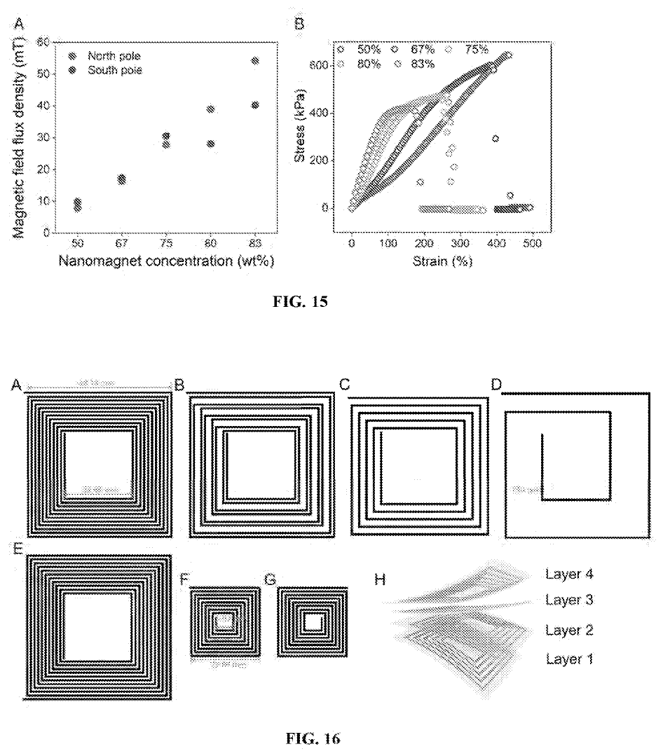

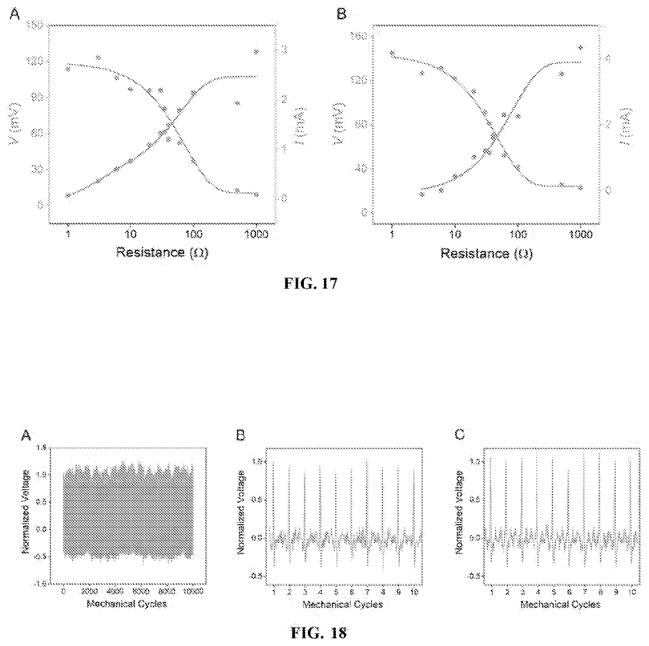

The giant magnetoelastic effect is able to convert the tiny pressure to: significant localized magnetic field variation via manipulating the magnetic dipole in a soft matrix, which could be further utilized to generate electricity if magnetic induction could be introduced to realize magnetic to electrical conversion. Thus, a soft MEG was developed with a giant magnetomechanical coupling (GMMC) layer and a patterned liquid metal receiver as the magnetic induction (MI) layer (FIG. 3A). Its working mechanism holds a two-step conversion with the giant magnetoelastic effect for biomechanical-to-magnetic conversion and the magnetic induction for magnetic-to-electrical conversion. Since the magnetic field variation can pass through the water without significant intensity decrease, the soft MEG is intrinsically waterproof and could operate stably against heavy perspiration (FIG. 3B). With an all-in-one design, the soft MEG can be bent, twisted, stretched to arbitrary shapes, and attached to human skin conformably (FIG. 12). According to Faraday's law, the electric output of the soft MEG is proportional to both the magnetic field variation in the GMMC layer and the number of the liquid metal cycles in the MI layer, which could be optimized for an enhanced biomechanical-to-electrical energy conversion. First, for the GMMC layer, nanomagnet concentration is a critical parameter, Higher concentration of nanomagnets will lead to larger magnetic field variation and better electrical output (FIG. 3C and FIG. 14). However, the softness of the GMMC layer follows a reverse trend with the increase of the nanomagnet concentrations (FIG. 3D and FIG. 15). As a result, 83 wt % is the optimized value which assures both the large magnetic field variation and mechanical softness. Furthermore, regarding the MI layer, in order to increase the number of liquid metal cycles in a limited surface area, multilayer structure was adapted as displayed in FIG. 16. MI layers of 2, 5, 8 and 11 liquid metal cycles in the 1-layer structure, 22 liquid metal cycles in 2-layer structure as well as 33 liquid metal cycles in the 3-layer structure were fabricated using the same patterning technique and characterized systematically in FIG. 3E. It is clear that both open-circuit voltage (Voc) and Isc show a linear relationship with the number of liquid metal cycles in the MI layer. With the 3-layer structured MI layer, Voc and Isc of soft MEG are measured to be 175 mV and 4.77 mA, respectively, corresponding to a peak power of 450 mW m⁻²¬at the matched load resistance of 20 (2 (FIGS. 3F and 3G, FIG. 17). The combining effect of giant magnetoelastic effect and magnetic induction in the present soft MEG distinguishes itself in the community of biomechanical energy conversion compared with other counterparts based on triboelectric effect and piezoelectric effect in the literature (L. Gu, J. Liu, N. Cui, Q. Xu, T. Du, L. Zhang, Z. Wang, C. Long, Y. Qin, Enhancing the current density of a piezoelectric nanogenerator using a three-dimensional intercalation electrode. Nat. Commun. 11, 1030 (2020); B. Dudem, A. R. Mule, H. R. Patnam, J. S. Yu, Wearable and durable triboelectric nanogenerators via polyaniline coated cotton textiles as a movement sensor and self-powered system. Nano Energy 55, 305-315 (2019)). Via electric dipole alignment, triboelectric and piezoelectric effect-based electricity generation relies on the capacitive conduction, leading to a low current output via electric dipole polarization (Z. L. Wang, On Maxwell's displacement current for energy and sensors: the origin of nanogenerators. Mater, Today 20, 74-82 (2017)). On the contrary, the alignment alternation of nanomagnetic dipoles both on and inside the surface of the GMMC layer can be harvested through MI layers remotely, boosting the current output of the soft MEG to a much higher level for biomechanical energy harvesting. It is believed that the measured current density of more than 1 mA cm⁻² is about 10,000 and 10,000,000 times higher than the first biomechanical energy harvesters based on triboelectric effect and piezoelectric effect, respectively (FIG. 3H). More detailed comparisons can be found in Table A-S2. The high current density, low internal impedance and waterproof capability of the present soft MEG render it an emerging approach for biomechanical energy conversion. It can also charge small electronics much faster without reliance on the complex management circuits owing to the matched internal impedance. In addition, the soft MEG is intrinsically stable against human sweating and other environmental contaminants, which is a significant and pendent challenge for the existing working mechanisms in the community of biomechanical energy conversion.

TABLE A-S2

| | | | | Internal | | Peak power | | |
|---|---|---|---|---|---|---|---|---|
| Mechanism | V_{OC} | I_{SC} | | impedance | | density | | Ref |
| Giant magnetoelastic | 1.38 V | 4.27 | mA cm⁻² | ~30 | Ω | 20.17 | W m⁻² | This work |
| effect, magnetic induction | | | | | | | | |
| First Triboelectric effect | 3.3 V | 0.11 | μA cm⁻² | — | | 10.4 | mW cm⁻³ | 17 |
| First Piezoelectric effect | 0.15 V | ~0.1 | nA cm⁻² | — | | — | | 18 |
| Triboelectric effect record | 350 V | 11.25 | μA cm⁻² | 10⁸ | Ω | 11 | W m⁻² | 25 |

| | | | | | | |
|---|---|---|---|---|---|---|
| Piezoelectric effect record | 28 V | 0.29 | mA cm⁻² | — | — | 26 |

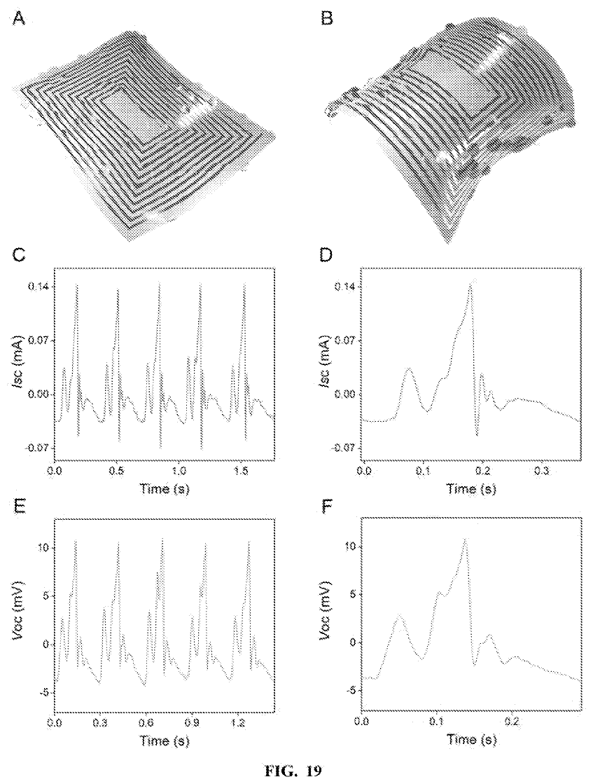

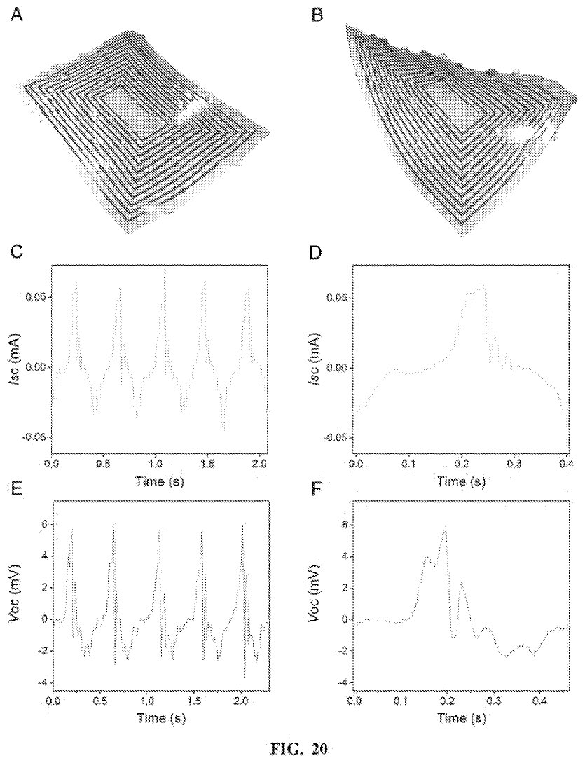

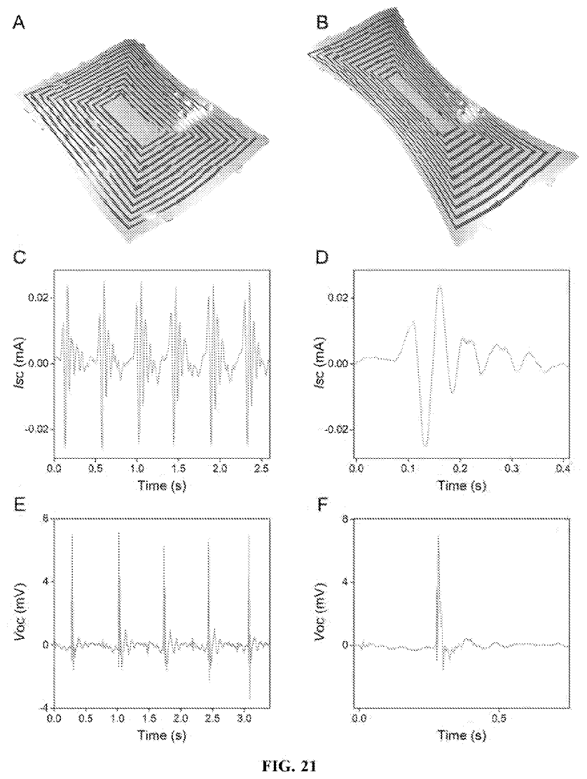

The long-term durability of soft MEG is examined with repeated mechanical compress-relax cycles at 20 Hz and the results show that it remains constant electric output after 10,000 loading-unloading cycles (FIG. 18). Besides the compressive stress, the soft MEG can also generate voltage up to 10 mV and current up to 0.14 mA when bent, twisted, or stretched via hand, as demonstrated in FIGS. 19-21. Although these peak electrical outputs are smaller than that under the compressive deformation, the duty cycle under these mechanical deformations is on the order of 0.1 s, much longer than that under compressive deformation, indicating a similar or even higher charge generation.

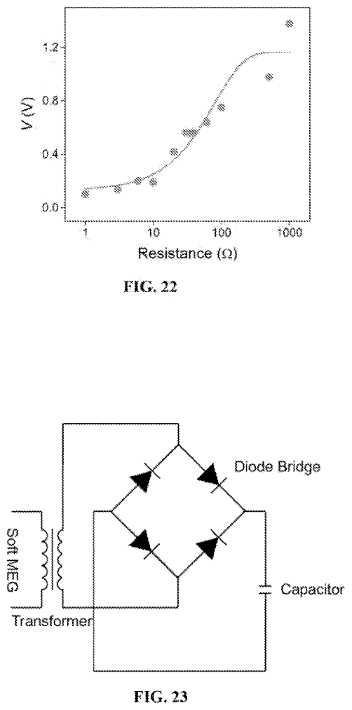

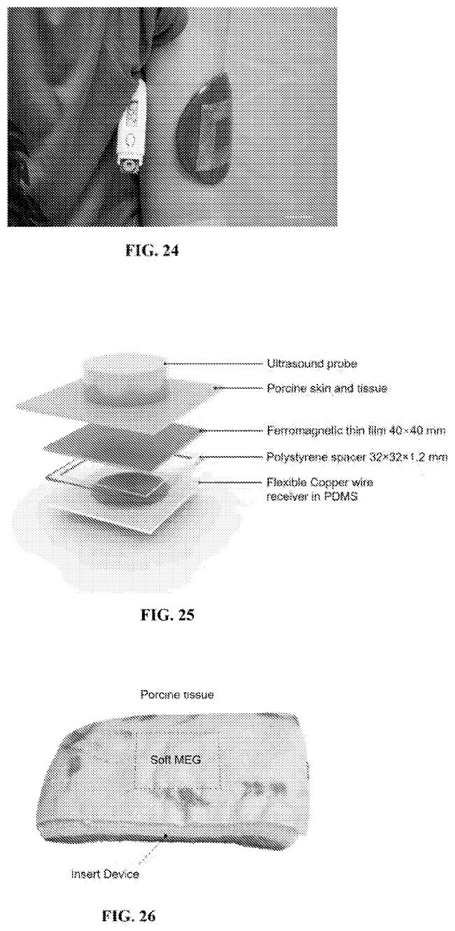

The high-performance biomechanical-to-electrical energy conversion enabled by the soft MEG is capable of driving wearable bioelectronics, including a thermometer, a sweat sensor, and an electrocardiogram for personalized healthcare (FIG. 4A). A soft MEG with an MI layer of 300 soft copper cycles is able to deliver an Isc of 4.27 mA cm⁻², Voc of 1.38 V and a peak power of 20.17 W m⁻² with an internal impedance of 30 (2 (FIG. 4B and FIG. 22). FIGS. 4C and 4D present the Voc and I_{sc} of the soft MEG under continuous hand tapping. The electrical output was further boosted and regulated using a toroidal transformer and a Schottky diode bridge (FIG. 23). Benefiting from the high output current of the soft MEG, with gentle hand tapping, capacitors of 22 μF, 47 μF and 100 μF were successfully charged to 3 V, 2 V and 1.1 V within 33 seconds, respectively (FIG. 4D). A soft MEG with MI layer of 22 liquid metal cycles was also demonstrated to sustainably drive a commercial thermometer for continuous body temperature monitoring (FIG. 24).

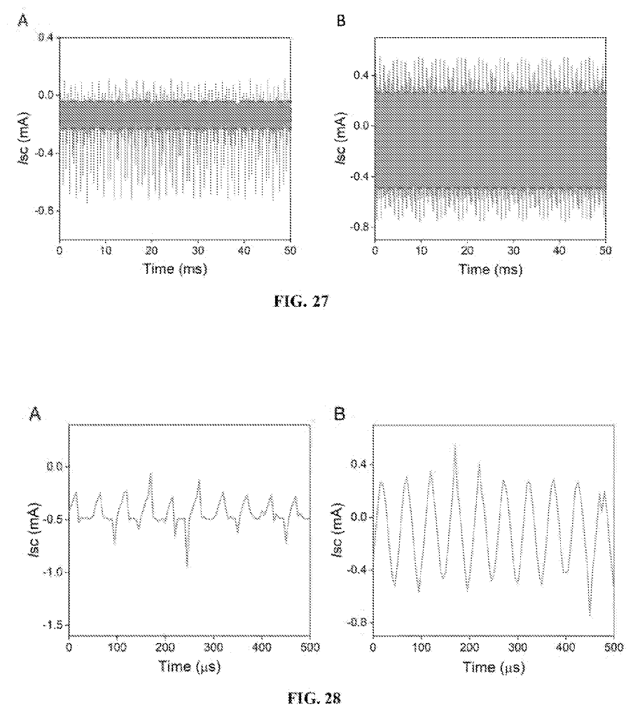

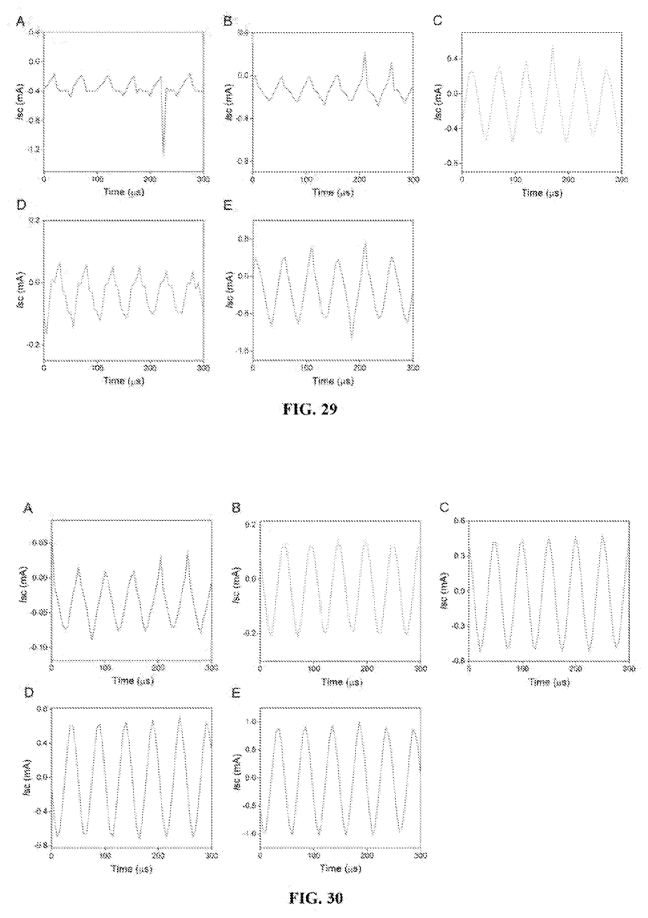

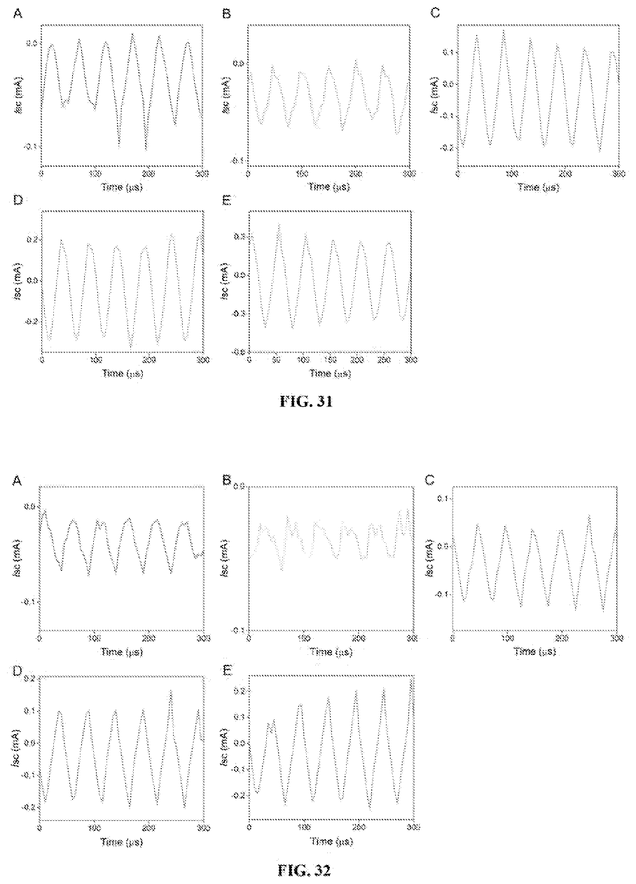

Apart from wearable power generation, sustainable energy supply for implantable electronic devices remains highly desired but a challenging technique. External recharging technology including inductive coupling have low efficiency and safety concerns of tissue damage. Acoustic waves and ultrasound are safe at low power and can transfer energy in vivo regardless of environmental conductivity and transparency. Therefore, they have been used in disease sensing, diagnosing, and monitoring as well as energy transmitting for medical implants in vivo with triboelectric and piezoelectric energy harvesters (R. Hinchet, H.-J. Yoon, H. Ryu, M.-K. Kim, E.-K. Choi, D.-S. Kim, S.-W. Kim, Transcutaneous ultrasound energy harvesting using capacitive triboelectric technology. Science 365, 491 (2019)). However, a significant challenge of current implantable ultrasound energy harvesters is the requirement of encapsulation layers to enhance the biocompatibility and prevent the adverse effect of body fluids. The encapsulation layer absorbs a large amount of ultrasound and therefore reduces the energy conversion efficiency (Id.). In contrast, soft MEG possesses outstanding electric output under water without the need of encapsulation because of the negligible influence of water on the magnetic field. Demonstrated is the possibility of using the soft MEG as an implantable power source to harvest ultrasound excitation without encapsulation under porcine tissue (FIG. 4F). As illustrated in FIG. 4G, the soft MEG with the MI layer of 100 soft copper cycles implanted 5-15 mm underneath the skin (FIGS. 25 and 26). Control experiments were first performed to identify that the electric output was mainly from mechanical motion of the GMMC layer instead of the wireless transmission (FIG. 27 and supplementary text below). Then an optimal device structure of the soft MEG (FIGS. 28 and 29) is determined at the implantation depth of 5 mm under 20 kHz ultrasound excitation. Ex vivo characterized was the optimized soft MEG at different implantation depths with different ultrasound power densities, as the results shown in FIG. 4H and FIGS. 29-31. Performance of the optimized soft MEG increases with increasing ultrasound power and decreasing implantation depth which can be ascribed to attenuation of the ultrasound inside the layer-structured porcine tissue. The soft MEG exhibits the highest output current of approximately 0.94 mA at 5 mm under ultrasound excitation with 100% power output. This value is approximately 5 times larger than a TENG with the similar testing conditions (Id.). FIG. 4I demonstrates the waveform of the soft MEG, which is sinusoidal and of the same frequency. The dependence of the output current and power on the load resistances is demonstrated in FIG. 4J. Peak power of 30.69 μW is reached at 100Ω, which is comparable to many implantable bioelectronics such as pacemakers and neurostimulators indicating the practicability of the soft MEG as an implantable power source (Id.).

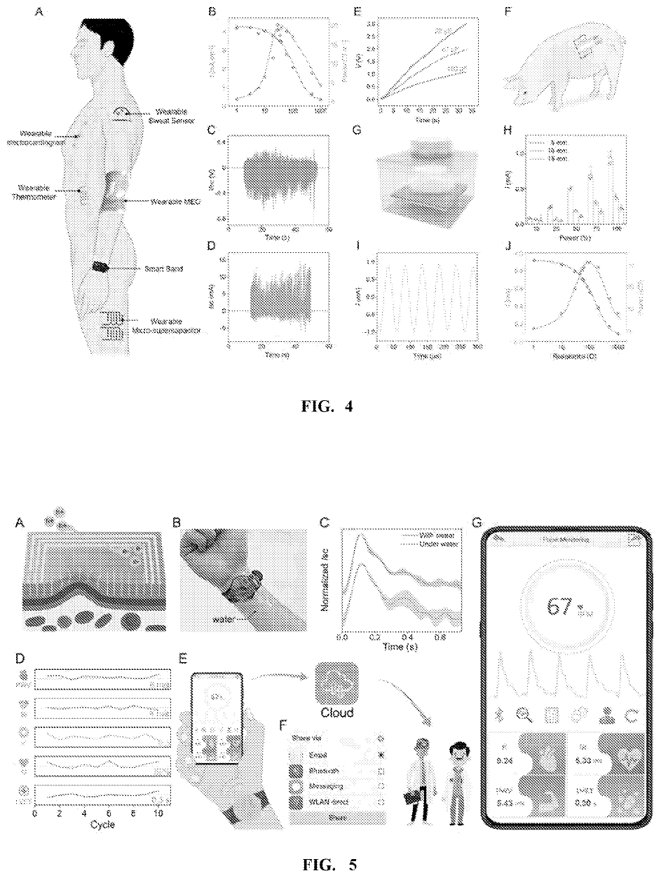

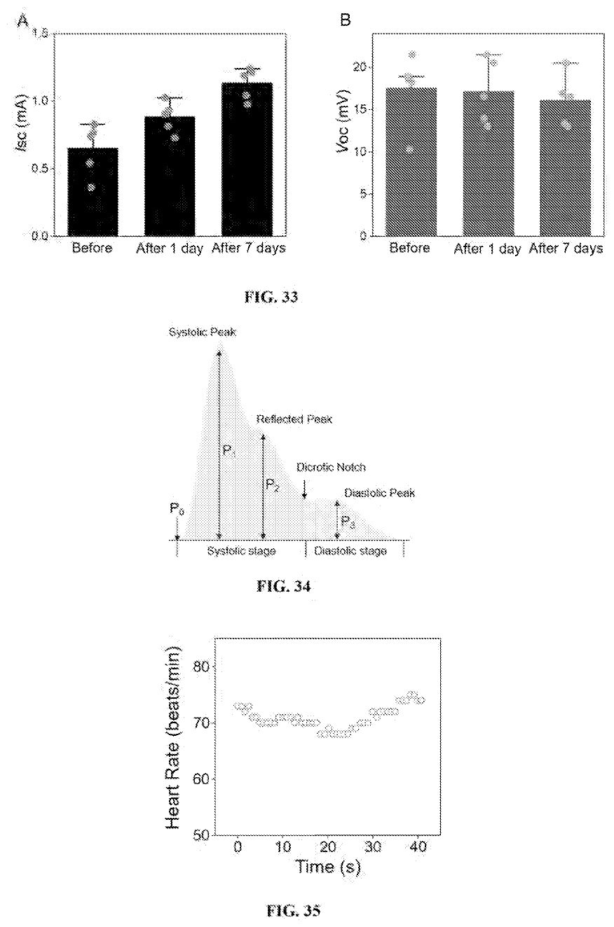

The sweat-resistant feature is important for continuously monitoring human physiological signals since sweating is unavoidable and can amount to as much as 10 liters every day (B. M. Marriott, Nutritional Needs in hot environments: Applications for military personnel in field operations. (National Academies Press, 1993)). Current working mechanisms require encapsulation layers to be sweatproof, which significantly reduce the sensitivity (Q. Zheng, H. Zhang, B. Shi, X. Xue, Z. Liu, Y. Jin, Y. Ma, Y. Zou, X. Wang, Z. An, W. Tang, W. Zhang, F. Yang, Y, Liu, X. Lang, Z. Xu, Z. Li, Z. L. Wang, In Vivo Self-Powered Wireless Cardiac Monitoring via Implantable Triboelectric Nanogenerator. ACS Nano 10, 6510-6518 (2016)) Beyond the wearable/implantable energy harvesting, embodiments also demonstrate the feasibility of soft MEG as a self-powered and sweatproof biosensor to monitor human arterial pulse underwater or in a sweaty condition. The weak human pulse vibration leads to the deformation of the conformally attached soft MEG and causes a magnetic field distortion through the MI layer, which induces an electromotive force and generates the electric signal (FIG. 5A). As shown in FIG. 5B, the soft MEG could be conveniently worn against the wrist for human pulse monitoring in a wet state. Based on the giant magnetoelastic effect, the output electric current and voltage of MEG self-powered pulse sensor maintain constant even after submerged in artificial perspiration for 7 days (FIG. 33). In order to demonstrate the waterproof feature of the soft MEG based pulse sensor toward practical applications, tested was the device both under water and with human sweat (FIG. 5C). The obtained signals are similar in both situations and contain fine structures such as systolic and diastolic peaks in the waveform benefiting from the high sensitivity. As a result, key parameters including stiffness index (SI), pulse wave velocity (PWV), K value, augmentation index (AI) and left ventricular ejection time (LVET) can be extracted accurately to evaluate the cardiovascular health status (FIG. 5D and FIGS. 34-36, supplementary text below). All the cardiovascular parameters withdrawn from the wearable device denote a healthy heart state of the human subject and validate the availability of soft MEG as a self-powered human pulse monitor capable of detecting imperceptible pressure changes.

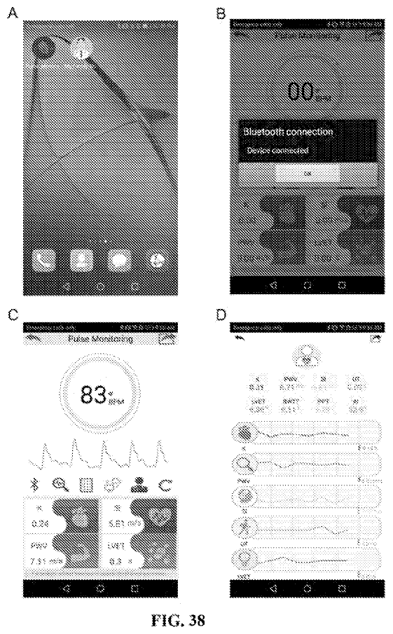

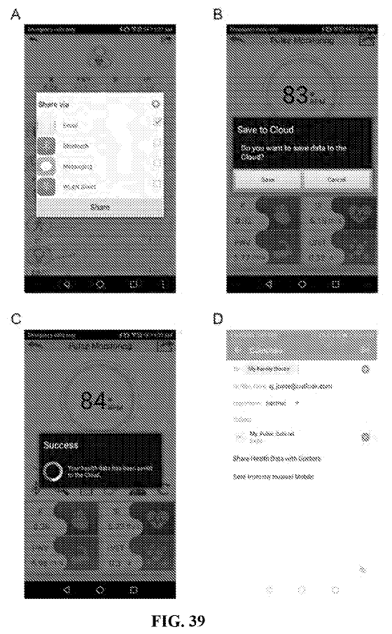

Further developed was a highly-integrated cardiovascular health monitoring system including the self-powered pulse wave sensor, an analog front-end for signal amplification and filtration, a micro-controller for data processing, and a customized health monitoring cellphone application (APP) for data display, storage and sharing (FIG. 37). As illustrated in FIG. 5E, the pulse signal measured by the soft MEG is first amplified and filtered by an analog circuit to ensure enough details of pulse waveforms. The analog signal is then converted to digital signal through a micro-controller unit (STM32) and wirelessly transmitted to the cellphone APP through an on-board Bluetooth module. The customized cellphone APP with a built-in algorithm can display the cardiovascular system measurement in the front-ends, including real-time pulse waveforms as well as cardiovascular parameters such as PWV, K and SI to track the cardiovascular health status of a human subject (FIG. 5G and FIG. 38). All the measured health data can be one-click forwarded to physicians through email, cloud service or message as displayed in FIG. 5F and FIG. 39. The present integrated cardiovascular health monitoring system can continuously and stably provide real-time, patient-generated health data, and wirelessly display it in the cellphone APP, in the heavily sweaty situation and even with artificial perspiration spraying (Movie S2). Without the need of encapsulation induced bulky structure and less sensitivity, the waterproof health monitoring system represents a unique and compelling bioelectronic that practically enables the change of the current reactive and disease-centric healthcare system to a personalized model with a focus on disease prevention and health promotion.

### A. Conclusions

The present Applicant discovered the giant magnetoelastic effect in a soft system and proposed an analytical model for its mechanism interpretation. Through leveraging magnetic dipole alignments in a soft matrix, giant magnetoelastic effect yields a 4 times magnetomechanical coupling efficiency larger than conventional magnetoelastic effect with additional advantages, such as mechanical softness and no requirement of external magnetic field. Towards practical applications, a soft MEG combining giant magnetoelastic effect and magnetic induction was invented and introduced to the biomechanical energy conversion community, which explicitly addresses the long lasting and fundamental challenges in the community, including low current, high internal impedance, and vulnerability to ambient humidity. The waterproof soft MEG showed an ultralow internal impedance of ˜30Ω, delivers a Isc up to 4.27 mA cm⁻², which is 10,000 times outperforming other soft counterparts for biomechanical energy harvesting. It was therefore demonstrated as a high-performance wearable/implantable power source without encapsulation, as well as a waterproof and self-powered biosensor for imperceptible human pulse monitoring. With a collection of compelling features, the present soft MEG represents a first step towards a pervasive energy solution that fuses high-current power supply and self-powered waterproof biosensing for the self-sustained operation of trillions sensor nodes of versatile modalities in the era of IoT. It thus can be regarded as the milestone in the context of biomechanical-to-electrical energy conversion community for human-centered energy, sensing and therapeutic applications. It also establishes the foundation and would bring new blood to many fields, including energy harvesting, human-machine interface, medical electronics, and soft robotics.

### A. Example Materials and Methods

### Fabrication of Giant Magnetomechanical Coupling (GMMC) Layer.

GMMC layer is prepared by thoroughly mixing uncured silicone rubber matrix with non-magnetized NdFeB nanomagnets and then cured in a heat oven with introduced air bubbles in micrometer scale. Specifically, Ecoflex 00-30-part A, part B and ferromagnetic powder with an average size of 25 μm (MQFP-B-20076-088) were blended thoroughly using a stirring rod. Vacuum degassing is not performed thereafter in order to introduce air bubbles for porous structure. The mixture was then cured at 60° C. in an oven (ThermoFisher) for 3 hours. The non-magnetized elastomer was magnetized by impulse a magnetic field (approximately 2.655 T) using an impulse magnetizer (IM-10-30, ASC Scientific) to import stable remnant magnetization. The weight ratio of Ecoflex 00-30-part A and part B is kept at 1:1 for all GMMC layers. The weight percent (wt %) of ferromagnetic powder in the silicone elastomer varies from 50% to 83% to fabricate the GMMC layer with different magnetic and mechanical properties.

### Characterization of the GMMC Layer.

Morphology and the internal structure of the GMMC layer was imaged by SEM (ZEISS Supra 40VP) and in-house Micro-CT (crumpCAT). The magnetic flux density mapping on the 2×2 cm² surface was created by measuring magnetic flux density of 25 evenly distributed spots with a digital gauss meter.

Mechanical performances of the GMMC layer (width 5 mm, gauge length 4.5 mm) were determined using tensile testing by a dynamic mechanical analyzer (DMA, RSA III). The nominal stress-strain curves of the GMMC layer were plotted in FIG. 15. The Young's modulus and shear modulus were obtained by fitting the experimental curves with a Neo-Hookean model. The magnetized GMMC layer (83 wt %) demonstrate a shear modulus of 230.74 kPa, Young's modulus of 692.23 kPa and an ultimate strain of 189.28%, respectively.

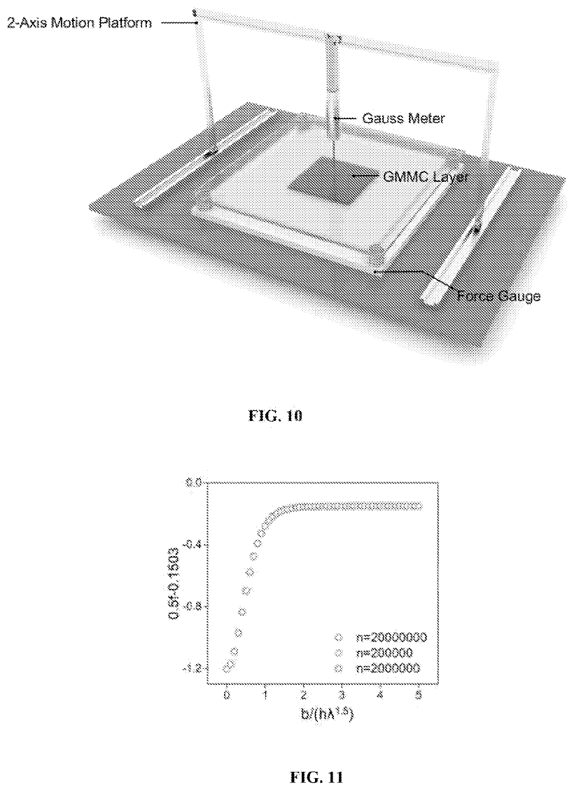

The magnetic flux density mappings on the 2× 2 cm² GMMC layer surface under applied pressure (0, 139, and 278 kPa) was created using an experimental setup in FIG. 10. The magnetic flux density in the middle of the GMMC layer surface was measured under applied pressure from 0 to 333.62 kPa consecutively to verify the proposed the wavy chain model.

### Fabrication of the Magnetic Induction (MI) Layer.

Ga (99.99%) and In (99.99%) ingots were purchased from RotoMetals. EGaIn (74.5% Ga and 25.5% wt % In) was prepared by heating in a muffle furnace (ThermoFisher) at 200° C. for 2 hours. Then, 10 wt % Ni microparticle (99.5%, 5 μm, US Research Nanomaterials) was added and mixed thoroughly using a VWR mini Vortexer to achieve preferred rheological property for improved processability before any usage. A laser cutting machine (ULTRA R5000, Universal Laser System) was used to cut a PET film as a square helix mask (outer length 48.18 mm, inner length 22.86 mm, linewidth 730 μm). Liquid metal was then patterned onto a thin polydimethylsiloxane substrate (PDMS, Sylgard 184, 40:1) using the PET film mask. Different MI layers (2, 5, 8, and 11 liquid metal cycles) were fabricated using the same patterning technique with the square helix mask of the same dimension and linewidth (FIG. 16).

For the multilayered MI layer, the mask of the second layer (e.g. counterclockwise from outside to inside) is cut in a direction opposite to the masks of first and third layer (e.g., clockwise from outside to inside) to ensure the overall clockwise direction. After patterning each layer, a scotch tape was used to protect the vertical interconnect access and expose it when patterning the next layer. A thin layer of PDMS (40:1) was used to separate and encapsulate each layer without constraining its mechanical stretchability.

### Fabrication of Soft Magnetoelastic Generators (MEGs)

For optimization, a thin PDMS layer (40:1) was used as the substrate, then MI layers of different cycles and different layers were patterned on the substrate. A 2×2 cm²¬GMMC layer of different nanomagnet concentrations was then attached on the surface of the MI layers to form a soft MEG. For bending, twisting, and stretching test, a thin layer of PDMS (40:1) was coated on the GMMC layer with 75 wt % nanomagnet concentration as a substrate. Then a MI layer (2 layers, 11 liquid metal cycles each layer, outer length 48.18 mm, inner length 22.86 mm, linewidth 730 μm) was patterned on the PDMS/GMMC substrate. For self-powered cardiovascular management system, a thin layer of PDMS (40:1) was coated on the GMMC layer (83 wt %) as a substrate. Then a MI layer (2 layers, 8 liquid metal cycles each layer, outer length 22.86 mm, inner length 5.82 mm, linewidth 730 μm) was pattern on the PDMS/GMMC substrate.

### Characterization of Soft MEGs.

A control experiment was first performed to verify the dominance of giant magnetoelastic effect in biomechanical-to-electrical energy conversion in MEG which distinguishes itself from the traditional electromagnetic generator (EMG, FIG. 23). Voltage signals of soft MEGs were measured by a Keithley system electrometer (Keithley 6514). Current signals were measured by a Stanford low-noise current pre-amplifier (Model SR570). Stability of the soft MEG (with MI layer of 22 liquid metal cycles in a 2-layer structure) was tested by a calibration electrodynamic transducer (Labworks Inc. ET-126HF) at 20 Hz. For wearable power generation applications, a Schottky diode bridge rectifier (MBSK16SE) was used for AC/DC conversion. A toroidal transformer was used to elevate the output voltage. For implantable power generation applications, an ultrasonic homogenizer (FS-550T) with a 13 mm probe was used. Its output power was calibrated to be from 0 to 330 W. The ultrasound probe was grounded to minimize the noise. In the characterization of implantable MEGs, ferromagnetic powder with an average size of 5 μm (MQFP-B-20076-089) was used to fabricate the GMMC layer (83 wt %) because it demonstrates larger output signal in control experiments (FIG. 29).

Artificial perspiration was used to test the sweatproof and waterproof abilities of the soft MEG. To prepare the artificial perspiration, 0.455 g KCl (Sigma Aldrich), 1.55 g NaCl (Sigma Aldrich), 0.0583 g Na2SO4 (Sigma Aldrich), 0.252 g NaHCO3 (Sigma Aldrich), 0.182 g 1 M NH3·H2O (Sigma Aldrich), 0.601 g Urea (Alfa Aesar), 0.0092 g uric acid (Alfa Aesar) and 1.26 g 1 M lactic acid solution (Alfa Aesar) were added in 1 L deionized (DI) water. The GMMC layer was submerged in the artificial perspiration for 24 h and 168 h and then tested its electrical output, respectively

### Circuit and Mobile APP Design.

Hardware signal processing circuit consists of four components: an analog pulse signal acquisition, an amplifier, a low-pass filter, and a micro-controller with a Bluetooth transmission module to a customized mobile phone terminal application. In cardiovascular management process, analog pulse signal was collected from the present soft MEG and then amplified by an amplifier (MCP6001) and filtered by a low-pass filter (OP07) to remove both interference signals and environmental noise. The amplification and filtration processes ensure precise expression of the final analog output signal with sufficient details for subsequent processing by an analog-to-digital converter (ADC). Thereafter, a micro-controller unit (STM32) is used to collect and convert the analog pulse signal to digital pulse signal. Finally, a Bluetooth module (HC-05) is applied to transmit the digital signal to the mobile APP via wireless communication.

A customized Android application (named Cardiovascular Health Management_MEG) was developed via MIT A12 Companion for personalized healthcare with friendly user experience. The APP is designed to continuously acquire pulse signals and in situ analyze health status via heart rate (HR) and other key cardiovascular parameters, such as pulse wave velocity (PWV) and stiffness index (SI), upstroke time (UT) and so on.

### A. Supplementary Text

### 1. Theoretical Models to Explain the Giant Magnetoelastic Effect.

It has been shown that under compressive stress, the GMMC layer demonstrated a decreased magnetic flux density on its surface which can be used for mechanical-to-magnetic energy conversion. While many theories have been established to evaluate the magnetostrictive behavior of magnetorheological elastomers. There are few theoretical studies focusing on the inverse magnetostrictive effect of the GMMC layer in the present study. First considered was rotation model to explain the observed magnetoelastic effect.

Embodiments consider each NdFeB particle as a single magnetic dipole and no dipole-dipole interaction to simplify the calculation. Embodiments further assume that after impulse magnetization, the magnetization direction of the NdFeB magnetic dipole follows a normal distribution with its average orientation perpendicular to the surface of the elastomer (same as the magnetization direction). Additionally, embodiments consider that the contribution of a small volume fraction of the GMMC layer to the magnetic field can be approximated by NM, where N is density of magnetic dipole in the small volume and M is average magnetization of a magnetic dipole in this small volume and can be obtained by the following equation

$$M = {\int }_{\text{?}}^{\text{?}} {M}_{\text{?}} \times cos α \times \frac{1}{\text{?}} \times {\text{?}}^{- \frac{\text{?}}{\text{?}} {( \frac{\text{?}}{\text{?}} )}^{\text{?}}} d α \qquad ( 1 )$$

$$\text{?} \text{indicates text missing or illegible when filed}$$

where Ms and a is the magnetization and magnetization angle of a single magnetic dipole. σ is the standard deviation characterizing magnetic dipole distribution. It should be noted that the direction of M is parallel to the impulse magnetization since the magnetization in other direction is canceled by each other. Based on these assumptions, one can calculate the perpendicular magnetic flux density on the middle of the GMMC layer surface (in a 2-dimensional approximation) using the equation below

$${B}_{0} = \frac{\text{?}}{4 π} {\int }_{0}^{\text{?}} {\int }_{0}^{\text{?}} \frac{2 \text{?}}{\text{?}} ( 2 {cos}^{2} ( arctan ( \frac{l}{h} ) ) - 1 ) dldh \qquad ( 2 )$$

$$\text{?} \text{indicates text missing or illegible when filed}$$

where h0 and l0 is the height and half length of the GMMC layer, respectively. μ0 is the vacuum permeability. This equation indicates that under the assumption of geometry invariation, the magnetic field density is decided by M. The decrease of M under compressive stress leads to the decrease of magnetic flux density on the surface of the GMMC layer.

For a single elliptical particle, its rotation in a soft matrix can be approximated by the following equation (3)

$$θ = \frac{\text{?} ( 1 + κ )}{\text{?} ( \text{?} + \text{?} )} sin 2 α \qquad ( 3 )$$

$$\text{?} \text{indicates text missing or illegible when filed}$$

where s is compressive stress, C_ellipse=(a−b)/(a+b), a and b are the major and minor semi-axes of the elliptical particle, respectively. G is the shear modulus of the GMMC layer, κ is Kolosov's constant and equal to 5/3. α is the angle of magnetization Ms of a single dipole with respect to the perpendicular direction.

Using the above equation, one can calculate the average magnetization of a si dipole under applied compressive pressure s as below,

$${M}_{1} - {\int }_{\text{?}}^{\text{?}} {M}_{\text{?}} \times cos ( α + \frac{\text{?} ( 1 + κ )}{\text{?} ( \text{?} + κ )} sin 2 α ) \times \frac{1}{\text{?} \sqrt{2 π}} \times {\text{?}}^{- \text{?} {( \text{?} )}^{\text{?}}} d α \qquad ( 4 )$$

$$\text{?} \text{indicates text missing or illegible when filed}$$

The ratio of MI and M equals to the magnetic field flux changes B1/B0. Utilizing the equations given above, calculated was B1/B0 at 300 kPa using different σ and gets the result of 0.993, 0.975 and 0.955 when σ is 0.05, 0.1 and 0.15, respectively. The magnetic field flux variation obtained using the particle rotation model did not agree with the experimental results indicating that dipole-dipole interaction may have significant influence and need to be considered.

Further developed was a wavy chain model to include the influence of dipole-dipole interaction in the GMMC layer. Previous literature has shown that particle-chain can form in magnetorheological elastomer during the application of external magnetic field and the chain structure is often wavy instead of straight (32, 33). An example GMMC layer shares some-common points with previously studied magnetorheological elastomer. Therefore, it is reasonable to assume that wavy chains are formed during the impulse magnetization process in the present system. Embodiments consider a zigzag chain model as shown in FIGS. 2D and 2F in a two-dimensional assumption. Further assumed was that all the magnetic domain has a vertical dipole moment m which is reasonable because of the system symmetry. Then the magnetic interaction energy per particle can be calculated as below,

$${U}_{int} = \frac{\text{?}}{\text{?}} ( \frac{1}{2} f ( \frac{\text{?}}{\text{?}} ) - 0.1503 ) \qquad ( 5 )$$

$$\text{?} \text{indicates text missing or illegible when filed}$$

where λ is the stretch in the compress direction, b and h are parameters characterizing the structure of wavy chain (FIG. 2F), and

$$f ( \frac{\text{?}}{\text{?}} ) = {\sum }_{n = 1}^{\text{?}} ( {( \frac{\text{?}}{\text{?}} )}^{2} - 2 {( 2 n - 1 )}^{2} ) {( {( \frac{b}{{\text{?}}^{\text{?}}} )}^{2} + {( 2 n - 1 )}^{2} )}^{\text{?}}$$

$$\text{?} \text{indicates text missing or illegible when filed}$$

The situation of λ=1 represents no deformation of the GMMC layer. The value of f(x) with different n and x has been given in FIG. 11. It can be seen that f(x) did not change when n increases from 200000 to 20000000. As a result, embodiments use f(x) of n=2000000 for subsequent calculations.

Besides the interaction energy, the magnetic dipole itself also has magnetic energy as below,

$${U}_{d} = \frac{1}{2} \frac{{B}_{r}^{2}}{{μ}_{0}} \frac{4}{3} π {r}^{3} \qquad ( 6 )$$

Where Br is the remnant magnetic field flux of the magnetic dipole after impulse magnetization, r is the radials of the magnetic dipole. Then the total energy of a magnetic dipole U can be written as U=Ud+Uint. The total energy in a small volume fraction can then be written as W=NU. For a magnetic spherical particle in a weak magnetic form, its magnetic moment m is defined as below,

$$m = 4 π {r}^{3} \frac{{B}_{r}}{{μ}_{0}} \qquad ( 7 )$$

Combining above equations yields

$$W = \frac{N}{2} \text{?} \frac{4}{3} π {r}^{3} + \frac{N 16 π {r}^{6}}{{λ}^{2} {h}^{3}} \frac{{B}_{r}^{2}}{{μ}_{0}} ( \frac{1}{2} f ( \frac{b}{h λ \text{?}} ) - 0.1503 ) \qquad ( 8 )$$

$$\text{?} \text{indicates text missing or illegible when filed}$$

Therefore, the magnetic field in a small volume fraction can be approximated by the equation below.

$$H = \frac{8 W}{8 {B}_{r}} = N \frac{{B}_{r}}{{μ}_{0}} \frac{4}{3} π {r}^{3} + \frac{N 32 π r \text{?}}{{λ}^{2} {h}^{3}} \frac{{B}_{r}}{{μ}_{0}} ( \frac{1}{2} f ( \frac{b}{h λ \text{?}} ) - 0.1503 ) \qquad ( 9 )$$

$$\text{?} \text{indicates text missing or illegible when filed}$$

As demonstrated in equation 2, the magnetic flux density is only determined by the magnetization in a small volume fraction of the GMMC layer. Since the magnetization is proportional to the magnetic field of the small volume, one could use the ratio of the magnetic field in a small volume fraction in different compressed states to evaluate the magnetic flux density changes of the GMMC layer in an ideal situation. The stretch λ in the compressive direction can be further transformed into compressive stress s using a non-compressible Neo-Hookean model as shown below,

$$\text{?} = G ( λ - \frac{1}{\text{?}} ) \qquad ( 10 )$$

$$\text{?} \text{indicates text missing or illegible when filed}$$

Combining equation 9 and 10, it was possible to obtain the evolution of B1/B0 with applied mechanical stress s and compared to experimental results if the b, r, h is determined. For the present NdFeB microparticles, the radius r has an average value of 12.5 μm. The b and h were then measured to be 43.5 μm. As displayed in FIG. 2G, the model and experimental observation agrees well with each other indicating the effectiveness of the wavy chain model.

It should be noted that while the wave chain model accurately captured the deceased magnetic field flux of GMMC layer under compressive stress, it is based on a lot of assumptions and simplifications. Additionally, the model cannot explain the larger magnetic field flux decrease in the corner of the GMMC layer which is a non-ideal situation. A more sophisticated and accurate model is still required to better explain and predict the magnetic behavior of GMMC layer under various mechanical deformations. From a more physical viewpoint, it is hypothesized that the magnetic anisotropy may still play a role in the giant magnetoelastic effect of the GMMC layer. NdFeB particles are expected to be multicrystalline and therefore have multiple easy axes. In the original state, the external impulse magnetization aligned the magnetic moment of NdFeB particles in a single direction. In the compressed state, pressure changed the dipole-dipole interaction of each NdFeB particle. Because the magnetic system is always tending to decrease its overall magnetic potential energy, the dipole-dipole interaction would cause the magnetic moment of NdFeB deviate from its original direction and even jumping to another easy axis. When the compressive pressure is released, the dipole-dipole interaction of each NdFeB microparticle reverses to its original state in which alignment of the NdFeB magnetic moment lowers the overall magnetic energy of the system. As result, the NdFeB magnetic moments realigned and the GMMC layer demonstrates a higher external magnetic flux density.

### 2. Combining Effect for Ultrasound Energy Harvesting

In the past decades, ultrasounds have been universally used in clinical applications for non-invasive tissue imaging. Recently, ultrasound has also attracted increasing interests as a wireless powering source for implantable medical devices to eliminate the bulky and intrusive batteries because of its biosafety, high directivity, deep penetration length and bi-directional communication capability. Piezoelectric effect and triboelectric effect-based energy harvesters have been studied as implantable power generation sources under ultrasound excitation. However, encapsulations are required for these devices to prevent the adverse effect of body fluid and hazard elements, which limited both their performance and long-term usage. In contrast, as demonstrated above, soft MEG has outstanding electric output and waterproof ability making it a suitable candidate as implantable ultrasound energy harvester without encapsulation.

The present implantable soft MEG is composed of a GMMC layer of 4 cm×4 cm and a MI layer with 100 soft copper cycles as illustrated in FIG. 25. A porcine tissue (FIG. 26) was used to mimic human skin because of their similar composition and biostructure. Since the soft MEG contains an MI layer and the ultrasound tip includes a piezoelectric transducer with alternating current input. The MI layer may receive the transmitted electromagnetic energy wirelessly in a direct manner. In order to validate the origin of output electric signal from the ultrasound excitation, first performed was a control experiment comparing the output short-circuit current (Isc) of a MEG with and without the GMMC layer. The result is demonstrated in FIG. 27. It is clear that without a GMMC layer, the Isc of the device is around 0.2 mA, much smaller than the 0.8 mA obtained with a GMMC layer. This result confirms that the majority of the electric output arises from the mechanical movement and deformation of the GMMC layer under the 20 kHz ultrasound excitation. It is worth noting that there exist some high current peaks of 0.6 mA in FIG. 27A which can be ascribed leaked current of the inverter during frequency conversion inside the US tip.

Also investigated were the parameters including the composition of GMMC layer and the gap between GMMC layer and MI layer to optimize the structure of the present soft MEG for better output. As depicted in FIG. 25, the gap is introduced by a laser-cut polystyrene frame with a thickness of 1.2 mm. The result of the MEG output with and without air gap is demonstrated in FIG. 28. It is clear that the output of the MEG with an air gap is higher and more regular compared to the soft MEG without an air gap. Therefore, in the optimization of the GMMC layer, soft MEG configuration with an air gap is adopted. FIG. 29 showed the output Isc of the soft MEG with different compositions and different NdFeB particles under the same ultrasound excitation condition. Two trends can be concluded based on the result. First is that the output of MEG is increasing with increasing thickness of the GMMC layer which can be ascribed to a stronger magnetic field. Second is the output of soft MEG with 5 μm NdFeB particles is stronger than that of soft MEG with 25 μm NdFeB particles. The stronger output of MEG with 5 μm NdFeB particles can be explained also by the stronger magnetic field. However, the influence of NdFeB particle vibration inside the polymer matrix cannot be ignored and point to an opportunity to further optimize the performance of implantable MEG in the future.

Since the 1800 μm thick GMMC layer with 5 μm NdFeB particles deliver the highest Isc as demonstrated in FIG. 29. Embodiments use this MEG configuration to further study the influence of implanted depth and US output to the implantable MEG in FIG. 4 and FIGS. 29-31. While the output summary is demonstrated in FIG. 4 in the main text, FIGS. 29-31 display the detailed output waveforms of the implantable MEG. It can be seen that all the waveforms are sinusoidal at the same frequency of the US source agreeing with previous report. The output of MEG is significantly decreased with increasing implanted depth and decreasing US power output.

Overall, demonstrated was the feasibility of MEG as an implantable power generator to harvest US at 20 kHz. Optimized was the structure of the implantable MEG and obtained the best output Isc of around 1 mA in an ex vivo experiment using porcine tissue as the ultrasound transmission medium.

### 3. Soft MEG for Cardiovascular Health Monitoring.

When the heart contracts, it generates a pulse wave that travels through human's circulatory system. Pulse wave is a combined result of heart, artery functions and blood flowing and contains comprehensive information about human cardiovascular system (CS). Many principal markers of CS system such as pulse wave velocity (PWV), heart rate, stiffness index (SI) and left ventricular ejection time (LVET) can be extracted utilizing high-sensitivity pulse wave sensor to make appropriate recommendation for clinical practice (34). Benefiting from the softness and high-sensitivity, the soft MEG can be used to detect radial pulse with fine structures in real time and continuously. FIG. 34 depicts a typical arterial pulse waveform containing systolic peak (P1), reflected peak (P2), dicrotic notch and diastolic peak (P3).

The first important parameter estimating the heart condition is the heart rate. Heart rate can be influenced by many factors such as body position, exercise, medication use and so on. Typically, the heart rate of a healthy adult is from 60 to 100 beats per minutes (bpm). FIG. 35 calculates the heart rate measured by the MEG under water which is around 73 bpm.

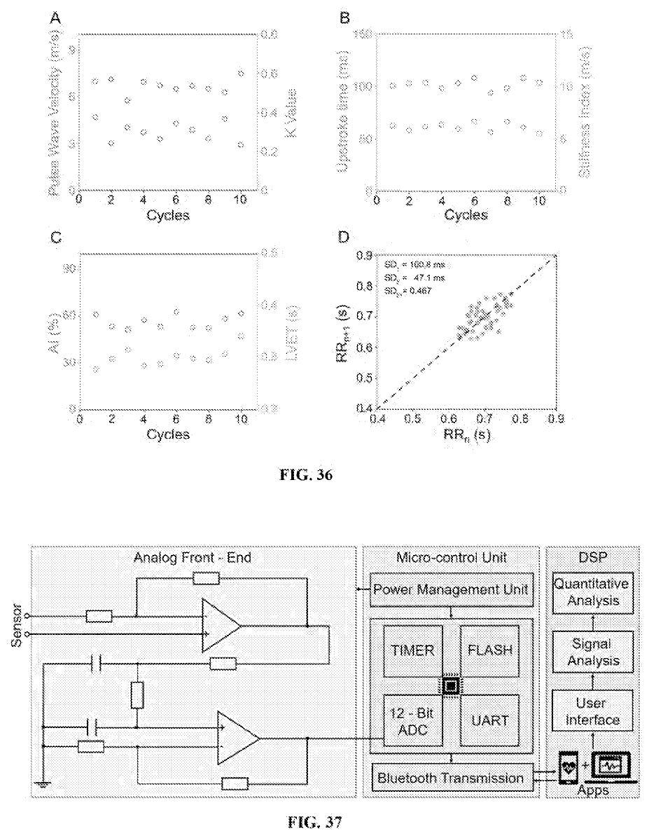

Pulse wave velocity (PWV), the speed of the pulse wave, is proportional to the square root of the incremental elastic modulus of the vessel wall through Moens-Korteweg equation. Therefore, it is considered the clinical “gold standard” measurement to determine arterial stiffness. It is also supported by large amount of epidemiological evidence for its prognostic significance of cardiovascular events. PWV is obtained using a single-point wave separation method and the equation is given below,

$$PWV = 0.8 \times \frac{2 Δ L}{RWTT} \qquad ( 11 )$$

where RWTT is the time interval between P2 and P1 in FIG. 34, ΔL is the distance between jugulum and pubic symphysis. Typically, RWTT decreases and PWV increases with increasing arterial stiffness. PWV of health human beings from 30 to 39 year old has an average value of 6.5 m/s and normal distribution between 3.8 and 9.2 m/s (±2 SD), FIG. 36A demonstrates a PWV value measured by the soft MEG in the range of 5.77 to 7.49 m/s indicating a healthy status of the tester.

K value is a parameter estimating the mean arterial blood pressure. It can be calculated using the following equations,

$$K = \frac{{P}_{m} - {P}_{v}}{{P}_{1} - {P}_{0}} \qquad ( 12 )$$

$${P}_{m} = \text{?} {\int }_{0}^{t} P ( t ) dt \qquad ( 13 )$$

$$\text{?} \text{indicates text missing or illegible when filed}$$

where t is the pulse length, P0 is the diastolic pressure, P1 is the systolic peak pressure and Pm is the mean pressure. Typically, K value can be classified into 4 categories: “low resistance type” (K<0.35), “medium resistance type” (0.35<K<0.40), “high resistance type” (0.40<K<0.45), and “ultrahigh resistance type” (0.45<K<0.5), FIG. 36A shows an average K value of 0.302 belonging to the low resistance type, The small K value imply a healthy body status of a young adult consistent with previous literature report.

Upstroke time is the transit time from the nadir to systolic peak of the waveform of pulse. It is getting more and more attentions as a useful cardiovascular marker for detecting artery diseases. For a healthy person, the upstroke time is less than 180 ms. FIG. 36B demonstrate the measured upstroke time to be around 100 ms, well within the healthy range of the reference value.

Stiffness index (SI) is another parameter evaluating the artery stiffness and measure of PWV in large arteries. It is calculated as follows,

$$SI = \frac{\text{?}}{PPT} \qquad ( 14 )$$

$$\text{?} \text{indicates text missing or illegible when filed}$$

where PPT is the time interval between the diastolic peak and systolic peak, H is the height of the subject. SI in FIG. 36B ranges from 5.5 m/s to 6.7 m/s, close to the value of PWV in FIG. 34A, indicating a well correlation relationship between each other.

Augmentation index (AI) is defined as the ratio between reflected peak P2 and systolic peak P1. It is closely related to the arterial stiffness and proposed to reliably predict adverse cardiovascular events. FIG. 36C presents AI of 10 pulse cycles which are all around 60% and are close to the value of investigated persons without cardiovascular diseases.

LVET measures the ability of heart to produce contractile force. FIG. 36C plots the LVET of the subject which is defined as the time interval between the dicrotic notch and the beginning of systolic stage. The normal value of LVET of a healthy cardiovascular system is 260-340 ms. The average LVET of the subject is calculated to be 301 ms again denoting a healthy cardiovascular condition of the subject.

Heart rate variety, also known as RR interval, is important independent parameter evaluating the heart condition. It can be assessed by a Poincare plot which plots each RR interval against the next interval. FIG. 36D displays the Poincare plot of the subject. It can be seen that all the points form a comet shape with an ellipse contour. Through fitting technique, the ellipse width, ellipse length and their ratio which are referred as SD1, SD2 and SD12=SD1/SD2 can be determined. It is evidenced that when SD12 is larger than 0.55, there is an increasing risk of high cardiac death. The SD12 of the subject is fitted to be 0.467 suggesting a healthy heart state.

### B. Giant Magnetoelastic Effect in a Soft Fiber

### B. Summary

According to a second aspect, the present embodiments relate generally to methods and apparatuses for obtaining a giant magnetoelastic effect in a 1D soft microfiber with up to 8.4 times enhancement of magnetomechanical coupling comparing to that in the traditional bulky metal alloys. To understand it, established is a wavy chain analytical model based on the magnetic dipole-dipole interaction, which is consistent with the experimental observation. The discovery is explored further in the microfiber and coupled with magnetic induction to develop an origami-programmed textile magnetoelastic generator (MEG), which paves a new way for biomechanical-to-electrical energy conversion with unprecedented high short-circuit current density of 0.63 mA cm⁻² and ultralow internal impedance of 180Ω, corresponding to three orders of magnitude improvement than its textile counterparts. Moreover, resulting from unique working principle, the textile MEG is intrinsically waterproof, which was demonstrated to sensitively convert the arterial pulse into electrical signals with heavy perspiration, even in underwater situation without reliance of encapsulations. Based on a built-in algorithm, a customized cellphone application featuring one-click sharing and database-driven diagnosis was further designed for wearable self-powered cardiovascular parameters measurement. It is foreseen that the discovery of giant magnetoelastic effect in 1D microfibers is creating a compelling platform to be further integrated with a number of other effects, including but not limited to magnetic induction, magneto-optic effect, and magnetocaloric effect for developing high-performance soft-matter technologies.

### B. Introduction

Magnetoelastic effects are found in rigid and 3D metal alloys such as Fe₁₋ₓCoₓ (Yamaura, S.-i., Nakajima, T., Satoh, T., Ebata, T. & Furuya, Y. Magnetostriction of heavily deformed Fe—Co binary alloys prepared by forging and cold rolling. Mater. Sci. Eng. B 193, 121-129 (2015)), TbₓDy₁₋ₓFe₂ (Terfenol-D) (Su, Q., Morillo, J., Wen, Y. & Wuttig, M. Young's modulus of amorphous Terfenol-D thin films. J. Appl. Phys. 80, 3604-3606 (1996)), and GaₓFe₁₋ₓ (Galfenol) (Datta, S., Atulasimha, J., Mudivarthi, C. & Flatau, A. B. Stress and magnetic field-dependent Young's modulus in single crystal iron-gallium alloys. J. Magn. Magn. Mater. 322, 2135-2144 (2010)), which are usually used for civil engineering for building vibration control with an applied magnetic field (Ausanio, G., et al. Magnetoelastic sensor application in civil buildings monitoring. Sens. Actuator A Phys. 123-124, 290-295 (2005)). This effect has been restrained from the field of soft-matter electronics for three reasons: (1) the rigid metal alloys hold 6 orders higher mechanical modulus than human bodies, (2) the required mechanical stress for the magnetoelastic alloys is beyond the range of biomechanical stress, and (3) they rely on external magnetic fields resulting in a bulky structure.

Textiles (Chen, G., Li, Y., Bick, M. & Chen, J. Smart textiles for electricity generation. Chem. Rev. 120, 3668-3720 (2020); Alberghini, M., et al. Sustainable polyethylene fabrics with engineered moisture transport for passive cooling. Nat. Sustain. https://doi.org/10.1038/s41893-021-00688-5 (2021)), one of the earliest human inventions (Hsu, P.-C. & Li, X. Photon-engineered radiative cooling textiles. Science 370, 784-785 (2020)), have become an indispensable part of our lives owing to their unique properties, such as light weight, touching softness, and inherent breathability (Peng, X., et al. A breathable, biodegradable, antibacterial, and self-powered electronic skin based on all-nanofiber triboelectric nanogenerators. Sci. Adv. 6, eaba9624 (2020); Dong, K., et al. Shape adaptable and highly resilient 3D braided triboelectric nanogenerators as E-textiles for power and sensing. Nat. Commun. 11, 2868 (2020); Fan, W., et al. Machine-knitted washable sensor array textile for precise epidermal physiological signal monitoring. Sci. Adv. 6, eaay2840 (2020)). Merging textiles and electronics is a compelling approach to realize smart textiles with additional values while maintaining the wearing comfort. Biomechanical motions are clean and renewable energy sources (Chen, J., et al. Micro-cable structured textile for simultaneously harvesting solar and mechanical energy. Nat. Energy 1, 16138 (2016)). Fiber-based textiles can effectively accommodate the body motions induced complex deformation for electricity generation, which is an essential pathway to build up human-centered self-powered bioelectronics for energy, sensing and therapeutics (Xu, C., Yang, Y. & Gao, W. Skin-interfaced sensors in digital medicine: from materials to applications. Matter 2, 1414-1445 (2020); Zhou, Z., et al. Sign-to-speech translation using machine-learning-assisted stretchable sensor arrays. Nat. Electron. 3, 571-578 (2020)). Currently, the widely adopted biomechanical energy harvesting textiles that based on triboelectric effect (Pu, X., et al. A self-charging power unit by integration of a textile triboelectric nanogenerator and a flexible lithium-ion battery for wearable electronics. Adv. Mater. 27, 2472-2478 (2015)) and piezoelectric effect (Ghosh, S. K. & Mandal, D. Synergistically enhanced piezoelectric output in highly aligned 1D polymer nanofibers integrated all-fiber nanogenerator for wearable nano-tactile sensor. Nano Energy 53, 245-257 (2018); Qin, Y., Wang, X. & Wang, Z. L. Microfibre-nanowire hybrid structure for energy scavenging. Nature 451, 809-813 (2008); Anwar, S., et al. Piezoelectric nylon-11 fibers for electronic textiles, energy harvesting and sensing. Adv. Funct. Mater. 31, 2004326 (2020); Guan, X., Xu, B. & Gong, J. Hierarchically architected polydopamine modified BaTiO3@P (VDF-TrFE) nanocomposite fiber mats for flexible piezoelectric nanogenerators and self-powered sensors. Nano Energy 70, 104516 (2020)) display low current density (in the order of hundreds nA cm⁻²), and high internal impedance (in the order of several megaohms), which are ascribable to their capacitive electricity generation principle by manipulating the electric dipoles at the materials interfaces (Fan, F.-R., Tian, Z.-Q & Wang, Z. L. Flexible triboelectric generator. Nano Energy 1, 328-334 (2012). More importantly, their electrical output performance is vulnerable to ambient humidity caused by perspiration and fluidic environment of the human body, which severely limits their practical on-body applications. An encapsulation layer would enhance the device humidity resistance. However, it usually compromises their electric output performance, undermine textile breathability and wearability (Zheng, Q., et al. In vivo self-powered wireless cardiac monitoring via implantable triboelectric nanogenerator. ACS Nano 10, 6510-6518 (2016)).

As set forth above, the present Applicant discovered the giant magnetoelastic effect arising from magnetic dipole alignment in soft microfibers, which demonstrated up to 8.4 times stronger magnetomechanical coupling than that observed in the conventional rigid metal alloys. Via manipulating the magnetic dipole-dipole interaction, the discovered giant magnetoelastic effect in the soft microfibers shows a collection of compelling advantages: (1) Giant magnetoelastic effect is realized in soft microfibers with Young's modulus around 630 kPa, which is mechanically compatible with human body. (2) The applied pressure that needs to deform the 1D soft microfiber is within the range of 450 kPa and obtainable by human daily activities. (3) It requires no external magnetic field. These features endow the 1D soft microfibers wide-range of applicability in building up soft-matter technologies. To demonstrate, an origami-programmed textile magnetoelastic generator (MEG) is developed relying on a two-step conversion mechanism that couples giant magnetoelastic effect and magnetic induction. It shows unprecedented performance in converting the biomechanical activities into electrical energy. A short-circuit current density of 0.63 mA cm⁻² and an internal impedance of 180Ω was achieved, corresponding to three orders of magnitude improvement than other textile counterparts for biomechanical energy conversion. Since magnetic fields can penetrate water with negligible intensity loss, the textile MEG is intrinsically humidity-resistant without additional encapsulation. The wearable textile MEG was also applied to convert the arterial pulse into electrical signals under the circumstance of heavy body perspiration for self-powered cardiovascular parameter measurement. A customized cellphone application (APP) based on built-in algorithm was also developed for one-click health data sharing and data-driven diagnosis. With features like intrinsic humidity-resistance, high current, and low internal resistance, it is foreseen that the discovery of giant magnetoelastic effect in 1D microfibers could be used as building blocks for soft-matter electronics with wide-range of applications in energy, medical, robotics, and artificial perception fields.

### B. Design of Soft Magnetic Microfibers

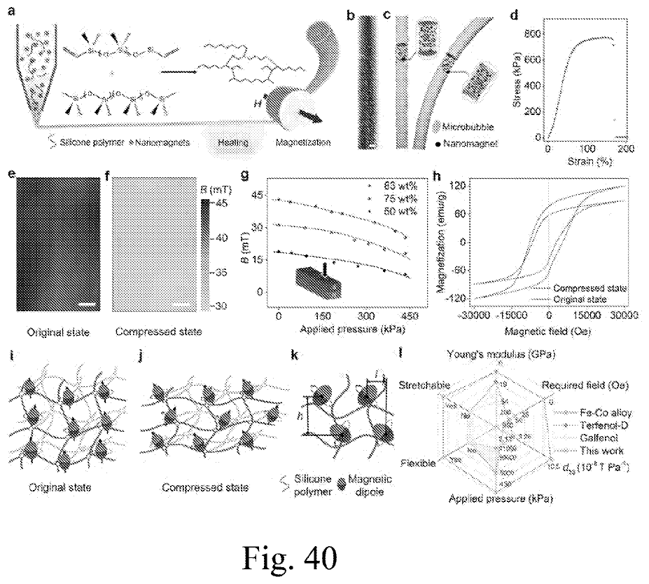

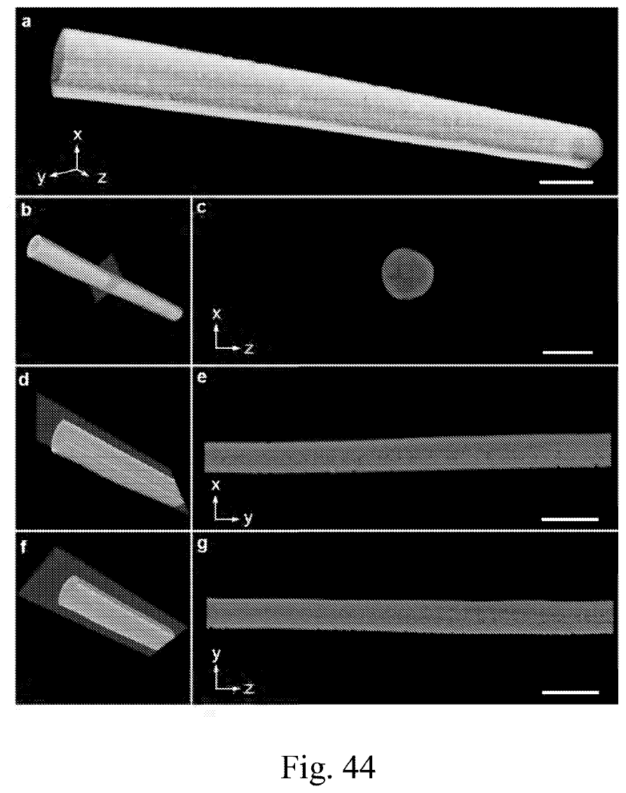

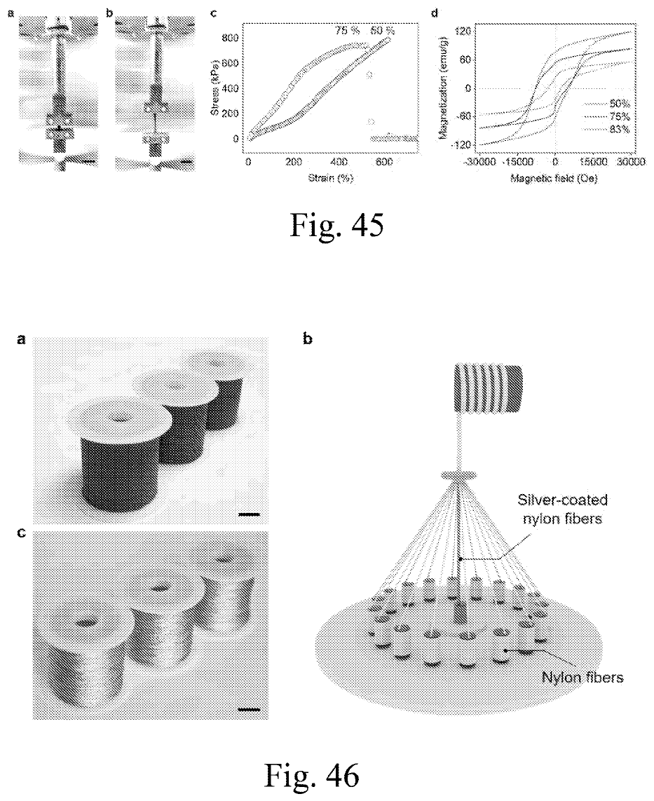

The fabrication procedure of the 1D soft microfibers with giant magnetoelastic effect was schematically illustrated in FIG. 40a. Solid nanomagnets were uniformly dispersed and embedded in the highly viscous liquid silicone polymer. Air microbubbles were systematically introduced into the composite during mechanical stirring to form a three-phase mixture, which will further go through an extrusion processing via an adjustable nozzle. The soft magnetic microfibers with controllable diameters were obtained by crosslinking the three-phase mixture during the heating process. As demonstrated in the cross-section view of the scanning electron microscope (SEM) image, the nanomagnets were evenly dispersed in the soft magnetic microfibers (FIG. 40b). Microbubbles play a critical role of bringing porosities into the soft magnetic microfibers. On the one hand, the outmost microbubbles were broken to form surface roughness on the microfibers, improving the softness, touching comfort and stretchability. On the other hand, it helps reduce the overall fiber density. Micro computed tomography (Micro-CT) was employed to characterize the fiber internal structure. As shown in the FIG. 40b, there are uniformly distributed microbubbles induced nanoscale-to-microscale cavities in the soft magnetic microfibers, which is consistent with all the three cross-section views shown in FIG. 44. This observation is also justified by the overall morphology of the magnetic microfibers showing in a dynamic 3D model created by Micro-CT. Owing to the designed microbubble cavities, the soft magnetic microfibers could be effectively deformed under gentle bending and compressing (FIG. 40c). As shown in FIGS. 45a-c, by controlling the magnetic concentrations, the magnetic microfibers show different mechanical properties. Hence, the 83 wt % soft magnetic microfiber with a stretchability up to 180% and a Young's modulus of 630 kPa was chosen (FIG. 40d), for it holds similar mechanical properties with human tissues and can ensure its adaptability to human skin (Pailler-Mattei, C., Bec, S. & Zahouani, H. In vivo measurements of the elastic mechanical properties of human skin by indentation tests. Med. Eng. Phys. 30, 599-606 (2008); Kováčik, J. Correlation between Young's modulus and porosity in porous materials. J. Mater, Sci. Lett. 18, 1007-1010 (1999)). Apart from the advantages in mechanical properties, the 83 wt % soft magnetic microfibers have larger coercive field of 7.89 kOe and remnant magnetization of 76 emu g⁻¹ than that of magnetic microfibers with 75 wt % and 50 wt % magnetic concentrations (FIG. 45d). Moreover, it is worth noting that the soft magnetic microfibers could also be massively produced. Three spools of microfibers could be obtained via one-time machine extrusion (FIG. 46a).

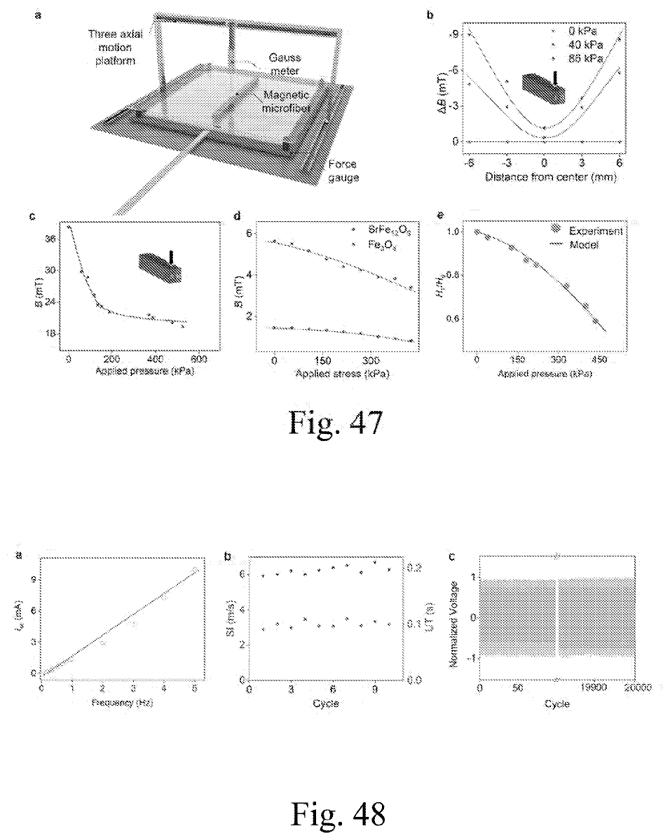

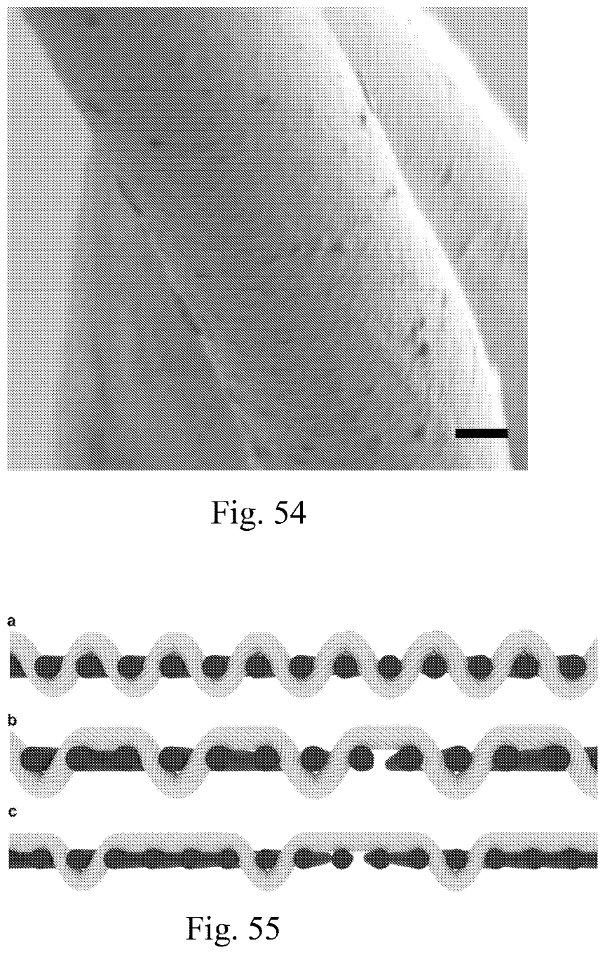

To reorient the magnetic dipoles in the microfibers, an adjustable impulse fields H was employed for magnetization. The magnetic dipoles were aligned in the soft polymer matrix with uniform polarity. In order to investigate the magnetic field variation under mechanical deformation, a three-axial motion platform was established to visualize the magnetic field variation under different applied stress, as schematically illustrated in FIG. 47a. And the magnetic flux density on the south pole, north pole, and side surface of the soft microfibers were systematically investigated. FIG. 40e shows the magnetic flux density mapping on the south pole of the soft microfiber in its initial state, and FIG. 40f shows the mapping image in its compressed state under a pressure of 300 kPa. A comparative study indicates that the magnetic flux density decreased in both the edge and the middle of the microfiber under applied stress. A similar trend was also observed for the magnetic flux density mapping on the north pole of the 1D microfiber (FIG. 45). Also investigated were the values of magnetic field variation with different nanomagnet concentrations. As shown in FIG. 40g, under a constant stress, soft magnetic microfibers with 83 wt % nanomagnet concentration have the largest values of magnetic field variation of 18 mT than that with 75 wt % (13 mT) and 50 wt % (11 mT) nanomagnet concentrations. As a result, a maximum magnetomechanical coupling factor of 8.28×10⁻⁸ T Pa⁻¹ was derived from magnetic field variation of the 1D microfibers, corresponding to a 6.6-time enhancement comparing to that reported in the traditional bulky metal alloys. As for the side surface, the magnetic flux density mapping images are shown in FIG. 54, and the magnetic field variation (ΔB) data are plotted in FIG. 47b. The AB are larger in the edge than that in the middle of the magnetic microfiber under a fixed applied pressure. And a maximum magnetomechanical coupling factor of 1.05×10⁻⁷ T Pa⁻¹ was derived from the edge of the side surface, corresponding to an 8.4-time enhancement comparing to that reported in the traditional bulky metal alloys (FIG. 47c). Furthermore, magnetic hysteresis loop of the soft magnetic microfiber in original state and compressed state are tested. As shown in FIG. 40h, both the remnant magnetization and coercive field of the magnetic microfiber decreased in compressed state, which may due to the rearrangement of the nanomagnets inside the soft magnetic microfibers. This result is consistent with the decreasing magnetic flux density on the surface of the magnetic microfibers.

More importantly, verified was a giant magnetoelastic effect in different ferromagnetic materials and it was found that magnetoelastic effect arising from magnetic dipole: alignment in soft system is a universal phenomenon. Soft magnetic microfibers made of SrFe₁₂O₉ and Fe₃O₄ also demonstrated magnetic field decrease under mechanical deformation, which is similar to that made by NdFeB nanomagnets (FIG. 47d). In order to compare their mechanical and magnetic properties, summarized are their magnetomechanical coefficient and Young's modulus in Table B-1.

TABLE B-1

| | | | | Required | Magneto- | Applied |
|---|---|---|---|---|---|---|
| | | | | external | mechanical | pressure |
| Material | | Young's | | magnetic | coupling | range |
| category | | modulus | | field (Oe) | factor (T/Pa) | (kPa) |
| Fe—Co alloy | | 200 | GPa^{a} | 25^{c} | 1.25 × 10^{-8a} | 5000- |
| | | | | | | 70000 |
| Co | | 209 | GPa^{a} | 10^{c} | 2.45 × 10^{-8a} | — |
| Terfenol-D | | 19 | GPa^{a} | 950^{c} | 3.26 × 10^{-8a} | 11000- |
| | | | | | | 41000 |
| Galfenol | | 54 | Gpa^{a} | 50^{c} | 2.13 × 10^{-8a} | 10000- |
| | | | | | | 50000 |
| MEG | NdFeB^{e} | 629.76 | kPa^{b} | 0 | 1.05 × 10^{-7 d} | 0-450 |

| | | | | |
|---|---|---|---|---|
| Fe₃O₄^{e} | 303.12 | 0 | 2.96 × 10⁻⁹ | 0-450 |
| SrFe₁₂O₁₉^{e} | 834.48 | 0 | 8.37 × 10⁻⁹ | 0-450 |

^{a}Estimated value.
^{b}Soft magnetic fiber with 83 wt % concentration.
^{c}Calculated based half of the saturation.
^{d} Calculated based maximum value on side surface.
^{e}Made by 83 wt % of magnetic concentrations.

### B. Wavy Chain Analytical Model

To explain the ultra-strong magnetomechanical coupling in the soft magnetic. microfibers, a wavy chain analytical model was established based on the magnetic dipole-dipole interactions. As illustrated in FIG. 40i, in its original state, the nanomagnets forms wavy chains in the polymer matrix under impulse magnetic fields. When a compressive stress is applied to the microfiber, the corresponding shape deformation leads to the distance and orientation variation of the magnetic dipoles (FIG. 40j). As a result, the dipole-dipole interaction inside the wavy chain is altered associated with an observation of the external magnetic field decrease. Quantitively, the magnetic field variation in response to applied mechanical pressure can be evaluated by H1/H0, which can be expressed as a function of principle stretch λ based on the wavy chain model as below,

$${H}_{1} / {H}_{0} = ( 1 + 24 β ( \frac{l}{h λ \text{?}} ) \cdot {( \frac{r}{dh} )}^{3} ) / ( 1 + 24 β ( \frac{l}{h} ) \cdot {( \frac{r}{h} )}^{3} ) \qquad ( 1 )$$

$$\text{?} \text{indicates text missing or illegible when filed}$$

Where H1 and H0 represent the magnetic field with and without applied mechanical pressure, respectively, and r is the radius of the nanomagnet. As illustrated in FIGS. 40k, h and l denote the vertical and horizontal distances between two adjacent magnetic dipoles in the wavy chain, respectively. β(x) is the dipole alignment factor describing the magnetic interaction energy that all other magnetic dipoles contribute to a single dipole in the wavy chain. The derivation of the β(x) is detailed in Supplementary Note 1. The magnetic field variation H1/H0 is then linked to the compressive stress s through an incompressible Neo-Hookean material model with the following equation,

$$\text{?} = G ( λ - 1 / {λ}^{2} ) \qquad ( 2 )$$

$$\text{?} \text{indicates text missing or illegible when filed}$$

where G is shear modulus of the soft magnetic microfiber. With estimated value of r=2.5 μm, h=7.8 μm and l=9.36 μm in the soft magnetic microfiber, the wavy chain model accurately captures its magnetic field variation in response to the compressive stress changing from 0 to 450 kPa, which is well consistent with the experimental observation (FIG. 47e). With both experimental results and theoretical verification, it is clear that the compressive stress on the order of 100 kPa was enough to generate a significant magnetic field variation up to ˜18 mT in the 1D soft magnetic microfiber.

Besides the magnetomechanical coupling analysis, embodiments took a further step to comprehensively compare the 1D soft microfibers with Fe—Co alloys, Terfenol-D and Galfenol in other parameters, which were reported to show strongest magnetoelastic effect, as the results summarized in FIG. 40l. Towards building up soft matter technologies, the 1D soft magnetic microfibers show great advantages over the two metal alloys in all the six performance indexes, including magnetomechanical coupling factor d₃₃ (T/Pa), Young's modulus (kPa), flexibility, stretchability, applied stress (kPa), and required magnetic field (A/m). Specifically, (1) The maximum magnetomechanical coupling factor (das) of soft microfibers shows up to 8.4-time enhancement than that in the metal alloys. (2) The soft microfibers are flexible and stretchable with Young's modulus of 630 kPa, while metal alloys are bulky and rigid with Young's modulus of up to 200 GPa. (3) The applied pressure on the soft microfibers is below. 450 kPa, which is much lower than the typical value (5-11 MPa) of conventional metal alloys (4) The soft microfiber requires no external magnetic field to perform mechanical-to-magnetic conversation, while metal alloy demands external magnetic field up to 950 Oe via external permanent magnets or bulky electromagnets. With a collection of these compelling features, the developed 1D soft microfibers with giant magnetoelastic effect shows great potential in mechanical-to-magnetic conversion in a soft manner. And it could be further coupled with other effects, including but not limited to magnetic induction, magneto-optic effect, and magnetocaloric effect for wide range of applications in electricity generation, optical regulation and thermal management.

### B. Constructing a Textile MEG

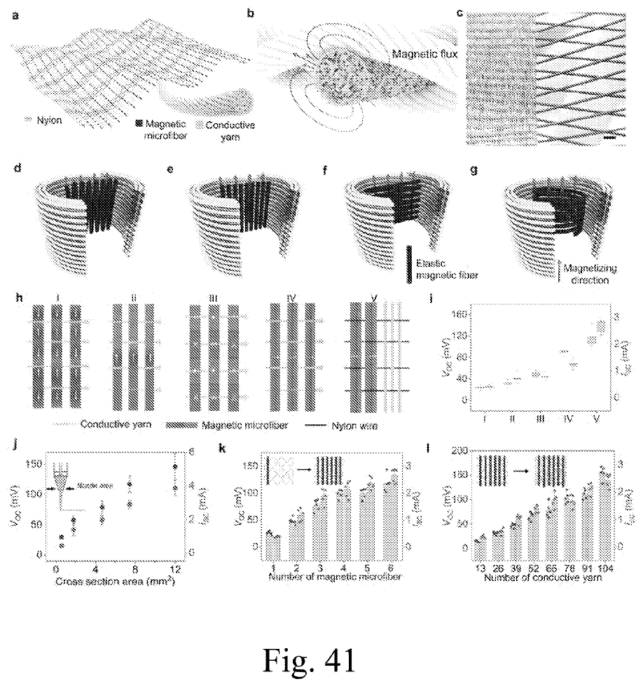

To demonstrate the viability of the discovered giant magnetoelastic effect in 1D soft microfibers, embodiments further coupled it with the magnetic induction to develop a wearable textile MEG as a soft-matter electronic for on body electricity generation. The textile MEG is formed by interlacing the magnetic microfibers with the conductive yarns, as illustrated in FIG. 41a. The conductive yarns were consisted of silver-coated nylon fibers and nylon fibers winded by a multiaxial yarn winding machine (FIG. 46b), and they are amenable to large-scale fabrication (FIG. 46c). The silver coated on the nylon microfiber is very uniform to assure the fiber conductivity, as the SEM image demonstrated in FIG. 54. The electricity generation of the textile MEG in response to applied force is relying on a two-step energy conversion, namely, mechanical-to-magnetic and magnetic-to-electrical conversions. The deformation of soft magnetic microfibers was used to efficiently create magnetic fields variation under mechanical stimulus, which was then converted into electricity by using the conductive yarns based on magnetic induction. Structurally, the conductive yarns are woven with porous magnetic microfibers within the textile MEG, the two-step conversion happens at each yarn intersection when the textile is clapped, shook or folded, as schematically illustrated in FIG. 41b. It is worth noting that the textile MEG could be massively produced by an industrial weaving loom (FIG. 41c).

### B. Parameter Optimization

To achieve the optimized mechanical-to-electrical energy conversion, a number of designing parameters were systematically investigated in the textile MEG. To begin with, in the first step of mechanical-to-magnetic conversion, the magnetization pattern of the soft magnetic microfibers plays an important role in creating efficient magnetic field variation. Thus, magnetic microfibers with different magnetic domain distribution were compared. In order to program the magnetic microfibers with controllable magnetic domain, the microfiber was bent to different origami shapes under a constant magnetizing field (FIGS. 41d-g). The corresponding magnetic domain distributions in the soft microfibers are demonstrated in the FIG. 41h, in which origami pattern of mode I, II, and III were programmed with magnetization direction depicted in FIGS. 41d, 41e, and 41f, respectively, and the origami pattern of mode IV and V are programmed using magnetization direction depicted in FIG. 41g. Their electrical outputs were systematically investigated as shown in FIG. 41i. From mode I to IV, when the magnetic microfibers were vertically interlacing with the conductive yarns, the magnetic domain distribution in mode IV showed the highest electrical output, owing to the uniform domain distribution in the magnetic microfibers. However, when two kinds of yarns are interlacing in parallel with each other shown in mode V, it demonstrated even better electrical performance than mode IV, because it can accommodate more magnetic field variation. Additionally, the cross-sectional area and number of the magnetic microfibers also have great influence on the magnetic field variation in the textile. As illustrated in FIG. 41j, the origami-programmed textile MEG has better electrical output with larger cross-section area of magnetic microfibers, since larger magnetic flux variation can be created in thicker magnetic fibers under mechanical excitation. Besides the fiber diameter, the electrical output of the origami-programmed textile MEG could also be largely boosted with the elevation of the fiber numbers. As shown in FIG. 41k, both the current and voltage increase linearly with the more woven magnetic microfibers.

Furthermore, the number of the conductive yarns was also investigated because it is critical to the second step of magnetic-to-electrical conversion. As demonstrated in FIG. 41l, the electrical output increases linearly with the increase of the conductive yarns. It is understandable with Faraday's law since the number of conductive yarns is directly proportional to the induced voltage. As a result, the electrical output of the textile MEG is designable with the origami patterns, and the number of both magnetic microfibers and conductive yarns.

### B. Origami-Programmed Textile MEG for Energy

Harvesting energy from biomechanical motions with a textile provide a pervasive and convenient energy solution for body-centered electronics while maintaining the wearing comfort. Humidity-resistance is an indispensable feature of on-body applications since sweat glands on human skins can perspire up to 3.5 L hour⁻¹ in humans' daily exercise (Wendt, D., van Loon, L. J. C. & Marken Lichtenbelt, W. D. Thermoregulation during exercise in the heat. Sports Med. 37, 669-682 (2007)). The current triboelectricity and piezoelectricity-based textile biomechanical energy harvesters are not intrinsically waterproof owning to their capacitive electricity generation principle via manipulating the electric dipoles at the materials interfaces. Adding an additional encapsulation layer could achieve certain humidity-resistance at the price of undermining the electric output, and wearability. On the basis of magnetic dipole manipulation, the fundamentally new origami-programmed textile MEG is intrinsically waterproof since the magnetic fields are able to penetrate water with negligible intensity loss.

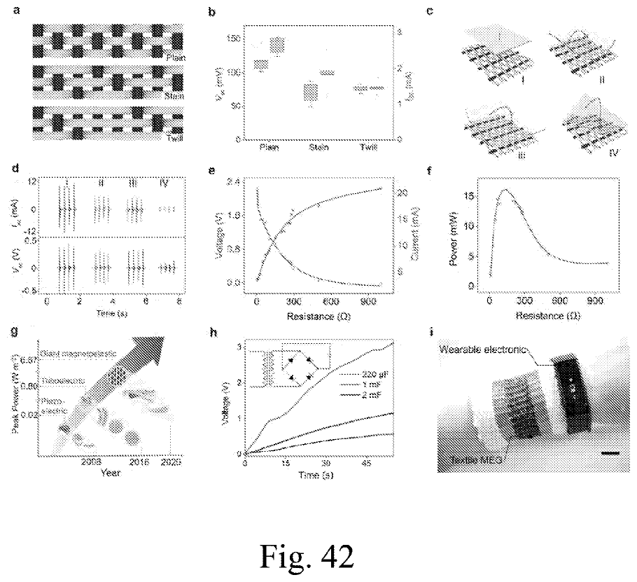

Towards practical application, the weaving pattern of the textile were systematically investigated, because this is a necessary designing parameter for consideration since it shows impact on both the electrical output and the appearance of the textile MEG. As illustrated in FIG. 42a and FIG. 55, three basic weaving patterns, plain, stain and twill are structurally designed and tested. Their corresponding voltage and current outputs were demonstrated in FIG. 42b. The textile with plain-weave pattern has the highest electrical output, followed by that of stain-weave patterned textile, while the twill-weave pattern delivers the lowest electrical output. This is mainly due to their different deformation degree under a fixed mechanical stress. Furthermore, the electricity generation process is also highly related to the mechanical excitation modes. As shown in FIG. 42c, embodiments took the plain-woven structure textile MEG for investigation under four mechanical excitation modes, including clapping the textile with a flat surface (mode I), folding the textile parallelly (mode II) and perpendicularly (mode III) to the central magnetic microfiber. In mode IV, the textile is folded along its diagonal line. As the experimental results displayed in FIG. 42d, the mode I has the best performance followed by mode II, mode III, and mode IV. This observation is also ascribed to the magnetic field variation difference in response to the four mechanical excitation modes, especially the magnetic field variation would be partially canceled in the modes II, III and IV when the textile was folded.

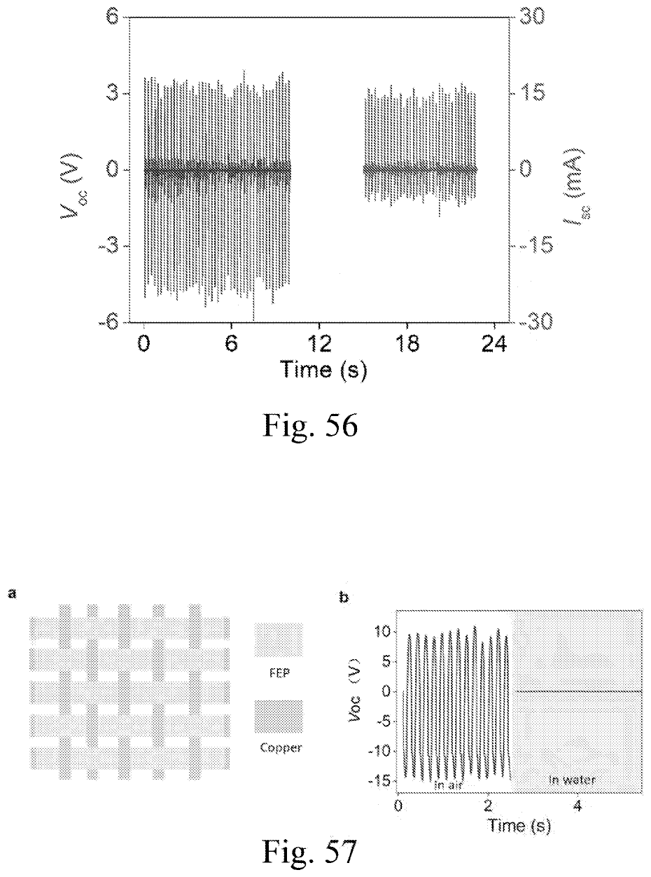

To demonstrate the textile MEG as a sustainable power source, a piece of 4 cm-by-6 cm textile with plain weaving pattern was fabricated and measured. As shown in FIG. 56, when the origami-programmed textile was excited by hand clapping with heavy perspiration, it delivers an ultrahigh short-circuit current of 15 mA, corresponding to a short-circuit current density of 0.63 mA cm⁻². It also shows ultralow in internal impedance of 180Ω. These achievements represent three orders of magnitude improvement than other textile biomechanical energy harvesting technologies in the community, as the comparison summarized in Table B-2. With the size of 4 cm by 6 cm, the textile also delivered a voltage of 4.6 V. It is worth noting that the voltage output could be greatly increased with larger size of the textile. To evaluate the output power of the textile MEG, resistors were employed as the external loads. As shown in FIG. 42e, the current decreases with the elevation of the loading resistances, while the voltage follows a reverse trend. A maximum peak power of 16 mW is obtained at the matched load of 180Ω, corresponding to a power density of 6.67 W m⁻² (FIG. 42f). Comparing to textile biomechanical energy harvesting technologies based on piezoelectric (Zeng, W., et al. Highly durable all-fiber nanogenerator for mechanical energy harvesting. Energy Environ. Sci. 6, 2631-2638 (2013)) or triboelectric (Chen, C., et al. 3D double-faced interlock fabric triboelectric nanogenerator for bio-motion energy harvesting and as self-powered stretching and 3D tactile sensors. Mater. Today 32, 84-93 (2020)) effects, the waterproof textile MEG represents an 8.3 times enhancement in power density (FIG. 42g). To demonstrate the capability of the origami-programmed textile MEG as an intrinsically waterproof power source, the 4 cm-by-6 cm encapsulation-free textile MEG was worn on the sweaty human arm with hand tapping, it was able to charge a 220 μF commercial capacitor to 3 V within 53 seconds (FIG. 42h), and sustainably drove a wearable smart biosensor system for continuously multiple physiological information monitoring (FIG. 42i). Additionally, fabricated and tested was a typical triboelectric effect based biomechanical energy harvesting textile as a comparison since it has a favorable electric performance among their counterparts in the community. Under a same application scenario without encapsulation, its electrical output was adversely affected and rapidly reduced more than three order of magnitudes, as the results shown in FIG. 57. In summary, with features like intrinsic humidity-resistance, high current, and low internal resistance, the developed textile 7 MEGs represent a milestone in the community of biomechanical energy harvesting, and could be potentially applied in medical and miniaturized robotics.

TABLE B-2

| | | | | | Peak power | | Internal | | | |
|---|---|---|---|---|---|---|---|---|---|---|
| Generators | V_{oc}^{a} | | I_{sc}^{b} | | density | | impedance | | Year | Ref |
| Textile PENG | 1-3 | mV | 6.66 | nA cm^{-2c} | 16 | mW m⁻² | 250 | MΩ^{c} | 2008 | 4 |
| Textile PENG | 3.4 | V | 0.70 | μA cm^{-2c} | — | | — | | 2013 | 5 |
| Textile PENG | 6 | V | 0.24 | μA cm^{-2c} | 8.78 | mW m⁻² | 5 | MΩ^{c} | 2020 | 6 |
| Textile PENG | 6 | V | — | | 0.031 | W m⁻² | 500 | KΩ^{c} | 2020 | 7 |
| Textile TENG | 50 | V | 0.16 | μA cm^{-2c} | 0.39 | W m⁻² | 70 | MΩ^{c} | 2015 | 8 |
| Textile TENG | 18 | V | 0.056 | μA cm^{-2c} | 0.80 | W m^{-2c} | 10 | MΩ^{c} | 2016 | 9 |
| Textile MEG | 4.6 | V | 0.63 | mA cm⁻² | 6.67 | W m−2 | 180 | Ω | 2021 | This work |

^{a}V_{oc}: open circuit voltage.
^{b}I_{sc}: short circuit current.
^{c}Estimated value

### B. Origami-Programmed Textile MEGs for Sensing

Arterial pulse measurement is a vital means for assessment of cardiovascular disease (Chen, G., Au, C. & Chen, J. Textile triboelectric nanogenerators for wearable pulse wave monitoring. Trends Biotechnol. https://doi.org/10.1016/j.tibtech.2020.1012.1011 (2021). Continuous and accurate monitoring the arterial pulse signal without influence of perspiration and fluidic environment is crucial for personalized healthcare. However, wearable sensors based on triboelectric effect, piezoelectric effect, capactive (Huang, Y.-C., et al. Sensitive pressure sensors based on conductive microstructured air-gap gates and two-dimensional semiconductor transistors. Nat. Electron. 3, 59-69 (2020)), and transistor (Schwartz, G., et al. Flexible polymer transistors with high pressure sensitivity for application in electronic skin and health monitoring. Nat. Commun. 4, 1859 (2013)) technologies are vulnerable to ambient humidity, and encapsulation layers will compromise their accuracy and wearability. As a result, the ability to endure the ambient humidity is an essential property for a wearable sensor.

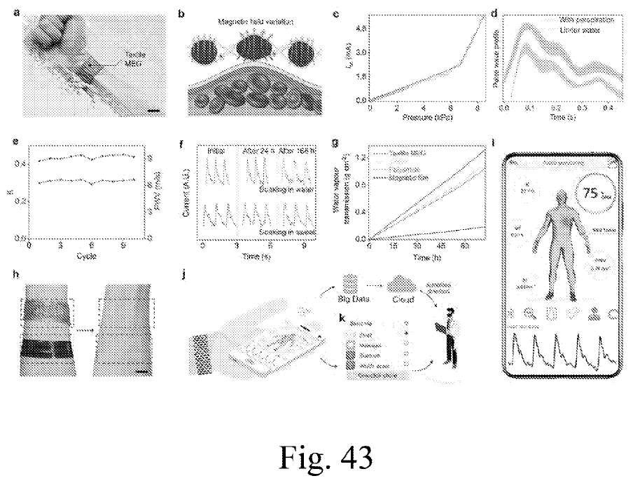

With a fundamentally new working principle, the textile MEG is a promising candidate in continuous arterial pulse monitoring for it can operate steady in humid environment without compromising its electrical performance and wearability. To demonstrate the textile MEG as an encapsulation-free and intrinsically waterproof pulse wave sensor for continuous cardiovascular parameters measurement, the textile MEG was fabricated into a wristband and worn against the wrist artery in an underwater situation (FIG. 43a). Pulsation of blood vessels induce soft magnetic microfiber deformation, which will sensitively cause the magnetic field variation within the soft microfiber due to the strong magnetomechanical coupling. As a consequence, the tiny arterial pressure fluctuations are converted into high-quality electrical signals via the textile wristband for further cardiovascular system characterization (FIG. 43b). To study the sensitivity of the textile MEG, loading-unloading tests were conducted at 1 Hz with increased applied pressure. As shown in FIG. 43c, the textile MEG can detect pressure as low as 0.05 kPa. The current responses linearly with the increased pressure in the range from 0.1 kPa to 6.52 kPa, and then the current increase quicker in the high-pressure range, which may due to the non-linearity of giant magnetoelastic effect in the high-pressure range. Moreover, textile MEG also responses linearly with respect to different movement frequency (FIG. 48a).

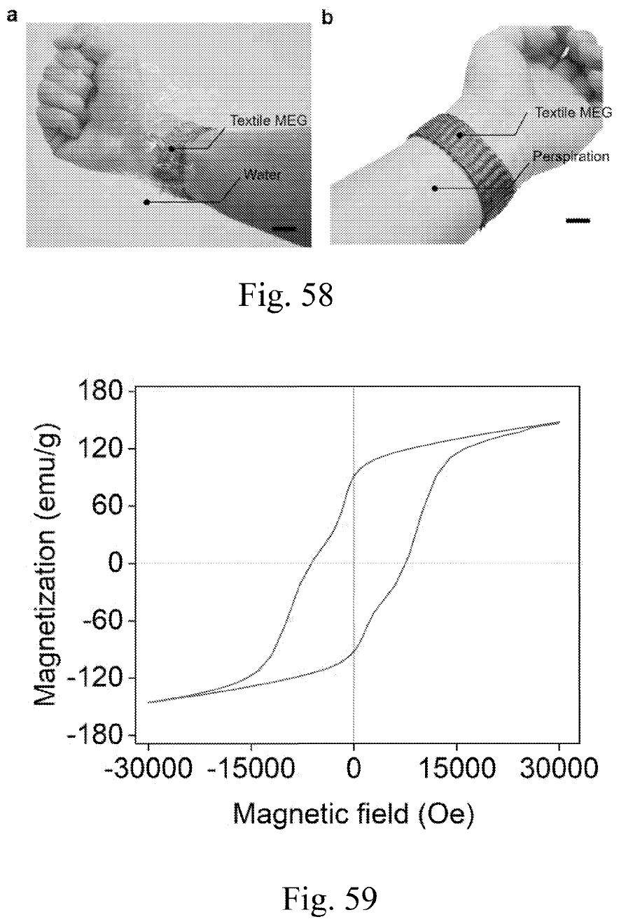

The generated pulse waveforms via arterial pressure fluctuations were collected by textile wristband and presented in in FIG. 44d, in which the shadow areas indicate the standard deviation. Three characteristic points of the arterial pulse wave including systolic, reflected and diastolic peaks are accurately expressed by the electrical signals no matter tested with perspiration or under water (FIG. 58). Embodiments took a further step to perform the cardiovascular parameters analysis. Heart rate (HR) are derived from peak position extracted from detected pulse waveform. Characteristic K values indicates the mean arterial blood pressure, and pulse wave velocity (PWV) reflects the elasticity and compliance of artery, which were plotted in the FIG. 43e. The stiff index (SI) is sensitive to the artery stiffness, and upstroke time (UT) represents the ascending time of the systolic stage (Chen, S., et al. Hierarchical elastomer tuned self-powered pressure sensor for wearable multifunctional cardiovascular electronics. Nano Energy 70, 104460 (2020)), which were also presented in the FIG. 48b. These five characteristic parameters HR, K, PWV, SI, and UT, are highly correlated with in pathological changes. Detailed calculations are presented in in Supplementary Note 2 below. Furthermore, both the mechanical stability against deformation and chemical stability against sweat corrosion are of great importance to the textile wristband for long-term continuous biomonitoring. On one hand, loading and unloading mechanical deformation up to 20,000 times was applied to the textile wristband. No obvious decay was observed from the electrical output (FIG. 48c). On the other hand, artificial perspiration was employed to test its chemical stability. As the results presented in FIG. 43f, the encapsulation-free textile wristband still can accurately detect the pulse waveforms after being immersed in water or artificial perspiration for up to 168 hours. These experimental observations fully demonstrated that the textile MEG is corrosion resistant and durable, owing to its stable remnant magnetization and large coercive field of nanomagnets (FIG. 59) (Cui, J., et al. Nanomagnetic encoding of shape-morphing micromachines. Nature 575, 164-168 (2019)).

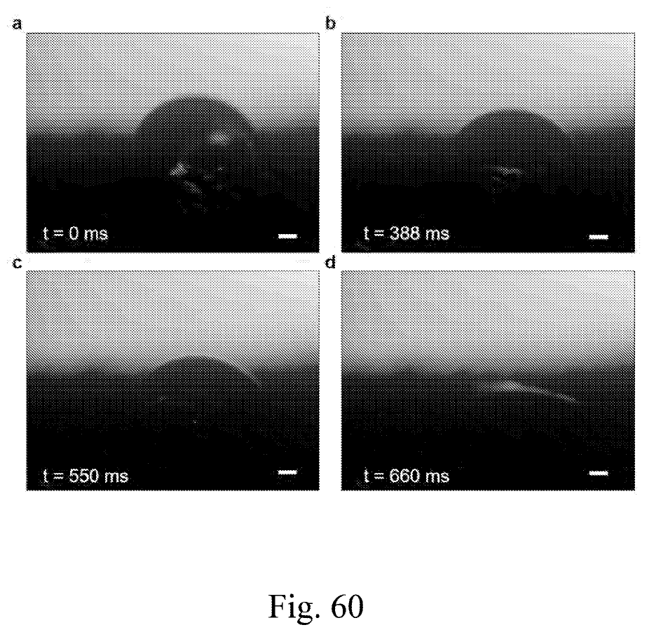

Furthermore, the wearability of the textile MEG is also an important factor for long-term cardiovascular monitoring, so embodiments further tested its permeability. As shown in FIG. 43g, the textile MEG has highest water vapor transmission rate of 0.018 g cm⁻² h⁻¹, which is higher than cotton, polyamide film, and magnetic film. This is due to the porous structure design of the textile and nanoscale-to-microscale cavities in the soft magnetic microfibers. Then, an on-skin test was done to prove its wearability. As shown in FIG. 43h, textile MEG did not cause negative effect on the skin after wearing for one week. However, the impermeable magnetic film caused skin irritation. This is mainly due to the perspiration can be absorbed quickly by textile MEG (FIG. 60) and water vapor can easily escape to the ambient environment, which ensures the wearer's comfort.

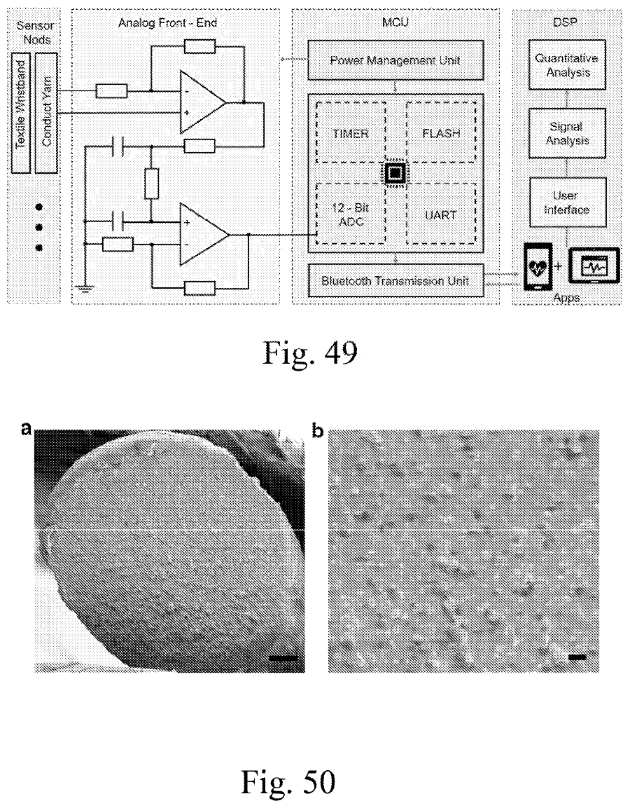

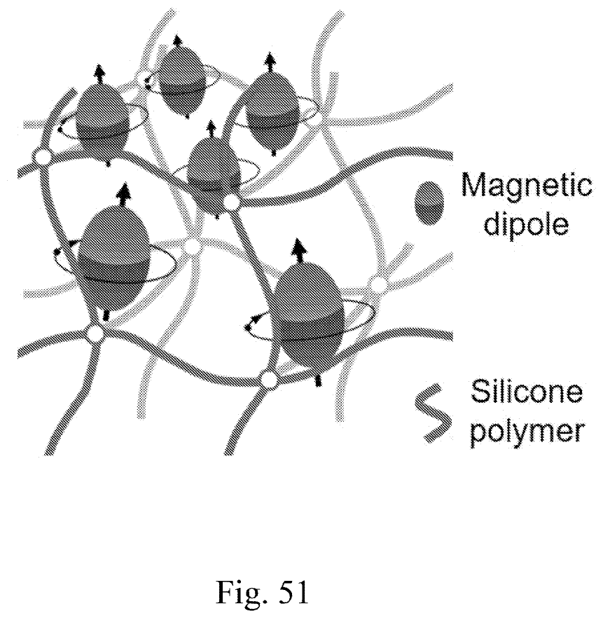

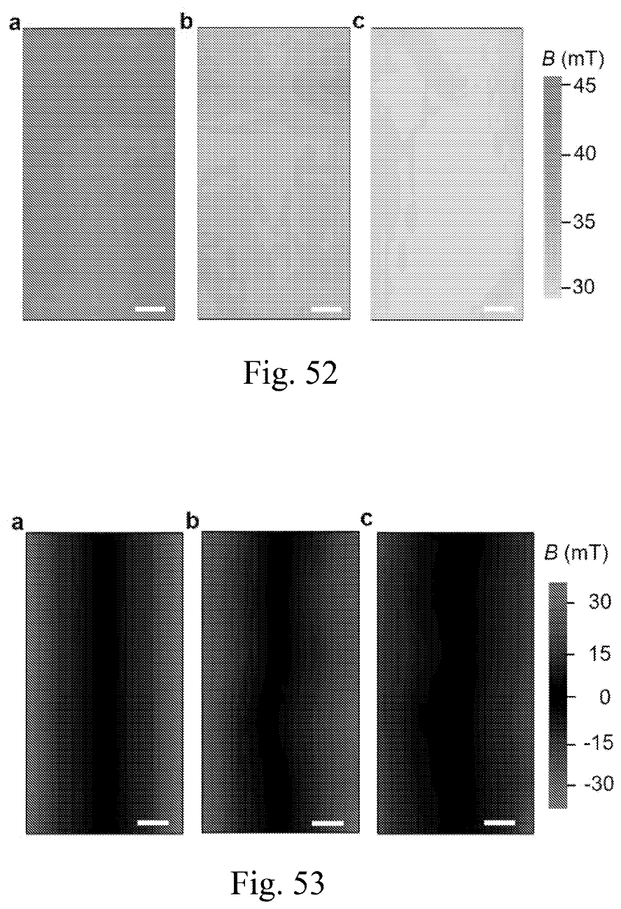

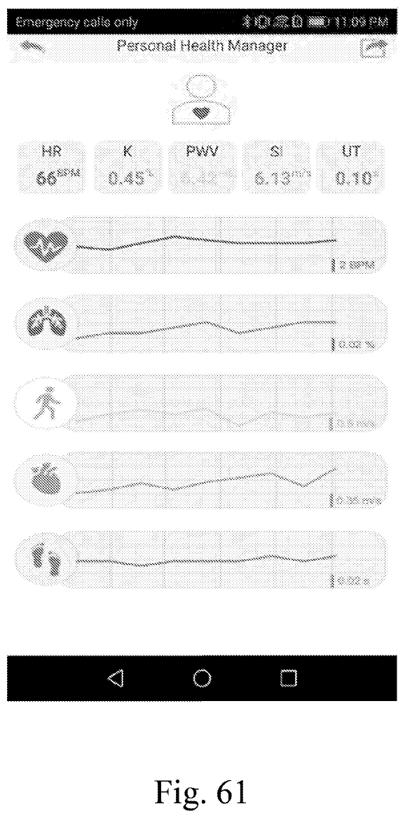

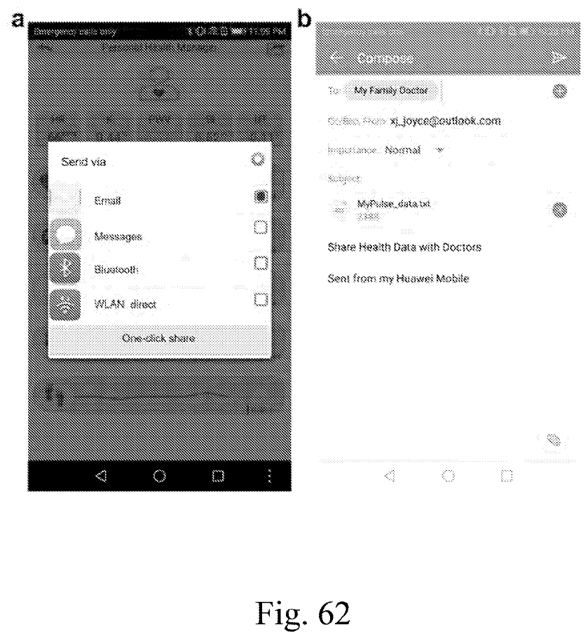

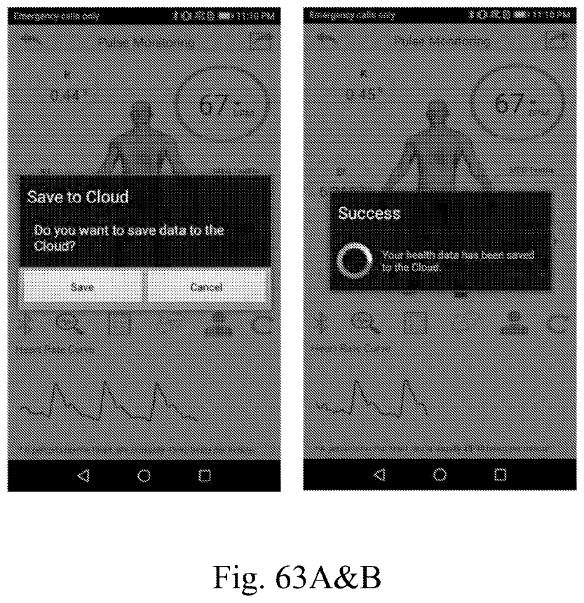

For user-friendliness toward practical application, a customized mobile APP with built-in algorithm was developed and integrated into the textile wristband to form a wireless cardiovascular monitoring system (CMS) for continuous measurement. As the system-level block diagram illustrated in FIG. 49, the CMS begins with pulse signal acquisition via the textile wristband; after signal conditioning and data processing, pules wave signals were wirelessly transmitted to cellphone with a Bluetooth module. With the built-in algorithm, the wearable CMS is able to analyze the real-time pulse wave signals, measure the characteristic parameters including HR, K, PWV, SI, and UT, and timely display the health data on the cellphone App (FIG. 43i). In order to present a comprehensive evaluation of individual cardiovascular system, five sets of curves were continuously displayed to track the dynamic change of characteristic parameters HR, K, PWV, SI, and UT, as the screenshot of the cellphone App demonstrated in FIG. 61. As illustrated in FIG. 43j, in the designed wireless CMS, the generated personal heath data could be further processed via two pathways. One is directly sent to the physicians for immediate clinical diagnosis. Through a simple one-click, the datasets could be sent to physicians via email, message, Bluetooth, or wireless local area network (WLAN) direct, as the screenshots of the App shown in FIG. 43k and FIG. 62. The other pathway is to upload the measured health data into the cloud to build up personal health database for big data analysis, which is available to the physician for data-driven diagnosis via authorized downloading, as the screenshot shown in FIG. 63. In order to visualize the operation features of the developed wireless CMS, a movie was recorded to demonstrate the wearable cardiovascular system management in the heavily sweaty situation. In summary, the intrinsically waterproof textile MEG enabled wireless CMS is providing a comfort and comprehensive solution for continuous cardiovascular system management. It represents a practical step to build up soft-matter technology towards telemedicine for personalized healthcare in the era of Internet of things.

### B. Discussion

The giant magnetoelastic effect can convert the tiny pressure to enormous magnetic field variation via altering the magnetic dipole interaction in a microfiber. To explore the strong magnetomechanical coupling in the soft system, an origami-programmed textile MEG was developed for high-performance biomechanics induced electricity generation. Compared to other existing textile counterparts, the origami-programmed textile MEG distinguishes itself in many aspects, including intrinsic humidity-resistance, high current, and low internal resistance.

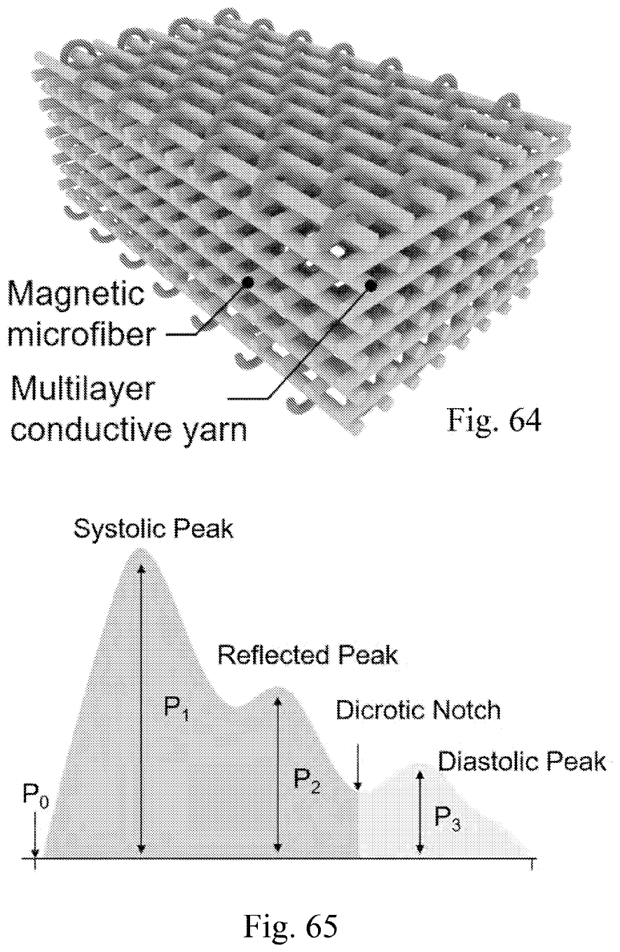

From fundamental science perspective, the origami-programmed textile MEG is built on a two-step conversion mechanism coupling giant magnetoelastic effect and magnetic induction. The discovered giant magnetoelastic effect in the soft fiber assures the highly efficient mechanical-to-magnetic conversion. It distinguishes from magnetoelastic metal alloys in larger magnetomechanical coefficient, lower Young's modulus, and no required magnetic field, as summarized in Supplementary Table B-1. With a following step of magnetic-to-electrical conversion, the origami-programmed textile MEG exhibits an ultrahigh short-circuit current density of 0.63 mA cm⁻² and ultralow internal impedance of 180Ω, corresponding to three orders of magnitude improvement than other textile counterparts based on triboelectric effect and piezoelectric effect, as presented in the Supplementary Table B-2 for a comprehensive comparison. This is due to their different working mechanisms. The present textile MEG is mainly based on mechanical force induced magnetic dipole realignment in the soft microfibers, whereas triboelectricity and piezoelectricity based textiles arise from their capacitive power generation principle via electric dipole alignment. Technically, the power density of the textile MEG can be further improved by weaving multilayer conductive yarns and soft magnetic microfibers into a 3D form, as illustrated in FIG. 64. It is envisioned that textile MEGs with versatile configurations represent a novel form of soft-matter electronic for energy, medical care, miniaturized robotics, and artificial perception.

The newly discovered giant magnetoelastic effect in soft microfibers is not restricted to being coupled with magnetic induction for electricity generation as demonstrated here. It is foreseen that it could be widely adopted to establish various soft-matter technologies and open doors to many fields. For instance, when it is coupled with magnetocaloric effect, the thermodynamics of a materials could be controlled with applied stress to present a mechanical force driven active personal thermoregulation. When it is coupled with magneto-optic effects, the refractive index of a materials could be altered in response to applied stress to invent various soft matter systems for mechanical force induced optical regulation for controlling the transmission, reflection, and absorption of light.

### B. Conclusions

In summary, the present Applicant discovered the giant magnetoelastic effect via magnetic dipole-dipole interaction in soft microfibers that shows up to 8.4 times greater magnetomechanical coupling than traditional magnetoelastic effect in metal alloys. Based on the microfibers, an origami-programmed textile MEG is constructed as an emerging approach for intrinsically waterproof wearable biomechanical energy conversion. With heavy perspiration, the textile MEG was demonstrated to deliver an unprecedented short-circuit current density of 0.63 mA cm⁻² with a low internal impedance of 180Ω. A textile wristband was also developed to convert the arterial pulse wave into high-quality electrical signals in an underwater scenario. A customized mobile APP with built-in algorithm was integrated into the textile wristband to form a wireless CMS, which could provide telemedicine via both immediate clinical and data-driven diagnosis. With features like intrinsic humidity-resistance, high current, and low internal resistance, envisioned are textile MEGs with versatile forms that can be widely adopted to build up versatile soft matter electronics. Moreover, it is worth noting that the giant magnetoelastic effect in 1D soft microfibers could also be further coupled with magneto-optic effect and magnetocaloric effect to invent compelling soft-matter technologies and open doors to pressure induced thermal and optical regulations.

### B. Example Methods

Fabrication of 1D soft magnetitic microfiber. The neodymium-iron-boron (NdFeB) nanomagnets (Magnequench) were firstly mixed with Ecoflex 00-30 part A (Smooth-on Inc.). Then, the Ecoflex 00-30 part B (Smooth-on Inc.) was added to the mixture with the same weight as part A. The weight percent of the NdFeB nanomagnets varies from 50 wt %, 75 wt %, and 83 wt % with the silicone polymer. After mixing thoroughly for 10 minutes to introduce microbubbles, the three-phase mixture was added to an extruding machine and pulled out via a nozzle to obtain the soft magnetic microfiber with tunable diameters. After heating the fibers for 30 minutes at 60° C., the soft magnetic microfiber was magnetized at impulse fields (2.65T) by an impulse magnetizer (IM-10-30, ASC Scientific) with programmed directions. Soft magnetic microfibers made by 83 wt % of SrFe₁₂O₉ (Sigma Aldrich) or Fe₃O₄ (Sigma Aldrich) are also fabricated with the same procedure as NdFeB.

Fabrication of a conductive yarn. A layer of silver was deposited on the nylon microfibers (Systcom Advanced Materials Inc.) by a chemical plating method. Then, the silver-coated nylon microfibers were intertwined by a multiaxial yarn winding machine to a conductive yarn.

Textile fabrication by a weaving loom. The textile MEG was fabricated by an industrial weaving loom via a shuttle flying process. The stress intension of the string was kept constant in the weaving process by a specially designed pully system. For the vertical weaving pattern, the soft magnetic microfibers were employed as the fixed shuttle, the conductive yarns were inserted as the flying shuttle. To obtain the parallel weaving pattern, nylon wires were employed as the fixed shuttle, the conductive yarn and soft magnetic microfibers were inserted as the flying shuttle. The detailed description of the weaving process is illustrated in Supplementary Note 3. For the textile MEG used in CMS, silver-coated yarns were employed as conductive yarn, and the textile MEG is fabricated with size of 3 cm by 13 cm. For the biomechanical energy harvesting application, copper wires were employed as conductive yarn. To charge capacitors, the textile MEG is fabricated with size of 4 cm by 6 cm using conductive yarns and soft magnetic microfibers with cross section area of 12 mm². Moreover, wool fibers were also woven into the textile to improve the textile wearability. It is worth noting that both the fibers fabrication and textile weaving process are straightforward and compatible with massive production.

Water vapor transmission rate test. The test was based on ASTM E94 with modification. Glass bottles were filled with deionized water and sealed by tested textile samples. The glass bottles were placed in an environment of 35° C. and were weighted by an electronic balance. By calculated the reduced mass of the bottles divided by area of textiles, the water vapor transmission rate was obtained.

Preparation of artificial perspiration. The artificial perspiration was used to test the chemical stability of the textile wristband. To prepare it, 4.65 g NaCl (Sigma Aldrich), 3.87 g 1 M lactic acid solution (Alfa Aesar), 1.80 g Urea (Alfa Aesar), 1.37 g KCl (Sigma Aldrich), 0.756 g NaHCO₃(Sigma Aldrich), 0.546 g 1 M NH₃·H₂O (Sigma Aldrich), 0.175 g Na₂SO₄ (Sigma Aldrich), and 0.0276 g uric acid (Alfa Aesar) were added in 3 L deionized water and mixed for 30 minutes.

Structure characterization. The morphology of the soft magnetic microfiber and conductive yarn were characterized by scanning electron microscopy (Zeiss, supra 40VP). The micro computed tomography (Micro CT) image of the magnetic microfiber was characterized by Micro CT (CrumpCAT). The 2D magnetic flux density mapping on the surface of the soft magnetic microfiber was achieved by continuous measuring the magnetic field using the digital Gauss meter mounted on a three axial motion platform. The stress-strain curves were tested at a stretching rate of 0.25 mm s⁻¹. The Young's modulus was calculated via fitting the tested curves with a Neo-Hookean model. Magnetic hysteresis loop was tested by a SQUID magnetometer (MPMS3, Quantum Design).

Electrical performance measurement. The voltage and current signals of the textile MEG were measured by electrometer (6514, Keithley). A flat plate larger than the size of the textile were used to replace human hands for standard test. The flat plate was connected with an electrodynamic shaker system, which consists of a function generator (AFG1062, Newark), a linear power amplifier (PA-151, Labworks Inc.) and an electrodynamic transducer (ET-126HF, Labworks Inc.). To charge capacitors, the generated electricity was processed with a diode bridge rectifier (MBSK16SE) and a toroidal transformer.

Design of the circuitry and user interface. A customized printed circuit board (PCB) was designed to acquire the pules wave signals from textile wristband. The PCB has three components. Firstly, an analog pulse signal acquisition is used for signal conditioning. Next, a micro-controller unit (MCU, STM32) is used to collect and convert the analog pulse signals to digital pulse signal, and thirdly a Bluetooth module (HC-05, XM electronic) is used to wirelessly transmit the digital data to cellphone. The cellphone APP was created via MIT AI2 Companion. The heart rate, pulse wave velocity, stiffness index, K value and upstroke time were calculated with the assistance of the peak detection algorithms.

### B. Supplementary Note 1: Theoretical Explanation of the Wavy Chain Analytical Model

The giant magnetoelastic effect in soft magnetic microfibers can be described using wavy chain model with inner dipole-dipole interaction. It is assumed that under impulse magnetization, the nanomagnets, which can be regarded as single magnetic dipoles (Borbáth, T., Günther, S., Yu Borin, D., Gundermann, T. & Odenbach, S. XμCT analysis of magnetic field-induced phase transitions in magnetorheological elastomers. Smart Mater, Struct. 21, 105018 (2012)), align in a zig-zag wavy chain structure as illustrated in FIG. 41i. Further assumed is that dipole-dipole interaction exist only inside the wavy chain according to previous literature (Han, Y., Hong, W. & Faidley, L. E. Field-stiffening effect of magneto-rheological elastomers. Int. J. Solids Struct. 50, 2281-2288 (2013)). To simplify the calculation, each magnetic dipole is considered to have a vertical magnetic dipole moment m which is reasonable owing to the system symmetry. Based on the above assumption, the magnetic interaction energy per dipole inside the chain Uᵢₙₜ can be expressed as,

$${U}_{int} = \frac{{μ}_{0} m \text{?}}{π {λ}^{2} h \text{?}} β ( \frac{l}{h λ \text{?}} ) \qquad ( 1 )$$

$$β ( \frac{l}{h λ \text{?}} ) = \frac{1}{2} {\sum }_{n = 1}^{∞} ( {( \frac{l}{h λ \text{?}} )}^{2} - 2 {( 2 n - 1 )}^{2} ) {( {( \frac{l}{h λ \text{?}} )}^{2} + {( 2 n - 1 )}^{2} )}^{- \text{?}} - 0.1503 \qquad ( 2 )$$

$$\text{?} \text{indicates text missing or illegible when filed}$$

where λ is the principal stretch in the compress direction, μ₀ is the vacuum permeability, h and l denote the vertical and horizontal distances between two adjacent magnetic dipoles in the wavy chain, respectively. β(x) is the dipole alignment factor describing the magnetic interaction energy that all other magnetic dipoles contribute to a single dipole in the wavy chain. It is worth noting that when l=0, the wavy chain reduces to a straight chain and

$$β ( \frac{l}{h λ \text{?}} )$$

$$\text{?} \text{indicates text missing or illegible when filed}$$

degenerates to −1.2.

The dipole moment m can be further written in a weak magnetic form approximation as,

$$m = 4 π {r}^{3} \frac{{B}_{r}}{{μ}_{0}} \qquad ( 3 )$$

where Bᵣ is the approximate remnant magnetic flux density and r is radius of the magnetic dipole. Apart from the interaction energy, each magnetic dipole also has magnetic energy which can be described below (Diguet, G, Sebald, G., Nakano, M., Lallart, M. & Cavaillé, J.-Y. Magnetic particle chains embedded in elastic polymer matrix under pure transverse shear and energy conversion. J. Magn, Magn. Mater. 481, 39-49 (2019));

$${U}_{d} = \frac{1}{2} \frac{{B}_{r}^{2}}{{μ}_{0}} \frac{4}{3} π {r}^{3} \qquad ( 3 )$$

Combining magnetic potential energy from both dipole-dipole interaction and magnetic dipoles, the total magnetic potential energy can be calculated as,

$$U = {U}_{d} + {U}_{int} \qquad ( 4 )$$

Considering the average magnetic dipole density inside the soft magnetic fiber is N, Then the magnetic potential energy in a small portion of the microfiber could be expressed as

$$W = NU \qquad ( 5 )$$

Combining equation 1 to equation 5, the magnetic potential energy per small volume is

$$W = \frac{\text{?}}{\text{?}} \frac{\text{?}}{\text{?}} \frac{\text{?}}{\text{?}} \text{?} + \frac{\text{?}}{\text{?}} \frac{\text{?}}{\text{?}} β ( \frac{\text{?}}{\text{?}} ) \qquad ( 6 )$$

$$\text{?} \text{indicates text missing or illegible when filed}$$

From the magnetic potential energy, the magnetic field can be approximated accordingly, using the following equation,

$$H = \frac{\text{?}}{\text{?}} = N \frac{\text{?}}{\text{?}} \frac{\text{?}}{\text{?}} \text{?} + \frac{\text{?}}{\text{?}} \frac{\text{?}}{\text{?}} β ( \frac{\text{?}}{\text{?}} ) \qquad ( 7 )$$

$$\text{?} \text{indicates text missing or illegible when filed}$$

From equation 7, the magnetic field is related to the principal stretch λ of the magnetic microfibers. λ=1 represents the initial state and λ<1 represents the magnetic field under compressive mechanical stress. As a result, the ratio of magnetic field under compressive stress H₁ to initial magnetic field H₀ is given as below,

$${H}_{1} / {H}_{0} = ( 1 + 24 β ( \frac{1}{\text{?}} ) \text{?} {( \frac{\text{?}}{\text{?}} )}^{\text{?}} ) / ( 1 + 24 β ( \frac{\text{?}}{\text{?}} ) \text{?} {( \frac{\text{?}}{\text{?}} )}^{\text{?}} ) \qquad ( 8 )$$

$$\text{?} \text{indicates text missing or illegible when filed}$$

With measured values of r=2.5 μm, h=7.8 μm, l=9.36 μm and G=630 kPa for the soft magnetic microfiber, and the compressive stress s through an incompressible Neo Hookean model below

$$\text{?} = G ( λ - 1 / {λ}^{2} ) \qquad ( 9 )$$

$$\text{?} \text{indicates text missing or illegible when filed}$$

the wavy chain model accurately captures its magnetic field variation in response to the compressive stress changing from 0 to 450 kPa, which is well consistent with the experimental observation in FIG. 40g. It is worth noting that the derived H₀/H₁ only approximately represent the ideal case in which the edging effect and the shape of magnetic fiber were not considered.

### B. Supplementary Note 2: Cardiovascular Parameters Analysis

The circulation of blood will transmit oxygen and nutrient within body. Heart rate is an essential parameter during this process. Heart rate can be detected when heart pumps the blood, which generates pulse waves. Using textile MEG to percept tiny pressure fluctuations of the blood vessel, high-quality electrical signals can be obtained. After calculating the average heartbeat in one minute, one can obtain the heart rate. A normal heart rate is usually between 45-90 beats per minute

As shown in Supplementary FIG. 65, in one single pulse, there are three peaks, i.e., systolic peak (P1), reflected peak (P2), and diastolic peak (P₃). In this work, embodiments characterize K values, pulse wave velocity (PWV), stiff index (SI) and upstroke time (UT) by analyzing the parameters in the pulse wave profile. K values indicates the mean arterial blood pressure, which can be calculated by the following two equations,

$$\text{?} = \text{?} {\int }_{\text{?}}^{\text{?}} \text{?} ( t ) dt \qquad ( 1 )$$

$$K = \frac{\text{?} - \text{?}}{\text{?} - \text{?}} \qquad ( 2 )$$

$$\text{?} \text{indicates text missing or illegible when filed}$$

where Pₘ is the integration of the peak. P₀ and P₁ can be acquired from the pulse wave profile shown in FIG. 65. For healthy adults, the K value is normally less than 0.45.

PWV reflects the elasticity and compliance of the artery, which can reflect the degree of arterial stiffness. Larger PWV value indicates possibility of severe stiffness. The PWV values are calculated by using the following equation,

$$PWV = 0.8 \times \frac{2 Δ L}{\text{?} W \text{?}} \qquad ( 3 )$$

$$\text{?} \text{indicates text missing or illegible when filed}$$

ΔL is the distance from jugulum to pubic symphysis, whereas RWTT is the time between systolic peak and reflected peak. The normal distribution of the PWV values is between 3.8 to 9.2 m s⁻¹, and the mean PWV value is 6.5 m s⁻¹.

The SI is sensitive to the artery stiffness and was calculated based on the following equation,

$$\text{?} - \frac{\text{?}}{\text{?}} \qquad ( 4 )$$

$$\text{?} \text{indicates text missing or illegible when filed}$$

H is the height of the subject, whereas PPT is the time between P₁ and P₃. In general, the SI value is less than 10 m s⁻¹. UT represents the ascending time of the systolic stage, which can be obtained by calculating the time interval between the P₀ and P₁.

### B. Supplementary Note 3: The Weaving Process Via a Shuttle-Flying Process

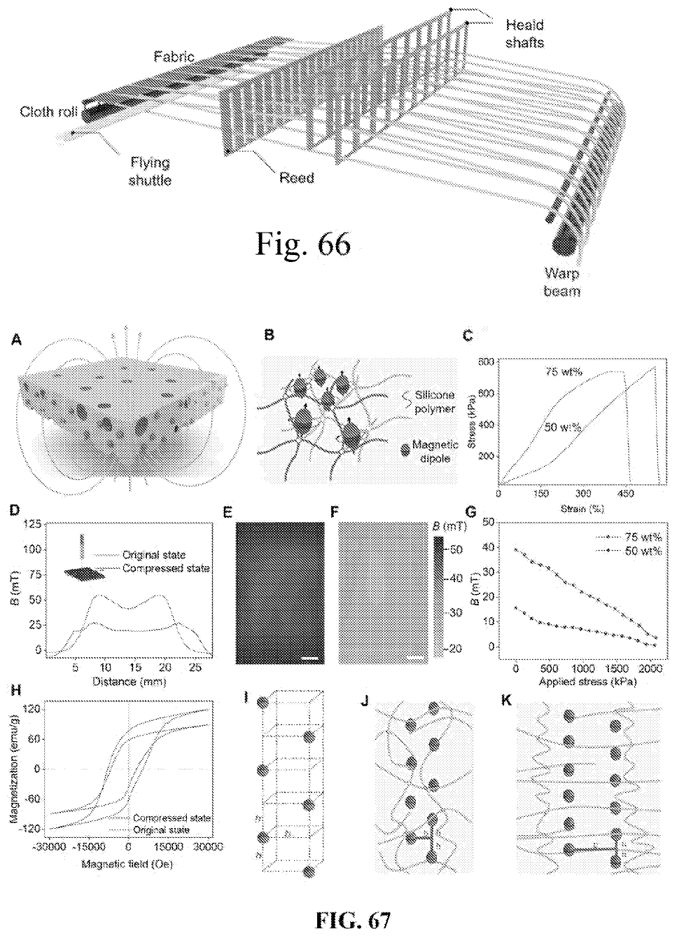

The weaving loom is used to integrate the conductive yarns and the soft magnetic microfibers together. There are five parts in a typical weaving loom, including cloth roll, flying shuttle, reed, heald shafts, and warp beam (FIG. 66). For the vertical weaving pattern, the soft magnetic microfibers were employed as the fixed shuttle in the longitudinal direction. After all magnetic microfibers were rolled in the warp beam, they were inserted in the heald shafts, and were fastened in the cloth roll. The tensional stress on the soft magnetic microfibers was calibrated by a gauge meter to make sure the stress on different soft magnetic microfiber remains the same. The conductive yarns were acted as the flying shuttle in the latitudinal direction. The shed is created by raising the warp yarns using two heald shafts. Every time when the flying shuttle (filling conductive yarns) is inserted in the shed, two heald shafts will exchange their positions by turning the compound roller. After the weaving procedure, the conductive yarns were connected in series.

For the parallel pattern, nylon wires were employed as the fixed shuttle in the longitudinal direction. After all nylon wires were rolled in the warp beam, they were inserted in the heald shafts, and were fastened in the cloth roll. The tensional stress of the nylon wires was also calibrated with the method same as the soft magnetic microfibers. The conductive yarn and soft magnetic microfibers were inserted as the flying shuttles in the latitudinal direction. The weaving method is the same as the vertical weaving pattern. Conductive yarns and the magnetic microfibers were weaved alternatively into the fabric. After the weaving procedure, the conductive yarns on two sides were connected in series. It is worth noting that wool fibers can also be woven in the textile to improve the textile wearability. Additionally, both the fibers and the fabric weaving process are compatible with mass production (Qin, Y., Wang, X. & Wang, Z. L. Microfibre-nanowire hybrid structure for energy scavenging. Nature 451, 809-813 (2008): Zeng, W., et al. Highly durable all-fiber nanogenerator for mechanical energy harvesting. Energy Environ. Sci. 6, 2631-2638 (2013); Guan, X., Xu, B. & Gong, J. Hierarchically architected polydopamine modified BaTiO3@P (VDF-TrFE) nanocomposite fiber mats for flexible piezoelectric nanogenerators and self-powered sensors. Nano Energy 70, 104516 (2020); Anwar, S., et al. Piezoelectric nylon-11 fibers for electronic textiles, energy harvesting and sensing. Adv. Funct. Mater. 31, 2004326 (2020); Pu, X., et al. A self-charging power unit by integration of a textile triboelectric nanogenerator and a flexible lithium-ion battery for wearable electronics. Adv. Mater. 27, 2472-2478 (2015); Chen, J., et al. Micro-cable structured textile for simultaneously harvesting solar and mechanical energy. Nat. Energy 1, 16138 (2016)).

### C. Self-Powered Biomechanical Sensor Using Giant Magnetoelastic Effect in Soft-Matter Electronics

### C. Summary

According to a third aspect, the present embodiments relate to a giant magnetoelastic effect in a soft system due to dipole-dipole interactions, which exhibit a 3.3 times larger magnetomechanical coefficient than what is found in the most commonly used alloys. To investigate the mechanism of the giant magnetoelastic effect, embodiments established a wavy chain analytical model based on magnetic dipole-dipole interactions. In order to demonstrate the feasibility of giant magnetoelastic effect in soft-matter electronics, embodiments further coupled the giant magnetoelastic effect with magnetic induction to make a self-powered biomechanical sensor with stretchability up to 550%. By manipulating dipole alignment, it has achieved an extremely wide sensing range from 3.5 Pa to 2,000 kPa (˜20 times larger than that of sensors in other categories) with a response time ˜5 ms. The magnetoelastic pressure sensor was demonstrated for both wearable physiological monitoring and implantable heart activities sensing. With a collection of compelling features including minimal hysteresis, ultra-wide sensing range, waterproofness, and biocompatibility, the magnetoelastic sensor paves a compelling new way for self-powered biomechanical sensing.

### C. Introduction

Magnetoactive soft materials, such as magnetorheological elastomers and magnetic gels, have been widely investigated in recent years due to their properties of magnetostriction (J. M. Ginder, S. M. Clark, W. F. Schlotter, M. E. Nichols, Magnetostrictive phenomena in magnetorheological elastomers. Int. J. Mod. Phys. B 16, 2412-2418 (2002)) and tunable structural geometry upon an external magnetic field (R. Zhao, Y. Kim, S. A. Chester, P. Sharma, X. Zhao, Mechanics of hard-magnetic soft materials. J. Mech. 306 Phys. Solids 124, 244-263 (2019)). Consequently, they have been used as vibration absorbers (X. Guan, X. Dong, J. Ou, Magnetostrictive effect of magnetorheological elastomer. J. Magn. Magn. Mater. 320, 158=163 (2008); H. X. Deng, X. L. Gong, Adaptive tuned vibration absorber based on magnetorheological elastomer. J. Intell. Mater. Syst. Struct. 18, 1205-1210 (2016)), magnetoresistive sensors (I. Bica, E. M. Anitas, M. Bunoiu, B. Vatzulik, I. Juganaru, Hybrid magnetorheological elastomer: Influence of magnetic field and compression pressure on its electrical conductivity. J. Ind. Eng. Chem. 20, 3994-3999 (2014)), and soft robots (Y, Kim, H. Yuk, R. Zhao, S. A. Chester, X. Zhao, Printing ferromagnetic domains for untethered fast-transforming soft materials. Nature 558, 274-279 (2018); J. Cui et al., Nanomagnetic encoding of shape-morphing micromachines. Nature 575, 164-168 (2019)) (Note S1). The inverse effect of magnetostriction, however, has been previously ignored in these soft systems. The magnetoelastic effect, namely, the change of the magnetic property in a material upon mechanical deformation, is usually observed in metal alloys, such as Fe₁₋ₓCoₓ (S.-i. Yamaura, T. Nakajima, T. Satoh, T. Ebata, Y. Furuya, Magnetostriction of heavily deformed Fe—Co binary alloys prepared by forging and cold rolling. Mater. Sci. Eng. B 193, 121-129 (2015)), TbₓDy₁₋ₓFe₂ (Terfenol-D) (Q. Su, J. Morillo, Y. Wen, M. Wuttig, Young's modulus of amorphous Terfenol-D thin films. J. Appl. Phys. 80, 3604-3606 (1996)), and GaₓFe₁₋ₓ (Galfenol) (S. Datta, J. Atulasimha, C. Mudivarthi, A. B. Flatau, Stress and magnetic field-dependent Young's Modulus in single crystal iron-gallium alloys. J. Magn. Magn. Mater. 2135-2144 (2010)), which are applied in building vibration monitoring (G. Ausanio et al., Magnetoelastic sensor application in civil buildings monitoring. Sens. Actuator A Phys. 123-124, 290-295 (2005) and magnetization control for cell sorting (R. Khojah et al., Single-domain multiferroic array-addressable Terfenol-D (SMArT) micromagnets for programmable single-cell capture and release. Adv. Mater. 33, 2006651 (2021)). However, this effect has been excluded from soft systems for two reasons. First, to observe a decent conversion efficiency, usually, megapascals of mechanical stress are required to deform the metal alloys under an external magnetic field, leading to a complicated external electromagnetic system. Secondly, Young's modulus of metal alloys is usually in the order of tens of gigapascal, which is six orders of magnitude larger than that of the human body's mechanical modules, which presents an obstacle in the application of such effect in soft-matter electronics.

As described above, the present Applicant discovered a giant magnetoelastic effect in a soft system, based on the working principle of dipole-dipole interactions, which exhibit a 3.3 times larger magnetomechanical coefficient than that found in bulky alloys. Comparing to the previously reported magnetoelastic effect in metal alloys, the discovered giant magnetoelastic effect shows three advantages. First, in the developed soft polymer matrix, the external magnetic field is not required, which greatly simplifies the structure. Secondly, the Young's modulus of the soft system is compatible with the human body (C. Pailler-Mattei, S. Bec, H. Zahouani, In vivo measurements of the elastic mechanical properties of human skin by indentation tests. Med. Eng. Phys. 30, 599-606 (2008)). Thirdly, the required mechanical pressure is within the physiological range in the human body. Furthermore, to show its potential in soft-matter electronics, the present Applicant developed a wearable, ultra-stretchable magnetoelastic sensor by embedding super-elastic liquid metal microfibers into the magnetorheological elastomers. This effect was coupled with the giant magnetoelastic effect due to magnetic induction. The mentioned two-step conversion enables the ultra-stretchable magnetoelastic sensor to develop several advantages over other pressure sensors that are based on the triboelectric (X. Pu et al., A self-charging power unit by integration of a textile triboelectric nanogenerator and a flexible lithium-ion battery for wearable electronics. Adv. Mater. 27, 2472-2478 (2015); J. Chen et al., Micro-cable structured textile for simultaneously harvesting solar and mechanical energy. Nat. Energy 1, 16138 (2016)), piezoelectric (S. K Ghosh, D. Mandal, Synergistically enhanced piezoelectric output in highly aligned 1D polymer nanofibers integrated all-fiber nanogenerator for wearable nano-tactile sensor. Nano Energy 53, 245-257 (2018); Y. Qin, X. Wang, Z. L. Wang, Microfibre-nanowire hybrid structure for energy scavenging. Nature 451, 809-813 (2008); S. Anwar et al., Piezoelectric nylon-11 fibers for electronic textiles, energy harvesting and sensing. Adv. Funct. Mater. 31, 2004326 (2020); X. Guan, B. Xu, J. Gong, Hierarchically architected polydopamine modified BaTiO₃@P (VDF-TrFE) nanocomposite fiber mats for flexible piezoelectric nanogenerators and self-powered sensors. Nano Energy 70, 104516 (2020)), piezoresistive (J. He et al., A Universal high accuracy wearable pulse monitoring system via high sensitivity and large linearity graphene pressure sensor. Nano Energy 59, 422-433 (2019); S. Chun et al., An artificial neural tactile sensing system, Nat. Electron. 4, 429-438 (2021)), and capacitive effect (C. M. Boutry et al., A stretchable and biodegradable strain and pressure sensor for orthopaedic application. Nat. Electron. 1, 314-321 (2018)). First, it has a wide sensitivity range from 3.5 Pa to 2,000 kPa, which could cover diverse biomechanical pressure sensing at different locations of the human body. Secondly, the magnetoelastic sensor is ultra-stretchable up to 550% with an ultrafast response time within 5 ms. Also, it is biocompatible, verified by in vitro culture of human fibroblasts, meaning it could be used for implantable devices during rehabilitation protocols. Importantly, since the magnetic field could penetrate the water molecules, this magnetoelastic sensor is fully waterproof without the need for an encapsulation layer. Thus, the developed magnetoelastic sensor was tested on ex vivo porcine heart for the diagnosis of heart diseases and on the human body for the monitoring of physiological signals.

### C. Results

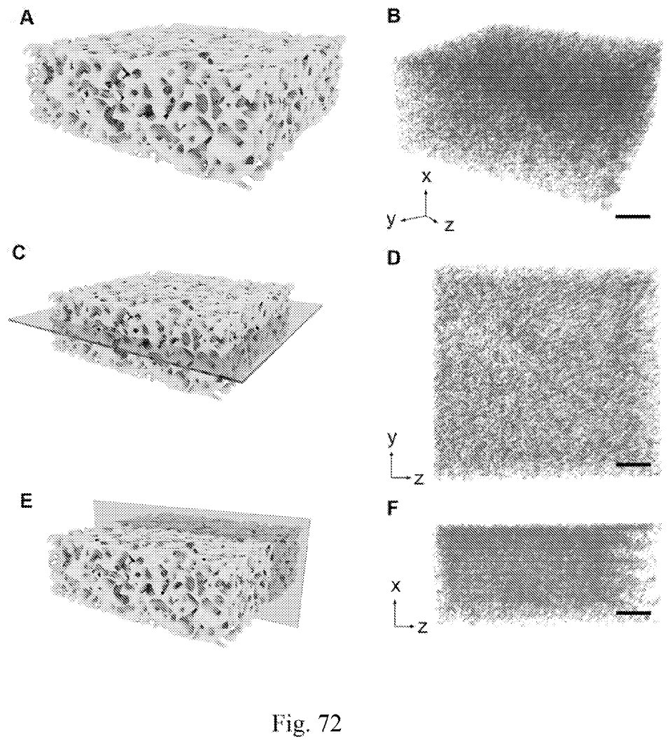

By mixing together a highly viscous silicone polymer, solid magnetic. nanoparticles, and air microbubbles, embodiments unraveled a giant magnetoelastic effect in a soft polymer composite (FIG. 67A). After that, the composite was magnetized with impulse fields that reorientated the nanomagnets in the soft polymer matrix with uniform polarity (FIG. 67B). Scanning electron microscopy (SEM) indicated that the nanomagnets were uniformly dispersed in the silicone polymer matrix (FIG. 71). The micro-computed tomography (Micro-CT) images also confirmed the uniformly dispersed nanomagnets (FIG. 72), and showed that there were microbubble cavities uniformly dispersed in the soft polymer composite, which are beneficial in decreasing the mechanical modulus and increasing the wearing comfort. In order to visualize its features, a dynamic 3D model was made as shown in Movie S1. The mechanical modulus and fracture strain could be controlled by different magnetic concentrations in the polymer matrix. As shown in FIG. 67C, the soft polymer composites with 50 wt % and 75 wt % magnetic concentration have Young's modulus of 182 kPa and 387 kPa, respectively.

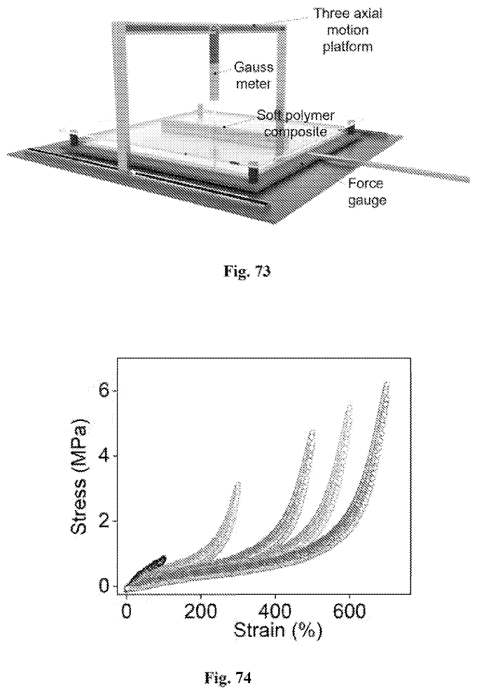

In order to investigate its magnetization variation upon mechanical stress, a customized three-axis platform was designed to illustrate magnetic variation under different uniaxial pressures (FIG. 73). The surface magnetic field distribution of the soft polymer composites is shown in FIG. 67D. Under uniaxial pressure of 1,000 kPa, the surface magnetic flux density along the line decreased dramatically. This observation is also justified by the magnetic flux density mapping on the south pole of the soft polymer composites as shown in FIGS. 67E and 67F. The magnetic flux density decreased on both the edge and middle of the soft polymer composite. Further plotted was the surface magnetic flux density under different uniaxial stresses. As exhibited in FIG. 67G, the magnetic flux density decreased with an increase in applied stress from 0 kPa to 2,080 kPa, and a maximum magnetomechanical coupling factor of 4.16×10⁻⁸ T Pa⁻¹ was derived from the curve, which was 3.3 times more enhanced than the Fe—Co alloy metal alloys (Table C-S1). Moreover, the magnetic hysteresis loops of the soft polymer composite in the initial and compressed states were tested by the superconducting quantum interference device technique (SQUID).

TABLE C-S1

| | | | Required | Magneto- | Applied |
|---|---|---|---|---|---|
| | | | external | mechanical | pressure |
| Material | Young's | | magnetic | coupling | range |
| category | modulus | | field (Oe) | factor (T/Pa) | (kPa) |
| Fe—Co alloy | 200 | GPa^{a} | 25^{c} | 1.25 × 10^{-8a} | 5000-70000 |
| Co | 209 | GPa^{a} | 10^{c} | 2.45 × 10^{-8a} | — |
| Terfenol-D | 19 | GPa^{a} | 950^{c} | 3.26 × 10^{-8a} | 11000-41000 |
| Galfenol | 54 | Gpa^{a} | 50^{c} | 2.13 × 10^{-8a} | 10000-50000 |
| Soft polymer | 629.76 | kPa^{b} | 0 | 4.16 × 10⁻⁸ | 0-2000 |
| matrix | | | | | |

^{a}Estimated value.
^{b}Soft polymer matrix with 75 wt % concentration.
^{c}Calculated based half of the saturation.

As shown in FIG. 67H, both the remnant magnetization and coercive field of the soft magnetic composite decreased under pressure. This might be due to the rearrangement of the nanomagnets inside the soft magnetic composites, caused by mechanical agitation. To investigate the mechanism of the giant magnetoelastic effect, a wavy chain model was established based on the magnetic dipole-dipole interaction and the demagnetizing factor. As illustrated in FIG. 67I, the nanomagnets form wavy chains in the polymer matrix under impulse magnetic fields.

When compressive stress is applied to the soft polymer composite, the corresponding shape deformation leads to the distance and orientation variation of the magnetic dipoles (FIG. 67J and FIG. 67K). As a result, the dipole-dipole interaction inside the wavy chain is associated with a decrease in the external magnetic field. Quantitatively, the magnetic field variation, in response to applied mechanical pressure, can be evaluated by H1⊥/H0⊥, which can be expressed as a function of the principle stretch λ, based on the wavy chain model as below.

$${H}_{1 ⊥} / {H}_{0 ⊥} \approx \frac{\frac{1}{( 2 a + 1 ) {λ}^{1.5 k}} + \frac{{r}^{3}}{3 {λ}^{3} {h}^{2}} ( 0.3006 - f ( \frac{l}{h {λ}^{1.5}} ) )}{\frac{1}{( 2 a + 1 )} + \frac{{r}^{3}}{3 {h}^{3}} ( 0.3006 - f ( \frac{1}{h} ) )} \qquad ( 1 )$$

where H1⊥ and H0⊥ represent the vertical magnetic field with and without applied mechanical pressure, respectively, r is the radius of the nanomagnet, a is the estimated aspect ratio of a single wavy chain structure.

In FIG. 67I, h, and l denote the vertical and horizontal distances between two adjacent magnetic dipoles in the wavy chain, respectively. 0.3006−f(x) is the dipole alignment factor describing the contribution of all other magnetic dipoles contributing to a single dipole in the wavy chain on the surface of the soft polymer composite. k represents a constant, characterizing the influence of the nonideality, neighboring chain-chain interaction, and macroscopic shape effect to the demagnetizing factor under compressive deformation. The derivation of the dipole alignment factor is detailed in Note S2. The magnetic field variation H1⊥/H0⊥ is further linked to the compressive stress s through an incompressible Neo-Hookean material model with the following equation,

$$s = G ( λ - 1 / {λ}^{2} ) \qquad ( 2 )$$

where G is the shear modulus of the soft magnetic composite. With an estimated value of a=105, r=2.5 μm, h=13.5 μm, and 1=14.85 μm in the soft magnetic composite, it was found that when k=0.05, the wavy chain model accurately captures its magnetic field variation in response to the compressive stress changing from 0 to 450 kPa.

With both the experimental results and theoretical verification, it is clear that only tiny external pressure could generate magnetic field variation in the soft polymer composite.

### C. Constructing an Example Ultra-Stretchable Magnetoelastic Sensor

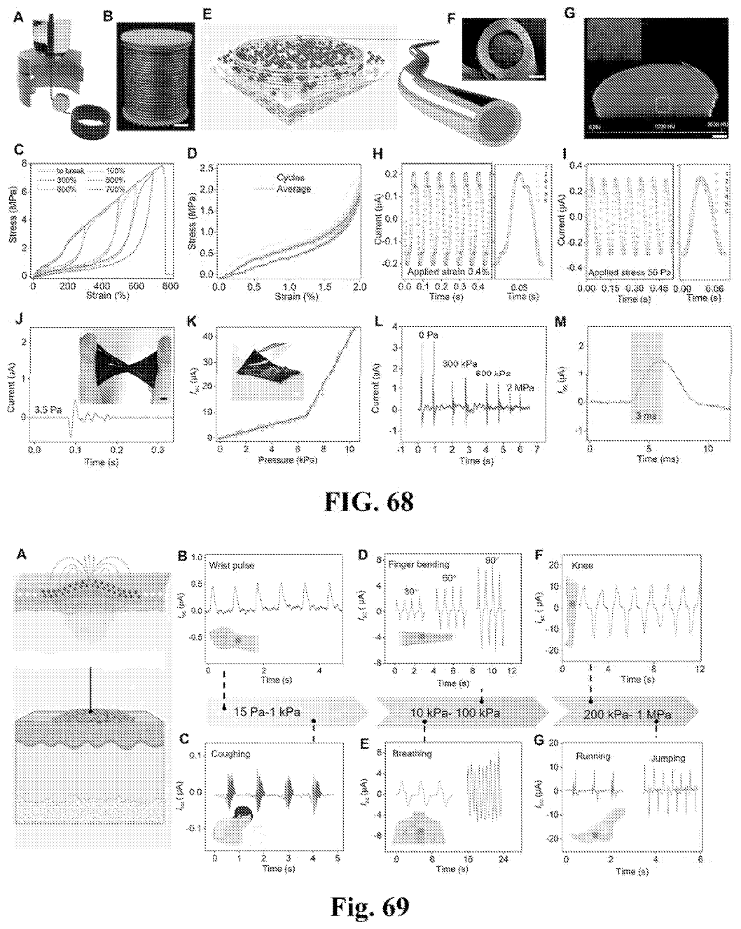

In order to demonstrate the feasibility of employing giant magnetoelastic effect for building up soft-matter electronics, embodiments further coupled the giant magnetoelastic effect with magnetic induction to make an ultra-stretchable pressure sensor. As shown in FIG. 68A, an elastic silicone microfiber was made by thermal-drawing styrene-ethylene-butylene-styrene (SEBS)-covered polyvinyl alcohol (PVA) rods with a diameter of 400 μm. After the PVA was dissolved in water, an elastic hollow channel was obtained. Then, by injecting the liquid metal alloy (74.5% Ga, and 25.5% In by weight) into the elastic hollow channel, the liquid metal microfibers were fabricated (FIG. 68B). The liquid metal microfibers are ultra-elastic with good cyclability. As shown in FIG. 68C, the liquid metal microfibers can be stretched up to 7.8 times of their original length with the Young's modulus of 2.15 MPa.

Moreover, the loading-unloading curves of the strain from 100% to 700% almost overlapped with the tensile curve shown in black color, which indicates a stable mechanical property. It also shows excellent elastic recovery by the tensile test to 200% for 100 times (FIG. 68D). After the first cycle, subsequent cycles almost overlapped with each other, and there was almost no unrecoverable strain. Moreover, the repeatable tensile test from 100% to 700% also overlapped (FIG. 74), which indicates that there is negligible plastic deformation during a tensile test and it is resilient with minimum hysteresis. An ultra-stretchable thin film was made by coupling the soft polymer composite with the thin liquid metal microfibers (FIG. 68E and inset FIG. 68J). The SEM images in FIG. 68F show the cross-section area of the liquid metal microfiber, which confirms that the diameter of the liquid metal microfibers is ˜400 μm. The micro-CT image in FIG. 68G indicates that the liquid metal microfibers were dispersed in the soft polymer composite in a helix structure, which could sensitively capture the magnetic field variation within the soft thin film.

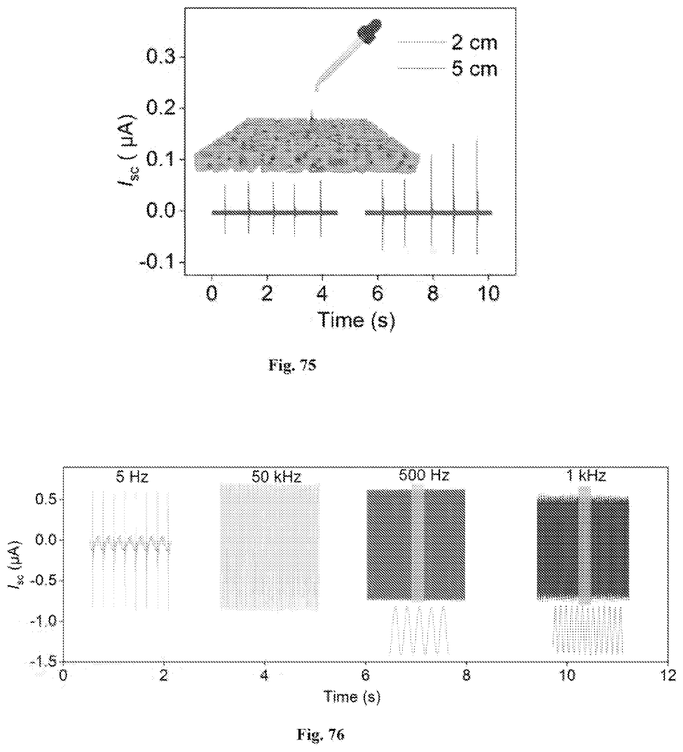

To evaluate the biomechanical sensing performance, a low strain of 0.4% and low pressure of 50 Pa were applied to the magnetoelastic sensor, whose curves are illustrated in FIGS. 68H and 681, respectively. The magnetoelastic sensor response curves from six consecutive loading-unloading cycles almost overlapped with each other, which indicates that there was negligible hysteresis. Importantly, to show the high sensitivity of the magnetoelastic sensor, pressure as low as 3.5 Pa (corresponding to a falling leaf or a drop of water) can be successfully detected (FIG. 68J and FIG. 75). Loading-unloading tests were conducted at 1 Hz frequency with increased applied pressure. As shown in FIG. 68K, the current responds linearly with the increased pressure in the range from 0.06 kPa to 7.52 kPa, and the current increases quicker in the high-pressure range. With a fundamentally new working mechanism based on the giant magnetoelastic effect, the magnetoelastic sensor shows a very wide working range. As demonstrated in FIG. 68L, the sensor can still work under the loading pressures of 0 kPa, 250 kPa, 500 kPa, and 2,000 kPa, of which the working pressure range is ˜20 times larger than that of other sensors based on triboelectric effect, piezoelectric effect, piezoresistive effect, and capacitive. The detailed comparison is illustrated in Table C-1. In addition, the response time of the magnetoelastic sensor is only 3 ms as shown in FIG. 68M. Due to the fast response time, the magnetoelastic sensor also shows its advantages in producing reliable responses to a wide range of pressure frequencies. It exhibits high-response characteristics for high-frequency vibrations up to 1 kHz (FIG. 76), which exceed the human vibration detection range (500 Hz) (S. Chun, A. Hong, Y, Choi, C. Ha, W. Park, A tactile sensor using a conductive graphene-sponge composite. Nanoscale 8, 9185-9192 (2016)). Thus, it is clear that the magnetoelastic sensor exhibits human tactile perception capability

TABLE C-1

| Working | | Sensitivity | Response | | | |
|---|---|---|---|---|---|---|
| Mechanism | Waterproof | range | time | | Stretchability | Ref. |
| Capacitive | No | 0 Pa-25 kPa | 29 | ms (33) | 140% (22) | (22, 23) |
| Piezoresistance | Yes | 0 Pa-100 kPa | 120 | ms, (34) | / | (21, 34) |
| | | | 186 | ms, (35) | | |
| | | | 600 | ms (36) | | |
| Piezoelectric | No | 230 Pa-10 kPa | 21 | ms (38) | 300% (39) | (37-39) |
| Triboeletrical | No | 0 Pa-100 kPa | 100 | ms (40) | 100% (41) | (40, 41) |
| Giant | Yes | 3.5 Pa-2000 kPa | 5 | ms | 550% | This |
| magnetoelastic | | | | | | work |

### C. Self-Powered Biomechanical Activities Monitoring

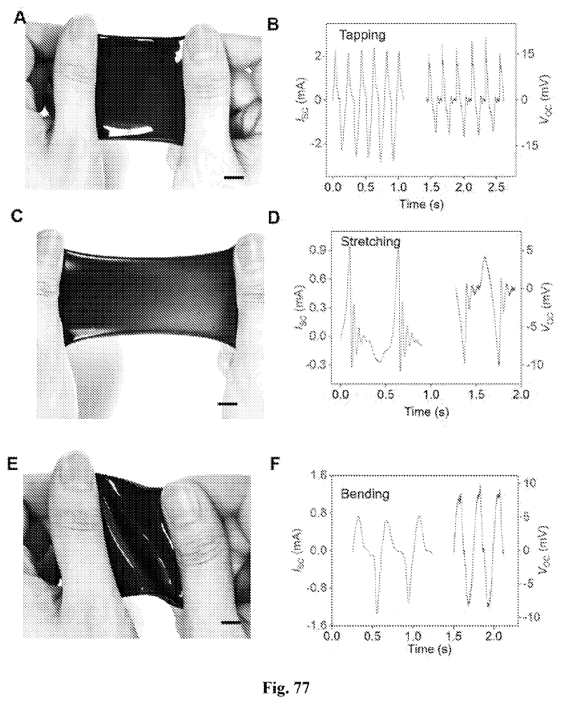

All the physiological signals obtained from the human body are valuable for clinical practice. However, different types of biomechanical activities are associated with different pressure distribution. To perform a whole body biomechanical activities monitoring, a pressure sensor with a wide sensing range is highly desirable. To advance the field development, embodiments develop the stretchable magnetoelastic sensor based on giant magnetoelastic effect. The as-fabricated sensor can be bent, stretched, and tapped, as demonstrated in FIGS. 77A, 77C, and 77E.

Additionally, the magnetoelastic sensor was proved to be sensitive to variety of external pressure under versatile mechanical deformations, in which the electric outputs were characterized and displayed in FIGS. C-77B, 77D, and 77F.

Thus, to demonstrate its versatile applications in human bodies, the magnetoelastic sensor was conformally attached to body parts for a whole body physiological signal and joint movement monitoring, including subtle skin deformation (pulse signal), mid-level skin deformation (finger bending), and substantial joint movement (lower limb). All the signal is tested under heavy perspiration. The working mechanism of the magnetoelastic sensor is shown in FIG. 69A. When it was formally attached on the skin, the subtle deformation of the sensor will induce magnetic field variation within the soft magnetic composite due to the strong magnetomechanical coupling.

Subsequently, the magnetic field variation will be sensitivity obtained by the liquid metal microfibers within the sensor. Consequently, the tiny pressure fluctuations are converted into high-quality electrical signals via the magnetoelastic sensor for further biomedical characterization. Embodiments present the application in human body biomonitoring in sequence of different sensing range. First, the subtle pulse wave was accurately detected by the magnetoelastic sensor (FIG. 69B), which can be used for the assessment of the physical health state of human bodies.

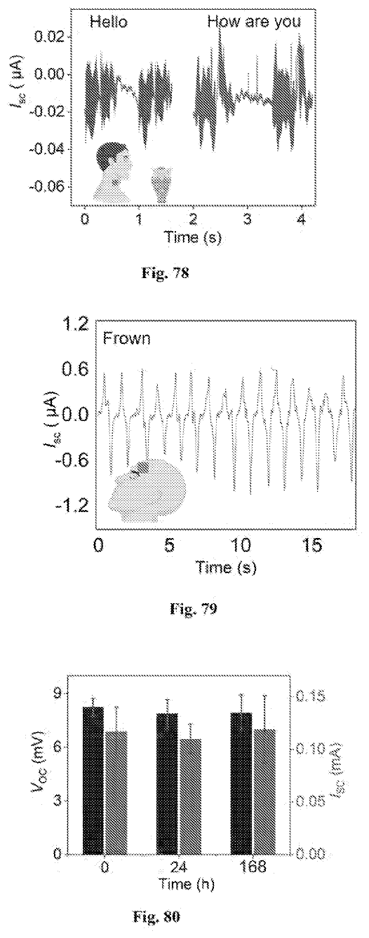

Then, the sensor was attached on the throat, due to its fast response time, the sensor can be used for cough assessment and voice detection (FIG. 69C and FIG. 78). The tiny muscle expression of a frown can also be converted into an electrical current signal (FIG. 79). Third, the sensor was used for mid-level motion detection. As shown in FIG. 69D, the finger bending angle was reflected by the amplitude of the current signal, which is due to the large deformation that causes more magnetic field variation. Respiration is important physiological signal which can be used for breathing detection in those with sleep disorders. As shown in FIG. 69E, the breathing signal was converted into an electrical signal. Moreover, respiratory patterns, such as deep breathing and rapid breathing can clearly be distinguished. Apart from the physiological signals, including sphygmus, breathing, and finger bending, the magnetoelastic sensor can also be applied for substantial joint movement monitoring (lower limb). FIG. 69F demonstrates its application for walking frequency monitoring and FIG. 69G shows the potential for motion state monitoring. Importantly, the humidity-resistance is an indispensable feature for biomonitoring since sweat glands on human skins can perspire up to 3.5 L hour⁻¹ in humans' daily exercise (D. Wendt, L. J. C. van Loon, W. D. Marken Lichtenbelt, Thermoregulation during exercise in the heat. Sports Med. 37, 669-682 (2007)). Therefore, also tested was its chemical stability, and as shown in FIG. 80, there was no obvious decay after the magnetoelastic sensor was immersed in artificial perspiration for 168 hours.

### C. Biocompatibility Characterization and Implantable Biomedical Application

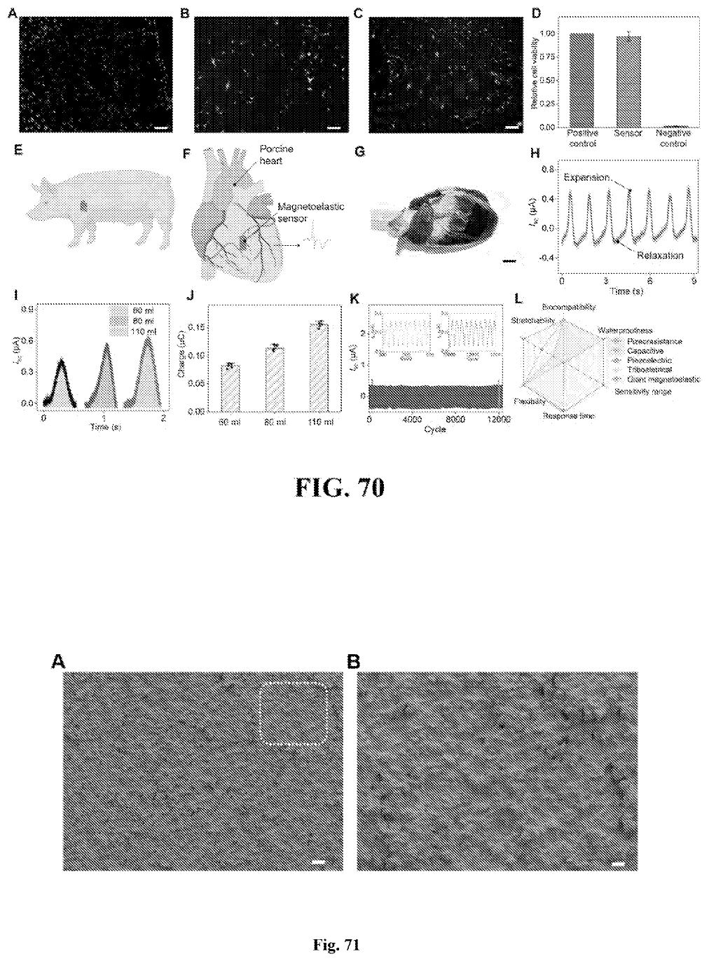

Since there are urgent needs for implantable sensors for applications such as cardiovascular monitoring (Q. Zheng, Q. Tang, Z. L. Wang, Z. Li, Self-powered cardiovascular electronic devices and systems. Nat. Rev. Cardiol. 18, 7-21 (2021)) and orthopaedic rehabilitation (V. Glatt, C. H. Evans, M. J. Stoddart, Regenerative rehabilitation: The role of mechanotransduction in orthopaedic regenerative medicine. J. Orthop. Res. 37, 1263-1269 (2019)), the biocompatibility of the sensor is essential for bioelectronics (J. Deng et al., Electrical bioadhesive interface for bioelectronics. Nat. Mater, 20, 229-236 (2021); C. Dagdeviren et al., Conformal piezoelectric systems for clinical and experimental characterization of soft tissue biomechanics. Nat. Mater. 14, 728-736 (2015); Y. Song, D. Mukasa, H. Zhang, W, Gao, Self-powered wearable biosensors. Acc. Mater. Res. 2, 184-197 (2021); H. Ryu et al., Self-rechargeable cardiac pacemaker system with triboelectric nanogenerators. Nat. Commun. 12, 4374 (2021); Y. S. Choi et al., Fully implantable and bioresorbable cardiac pacemakers without leads or batteries. Nat. Biotechnol., https://doi.org/10.1038/s41587-41021-00948-x (2021)), By: using an in vitro culture of human fibroblasts, the magnetoelastic sensor, based on the giant magnetoelastic effect, was confirmed to be biocompatible. As shown in FIGS. 70A-C, the fluorescent images demonstrate that the cells being cultured on magnetoelastic sensor had undetectable cell death. These results indicate that cell death rarely occurs on the magnetoelastic sensor surface, which is significantly different than that in the negative control group treated with 20% dimethylsulfoxide (DMSO). As shown in FIG. 70D, the quantification of cell viability shows that over 95% of human fibroblasts on the magnetoelastic sensor surface survived after 24 hours, indicating excellent biocompatibility. Thus, embodiments took a further step to test it on ex vivo porcine heart for implantable biomedical application.

Arrhythmias can lead to most of the sudden cardiacal deaths, which will usually influence on endocardial pressure, owning to the abnormal electrical impulses inside heart's chamber (Z. Liu et al., Transcatheter self-powered ultrasensitive endocardial pressure sensor. Adv. Funct. Mater. 29, 1807560 (2019)). Hence, embodiments tested the feasibility of detecting arrhythmias by using magnetoelastic sensor to monitor heart rhythms and stroke volume. The schematic illustration of the ex vivo testing is shown in FIGS. 70E and 70F. The magnetoelastic sensor was placed on a porcine heart (FIG. 70G). The generated cardiac signal from the magnetoelastic sensor was demonstrated in FIG. 70H, in which the signal was clearly illustrated during heart's expansion and relaxation. In addition, the magnetoelastic sensor also detected abnormal heart rhythms caused by arrhythmias such as ventricular fibrillation (FIG. 81). Heart failure is a serious public health problem, in which the heart is incapable to pump sufficient blood to the body. Examples used a different volume of fluid pumping into the porcine heart to mimic systolic cardiac contraction. After implanting the magnetoelastic sensor into the left ventricle, the volume of fluid (60 ml, 80 ml, and 110 ml) pumped into the heart chamber was clearly distinguished as shown in FIG. 70I. In order to statistics analyze the relationship between the electrical signal and the volume of the pumped fluid, the generated electric charges during each cycle was used to characterize the stroke volume (FIG. 70J), which demonstrated linear relationship. Importantly, the mechanical durability of the magnetoelastic sensor is also an important part for the long-term usage of biomedical devices. Thus, the magnetoelastic sensor was loaded-unloaded 12,000 times to characterize its long-term durability, which showed only a 0.01% decay (FIG. 70K). These promising results suggest that the engineered magnetoelastic sensor may facilitate the development of the next generation of implantable biomedical sensors for diagnosis of cardiovascular diseases, such as ventricular fibrillation and ventricular premature contraction.

In order to illustrate the unique advantage of the magnetoelastic sensor, embodiments took a further step to comprehensively compare it with other sensors that are based on the triboelectric, piezoelectric, piezoresistive, and capacitive effect in six performance indexes, including biocompatibility, waterproofness, sensitivity range, response time, flexibility, and stretchability. As shown in FIG. 70L, the magnetoelastic sensor shows advantages in all the six performance indexes (see Table C-1 for more detail). First, it has a wide sensing range from 3.5 Pa to 2,000 kPa, which could match the biomechanical pressure on the whole human body. Secondly, the magnetoelastic sensor is ultra-stretchable and flexible with an ultrafast response time within 5 ms. Also, it is biocompatible. With a collection of advantages, the magnetoelastic sensor showed excellent potential for implantable and wearable bioelectronics.

### C. Discussion

From a scientific point of view, this work uncovers an alternative mechanism to realize a strong magnetoelastic effect in soft systems, different from the traditional magnetoelastic effect found in metal alloys. From a materials point of view, materials with a high magnetomechanical coupling factor and low external magnetic field are highly desirable by the community of magnetoelasticity. The giant magnetoelastic effect provides solutions towards strongly coupled magnetomechanical systems with high magnetomechanical coupling factors without the need for an external magnetic field. Then, the fabricated liquid metal microfibers provided a variety of possibilities in building up biosensors. From the engineering and applications point of view, the giant magnetoelastic effect and the soft sensor enable technology for the application of ultra-wide sensitivity range biosensors with fast response time, in the millisecond range.

It is worth noting that the soft magnetoelastic sensor could have a greater impact beyond bioelectronics demonstrated in this work. For instance, it can provide another approach for inventing soft mechanically gated switches, controls, and memory devices in a remote manner, leading to alternative information communication and technology devices. Also, the giant magnetoelastic effect in the soft system is not restricted to being coupled with magnetic induction for electricity generation. It is foreseen that it can be widely adopted to establish various soft-matter technologies and open doors to many fields. For instance, it can be coupled with the magnetocaloric effect to control the thermodynamics of materials with applied stress to present a mechanical force driven active personal thermoregulation. It can also be coupled with the magneto-optic effect to tune the refractive index of materials in response to applied stress to invent various soft-matter systems for mechanical force induced optical regulation for controlling the transmission, reflection, and absorption of light. It is envisioned that a soft polymer matrix with versatile configurations represents a novel form of soft-matter electronic for energy, medical care, miniaturized robotics, and artificial perception.

### C. Conclusions

In summary, the present Applicant discovered giant magnetoelastic effect in a soft system due to the dipole-dipole interaction, which exhibits a 3.3 times bigger magnetomechanical coefficient than that found in the most commonly used alloys. A wavy chain analytical model based on magnetic dipole-dipole interaction was established to investigate the mechanism of the giant magnetoelastic effect. After coupling the giant magnetoelastic effect with magnetic induction, an ultra-stretchable magnetoelastic sensor was fabricated, which is ultra-stretchable for up to 550%. Due to its unique working mechanism by manipulating dipole alignment, it has an extremely large sensing range from 3.5 Pa to 2,000 kPa (˜20 times larger than that of sensors in other categories), with response time in the millisecond range. Moreover, it has features including minimal hysteresis, responding to high-frequency vibrations, waterproofness, and biocompatibility. Thus, the magnetoelastic sensor was tested on ex vivo porcine heart and conformally attached to the human body for whole body physiological monitoring, providing a promising strategy for implantable sensing and therapeutics.

### C. Example Methods

Fabrication of liquid metal microfibers. Ga (99.99%) and In (99.99%) ingots were purchased from RotoMetals. Liquid metal (74.5% Ga and 25.5% wt % In) was prepared by heating in a muffle furnace (ThermoFisher) at 200° C. for 2 hours. Then, an elastic hollow microfiber was made by thermal drawing styrene-ethylene-butylene-styrene SEBS-covered polyvinyl alcohol rods (Huayang Chemical co., Ltd). After the PVA was dissolved in water, an elastic hollow channel with a diameter of 400 μm was obtained. Then, by injecting the liquid metal into the elastic hollow channel, the liquid metal microfibers are fabricated.

Fabrication of soft polymer composite. The neodymium-iron-boron (NdFeB) nanomagnets (Magnequench) were firstly mixed with Ecoflex 00-30 part A (Smooth-on Inc.). Then, the Ecoflex 00-30 part B (Smooth-on Inc.) was added to the mixture with the same weight as part A. The weight percent of the NdFeB nanomagnets varies from 50 wt % to 75 wt % with the silicone polymer. After mixing thoroughly for 10 minutes to introduce microbubbles, the three-phase mixture was then cured at 60° C. in an oven (ThermoFisher) for 3 hours. The non-magnetized elastomer was magnetized by applying a magnetic pulse (2.655 T) using an impulse magnetizer (IM-10-30, ASC Scientific) to import stable remnant magnetization.

Constructing an ultra-stretchable magnetoelastic sensor. Liquid metal microfibers were placed as a helix structure of different turns into a three dimensional printing mold (Ender Inc.) with 2×2 cm² area. Then, uncured three-phase mixture was poured into a mold. Finally, the molded mixture was cured at 60° C. in an oven (ThermoFisher) for 3 hours. After that, the mixture of Ecoflex 00-30 parts A and B was coated on the sensor surface.

Preparation of artificial perspiration. The artificial perspiration was used to test the chemical stability of the as-fabricated magnetoelastic sensor. To prepare it, 4.65 g NaCl (Sigma Aldrich), 3.87 g 1 M lactic acid solution (Alfa Aesar), 1.80 g Urea (Alfa Aesar), 1.37 g KCl (Sigma Aldrich), 0,756 g NaHCO3 (Sigma Aldrich), 0.546 g 1 M NH₃·H₂O (Sigma Aldrich), 0.175 g Na₂SO₄ (Sigma Aldrich), and 0.0276 g uric acid (Alfa Aesar) were added in 3 L deionized water and mixed for 30 minutes.

Structure characterization. The morphology of the soft polymer composite was characterized by scanning electron microscopy (Zeiss, supra 40VP). The microcomputed tomography (Micro CT) image of the soft polymer composite was scanned at 80 kVp/140 μA with 500 ms exposure using a μCT scanner (HiCT) developed by the Crump Institute for Molecular Imaging at UCLA. The 2D magnetic flux density mapping on the surface of the soft magnetic microfiber was achieved by continuously measuring the magnetic field using the digital Gauss meter (TD8620, Tunkia) mounted on a three axial motion platform. The stress-strain curves were tested at a stretching rate of 0.25 mm s⁻¹ by a dynamic mechanical analyzer (DMA; RSA III). The Young's modulus was calculated via fitting the tested curves with a Neo-Hookean model. The magnetic hysteresis loop was tested by a SQUID magnetometer (MPMS3, Quantum Design).

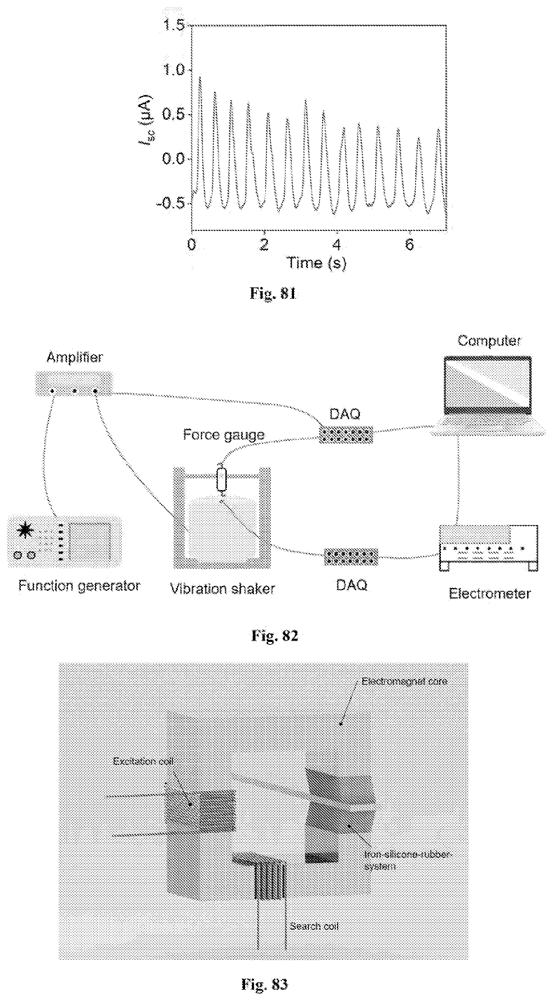

Electrical performance measurement. The voltage and current signals of the as-fabricated magnetoelastic sensor were measured by an electrometer (6514, Keithley). The sensitivity was tested by placing the magnetoelastic sensor on a flat plate, which was connected with an electrodynamic shaker system. It consists of a function generator (AFG1062, Tektronix), a linear power amplifier (PA-151, Labworks Inc.), and an electrodynamic transducer (ET-126HF, Labworks Inc.). The detailed description is shown in FIG. 82.

Biocompatibility test of the magnetoelastic sensor. Human fibroblasts were purchased from ATCC (ATCC® PCS-201-012™) and expanded in fibroblast medium: DMEM (Gibco, 11965), 10% fetal bovine serum (FBS; Gibco, 26140079), and 1% penicillin/streptomycin (GIBCO, 15140122). These fibroblasts were cultured in an incubator at 37° C. and 5% CO₂. For cell viability assay, before seeding cells, the magnetoelastic sensor was plasma-treated for 1 minute and coated with 0.1% gelatin for 1 hour. Then fibroblasts were plated and allowed to attach to the magnetoelastic sensor for 24 hours. The cell viability was assayed by using the PrestoBlue® Cell Viability Reagent (Invitrogen, A13261) according to the manufacturer's protocol. Briefly, cells were incubated with the 10% PrestoBlue Reagent for 2 hours. Results were normalized to control samples (i.e., cells seeded in tissue culture plate). In addition, Live and dead assays were performed by using the LIVE/DEAD™ Cell Imaging Kit (Invitrogen, R37601). Cells were incubated with an equal volume of 2× working solution for 30 minutes at room temperature. Epifluorescence images were collected by using a Zeiss Axio Observer Z1 inverted fluorescence microscope and analyzed using Image J software. In all experiments, cells cultured in tissue culture dishes were used as positive controls, and cells treated with 20% DMSO were used as negative controls.

Ex vivo test on a porcine heart. The porcine heart was connected with an air pump to control the heart beating frequency. Different volume of fluid pumping into the porcine heart to mimic systolic cardiac contraction.

Human Subject Study. The magnetoelastic sensor used for wearable cardiovascular monitoring was performed using human subjects in compliance with all the ethical regulations under a protocol (ID: 20-001882) that was approved by the Institutional Review Board (IRB) at University of California, Los Angeles. All participating subjects belonged to University of California, Los Angeles and were provided informed consent before participation in the study:

### C: Supplementary Note 1: Detailed Comparison of the Magnetorheological Elastomers.

The concept of magnetorheological elastomers was first proposed in 1983 when Rigbi and Jilken studied an elastomer filled with soft ferrite (Z. Rigbi, L. Jilken, The response of an elastomer filled with soft ferrite to mechanical and magnetic influences. J. Magn, Magn. Mater. 37, 267-276 (1983)). In the following years, the research focus of magnetorheological elastomers has been placed on their magnetostriction property of changing shapes or dimensions under an external magnetic field (X. Guan, X. Dong, J. Ou, Magnetostrictive effect of magnetorheological elastomer. J. Magn. Magn. Mater. 320, 158-163 (2008); H. X. Deng, X. L. Gong, Adaptive tuned vibration absorber based on magnetorheological elastomer. J. Intell. Mater. Syst. Struct. 18, 1205-1210 (2016)). As a result, their applications have been mainly limited to adaptive vibration isolators and controllers for civil and mechanical engineering. In recent years, their tunable, mechanical properties, such as stiffness and shear modulus under an applied magnetic field, have received great attention (R. Zhao, Y. Kim, S. A. Chester, P. Sharma X. Zhao, Mechanics of hard-magnetic soft materials. J. Mech. Phys. Solids 124, 244-263 (2019)). By manipulating magnetic domains of hard magnetorheological elastomers in an untethered manner, magnetic soft robots with complex shape-transformability have been widely reported (Y. Kim, H. Yuk, R. Zhao, S. A. Chester, X. Zhao, Printing ferromagnetic domains for untethered fast-transforming soft materials. Nature 558, 274-279 (2018); Y. Kim, G. A. Parada, S. Liu, X. Zhao, Ferromagnetic soft continuum robots. Science Robotics 4, eaax7329 (2019)).

The inverse effect of magnetostriction, however, is previously ignored although it has great potential for soft-matter electronics. It is believed that the mechanically-induced magnetic properties change has been rarely reported in previous research. As set forth above, the present Applicant discovered the giant magnetoelastic effect in soft systems with a more remarkable magnetomechanical coupling factor than bulk metal alloys such as TbₓDy₁₋ₓ Fe₂ (Terfenol-D) (Q. Su, J. Morillo, Y, Wen, M. Wuttig, Young's modulus of amorphous Terfenol-D thin films. Journal of Applied Physics 80, 3604-3606 (1996)) and GaₓFe₁₋ₓ (Galfenol) (Z. Deng, M. J. Dapino, Review of magnetostrictive materials for structural vibration control. Smart Materials and Structures 27, 113001 (2018); S. Datta, J. Atulasimha, C. Mudivarthi, A. B. Flatau, Stress and magnetic field-dependent Young's modulus in single crystal iron-gallium alloys. J. Magn. Magn. Mater. 322, 2135-2144 (2010)). It is different from the traditional magnetoelastic effect in metal alloys since this effect in soft systems results from the changed particle arrangement in soft systems. Moreover, the discovered giant magnetoelastic effect in the present soft polymer matrix is also distinct from the pseudo-magnetoelastic effect in the iron-silicone-rubber system (G. Diguet, G. Sebald, M. Nakano, M. Lallart, J.-Y. Cavaillé, Magnetic particle chains embedded in elastic polymer matrix under pure transverse shear and energy conversion. J. Magn. Magn. Mater. 481, 39-49 631 (2019)) for three reasons.

First, the magnetoelastic effect in the iron-silicone-rubber system requires a magnetic field of around 0.2-0.3 T, which equals 2000 to 3000 oe. Such a static magnetic field is even larger than that typically required by the conventional rigid counterpart and therefore needs to be supplied by a cumbersome electromagnet, which hinders its possibility of practical applications especially in the field of wearable and implantable bioelectronics (Supplementary FIG. 83). It is also clearly shown that without an external magnetic field, the iron-silicone-rubber system did not exhibit any magnetoelastic effect at all. Therefore, instead of a true magnetoelastic effect, the studied effect in the iron-silicone-rubber system is more appropriate to be called pseudo-magnetoelastic effect. Furthermore, the use of an electromagnet will bound and prevent the leakage of the magnetic flux, which inevitably overestimates the performance of the iron-silicone-rubber system.

Secondly, the pseudo-magnetoelastic effect is studied by applying shear strain using a steel blade whereas the giant magnetoelastic effect is studied by applying uniaxial stress (strain), which is much more common in human biomechanical motions than shear strain. Therefore, the present studies are more universal for bioelectronics applications.

Third and most importantly, the working mechanisms of the pseudo-magnetoelastic effect and giant magnetoelastic effect are different. For the pseudo-magnetoelastic effect, the magnetic flux density change is caused by the change of apparent permeability (susceptibility) under an external magnetic field. As a result, there is an optimal magnetic field around 0.2 T to achieve the best output performance. When the applied magnetic field is 0 T, there is no observable magnetoelastic effect. When the applied magnetic field is high enough (˜0.7 T) to saturate the iron particle, the magnetoelastic effect diminishes because the permeability of the iron-silicone-silicone system will not change in such a situation. On the contrary, the giant magnetoelastic effect in the soft polymer matrix relies on the arrangement change of readily magnetized nanomagnets. It does not require an external magnetic field and, in principle, will not be affected by the magnetization saturation of the microparticles.

As a result, the theoretical model of the present system is significantly different from the one used in the iron-silicone rubber system. The theory used in the iron-silicone-rubber system cannot explain the magnetic flux decrease of the present system under uniaxial stress, since in this case, the particle interaction should increase with decreased particle inter-distance. By adopting the wavy chain microstructure and demagnetizing factor, the present theoretical model was able to explain the observed negative giant magnetoelastic effect.

### C: Supplementary Note 2. Theoretical Explanation of the Wavy Chain Analytical Model

The giant magnetoelastic effect in the soft polymer matrix can be described by using the wavy chain model with dipole-dipole interaction and demagnetizing factor. For the dipole-dipole interaction, it is assumed that under impulse magnetization, the nanomagnets, which can be approximately regarded as single magnetic dipoles (T. Borbáth, S. Günther, D. Yu Borin, T. Gundermann, S. Odenbach, XμCT analysis of magnetic field-induced phase transitions in magnetorheological elastomers. Smart Mater. Struct. 21, 105018 (2012)), align in a zig-zag wavy chain structure as illustrated in FIG. 68i. It is further assumed that dipole-dipole interaction exists only inside the wavy chain according to previous literature (Y. Han, W. Hong, L. E. Faidley, Field-stiffening effect of magneto-rheological elastomers. Int. J. Solids Struct. 50, 2281-2288 (2013)). To simplify the calculation, it is considered that each magnetic dipole has a vertical magnetization M, which is reasonable, owing to the system symmetry. For the demagnetizing factor, embodiments approximate the wavy chain as a square rod with an aspect ratio of a. Based on the above assumption, the vertical magnetic field of a nanomagnet on the surface of the soft polymer matrix can be approximately expressed as,

$${H}_{⊥} \approx \frac{1}{( 2 a + 1 ) {λ}^{1.5 k}} M + \frac{{r}^{3} M}{3 {λ}^{3} {h}^{3}} ( 0.3006 - f ( \frac{1}{h {λ}^{1.5}} ) ) \qquad ( 1 )$$

$$f ( \frac{1}{h {λ}^{1.5}} ) = {\sum }_{n = 1}^{∞} ( {( \frac{1}{h {λ}^{1.5}} )}^{2} - 2 {( 2 n - 1 )}^{2} ) {( {( \frac{1}{h {λ}^{1.5}} )}^{2} + {( 2 n - 1 )}^{2} )}^{- 5 / 2} \qquad ( 2 )$$

where λ is the principal stretch in the compress direction and r is the radius of the nanomagnets. h and l denote the vertical and horizontal distances between two adjacent magnetic dipoles in the wavy chain, respectively. 0.3006−f(x) is the dipole alignment factor describing the contribution of all other magnetic dipoles to the vertical magnetic field of the single dipole on the surface of the soft polymer matrix in the wavy chain. 1/2α+1 represents the demagnetizing factor of the wavy chain structure. k represents a constant characterizing the influence of nonideality, neighboring chain-chain interaction, and macroscopic shape effect to the demagnetizing factor under compressive deformation. Then the variation of the magnetic field due to elastic deformation of the soft magnetic system can be expressed as below

$${H}_{1 ⊥} / {H}_{0 ⊥} = \frac{\frac{1}{( 2 a + 1 ) {λ}^{1.5 k}} + \frac{{r}^{3}}{3 {λ}^{3} {h}^{3}} ( 0.3006 - f ( \frac{1}{h {λ}^{1.5}} ) )}{\frac{1}{( 2 a + 1 )} + \frac{{r}^{3}}{3 {h}^{3}} 0.3006 \to f ( \frac{l}{h} ) )} \qquad ( 3 )$$

With estimated values of a=105, r=2.5 μm, h=13.5 μm, 1=14.85 μm, and G=630 kPa for the soft polymer matrix, and the compressive stress s through an incompressible Neo Hookean model below,

$$s = G ( λ - 1 / {λ}^{2} ) \qquad ( 4 )$$

When k equals 0.05, the wavy chain model accurately captures its magnetic field variation in response to the compressive stress changing from 0 to 2000 kPa, which is well consistent with the experimental observation. It is worth noting that the derived H0⊥/H1⊥ only approximately represents the ideal case in which the edging effect and the shape of soft polymer matrix were not considered. The introduction of k into equation 1 is based on three reasons: 1. The ideal rectangular rod with uniform magnetization only roughly approximates the wavy chain structure. 2. The influence of neighboring wavy chains on the demagnetizing factor of a single wavy chain cannot be simply ignored. 3. The macroscopic shape effect determines that the overall demagnetizing factor will not change significantly based on the macroscopic shape of the soft polymer matrix. This macroscopic effect needs to be unified with the wavy chain microstructure inside the soft polymer matrix.

It should also be mentioned that the theoretical consideration is based on a lot of assumptions and simplifications. Therefore, it only roughly approximates the experimental results. A more sophisticated theory should be developed in the future to better address the magnetoelastic effect in soft magnetic systems.

According to certain alternative and/or additional third aspects of the present embodiments, it was discovered that the giant magnetoelasticity in soft matter can achieve up to five times enhancement of magnetomechanical coupling factors than traditional rigid metal-based counterparts. To understand this phenomenon, a wavy chain analytical model based on the magnetic dipole-dipole interaction in the soft matter was established, fitting well to the experimental observation. Then explored was this discovery in electronic textiles and coupled it with Faraday's law of induction to invent a textile magnetoelastic generator (MEG) for biomechanical-to-electrical energy conversion. The developed textile MEG demonstrates an intrinsic waterproof property, an ultralow internal impedance around ˜20Ω, and a high short-circuit current density of 1.37 mA/cm², which is about four orders of magnitude higher than other textile counterparts for biomechanical energy conversion. Meanwhile, assisted by machine learning, the textile MEG was demonstrated as a self-powered textile respiration sensor. It could continuously monitor the respiratory biomechanical activities on heavy perspiration skin without any encapsulation, allowing a timely diagnosis of the respiration abnormalities in a wearable manner. It is foreseen that the discovery of giant magnetoelasticity can be extended to wide-range soft-matter systems, emerging as a compelling approach to developing functional electronic textiles for energy, sensing, and therapeutic applications.

### D. Textile Magnetoelastic Generator for Energy Harvesting and Health Monitoring

### D. Summary

According to a fourth aspect, a textile MEG was developed as a new mechanism for biomechanical energy harvesting. The textile MEG features an ultrahigh current density, ultralow internal impedance, and an intrinsic waterproof property. The textile magnetoelastic generator emerges as a new form of electronics textiles with intrinsic waterproof capability.

As set forth above, the present Applicant discovered the giant magnetoelasticity in soft matter with up to five times enhancement of magnetomechanical coupling factors than traditional rigid metal-based counterparts. To understand this phenomenon, a wavy chain analytical model based on the magnetic dipole-dipole interaction in the soft matter was established, fitting well to the experimental observation. Then explored was this discovery in electronic textiles and coupled it with Faraday's law of induction to invent a textile magnetoelastic generator (MEG) for biomechanical-to-electrical energy conversion. The developed textile MEG demonstrates an intrinsic waterproof property, an ultralow internal impedance around ˜20Ω, and a high short-circuit current density of 1.37 mA/cm², which is about four orders of magnitude higher than other textile counterparts for biomechanical energy conversion. Meanwhile, assisted by machine learning, the textile MEG was demonstrated as a selfpowered textile respiration sensor. It could continuously monitor the respiratory biomechanical activities on heavy perspiration skin without any encapsulation, allowing a timely diagnosis of the respiration abnormalities in a wearable manner. It is foreseen that the discovery of giant magnetoelasticity can be extended to wide-range soft-matter systems, emerging as a compelling approach to developing functional electronic textiles for energy, sensing, and therapeutic applications.

### D. Introduction

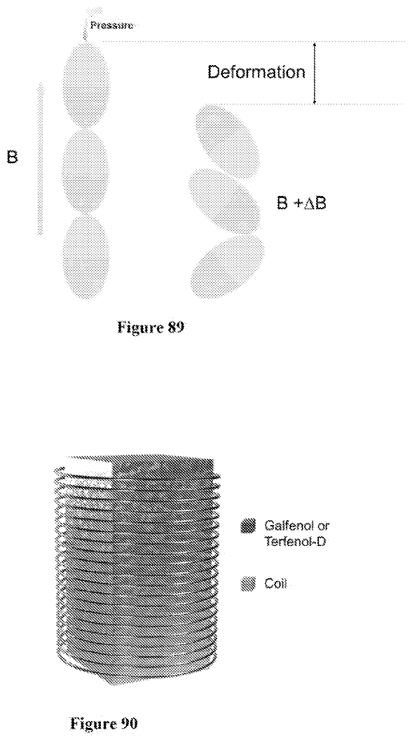

Conventional magnetoelasticity, defined as the change in magnetic property in certain materials under mechanical deformation (FIG. 89), has been observed in rigid metal alloys such as TbₓDy₁₋ₓFe₂ (Terfenol-D) and GaₓFe₁₋ₓ (Galfenol) for building vibration control (Eem, S., Jung, H., and Koo, J. (2011), Application of MR elastomers for improving seismic protection of base-isolated structures. IEEE Trans. Magn, 47, 2901-2904; Deng, Z., and Dapino, M. J. (2018). Review of magnetostrictive materials for structural vibration control. Smart Mater. Struct. 27, 113001) as sketched in FIG. 90, it holds a bulky, heavy and rigid structure. The conventional magnetoelasticity in metal alloys was rarely adopted to build devices for interfacing with human body since: 1) Human skin and tissues are soft and not easily adapted to these hard metal alloys (Su, Q., Morillo, J., Wen, Y., and Wuttig, M. (1996). Young's modulus of amorphous Terfenol-d thin films. J. Appl. Phys. 80, 3604-3606). 2) The optimal magnetomechanical coupling efficiency on these rigid metal alloys always requires the pressure of several mega pascals, beyond the range of biomechanical stress. 3) These rigid metal alloys require external magnetic fields of up to 1000 Oe when implementing functionality (Davino, D., Giustiniani, A., and Visone, C. (2012). The piezo-magnetic parameters of Terfenol-d: An experimental viewpoint. Physica B Condens. Matter 407, 1427-1432). Textiles have been concomitant of human civilization for thousands of years (Chen, G., Li, Y., Bick, M., and Chen, J. (2020). Smart textiles for electricity generation. Chem, Rev. 120, 3668-3720). Nowadays, advances in materials science and nanotechnology facilitate the infusion of electronics and textiles, which has been a compelling approach to realize electronic textiles (e-textiles) with additional functions while maintaining their breathability, biocompatibility, and wearing comfort (Su, Y., Chen, C., Pan, H., Yang, Y., Chen, G., Zhao, X., Li, W., Gong, Q., Xie, G., Zhou, Y, et al. (2021). Muscle fibers inspired high-performance piezoelectric textiles for wearable physiological monitoring. Adv. Funct. Mater. 31, 2010962; Fang, Y., Chen, G., Bick, M., and Chen, J. (2021). Smart textiles for personalized thermoregulation. Chem. Soc. Rev. DOI: 10.1039/D1CS00003A; Chen, G., Fang, Y., Zhao, X., Tat, T., and Chen, J. (2021). Textiles for learning tactile interactions. Nat. Electron. 4, 175-176; Zhou, Z., Chen, K., Li, X., Zhang, S., Wu, Y., Zhou, Y., Meng, K., Sun, C., He, Q., Fan, W., et al. (2020). Sign-to-speech translation using machine-learning-assisted stretchable sensor arrays. Nat. Electron. 3, 571-578; Zhang, N., Huang, F., Zhao, S., Lv, X., Zhou, Y., Xiang, S., Xu, S., Li, Y, Chen, G., Tao, C., et al. (2020). Photo-rechargeable fabrics as sustainable and robust power sources for wearable bioelectronics. Matter 2, 1260-1269; Meng, K., Zhao, S., Zhou, Y., Wu, Y., Zhang, S., He, Q., Wang, X., Zhou, Z., Fan, W., Tan, X., et al. (2020). A wireless textile-based sensor system for self-powered personalized health care. Matter 2, 896-907; Chen, J., Huang, Y., Zhang, N., Zou, H., Liu, R. Tao, C., Fan, X., and Wang, Z. L. (2016). Micro-cable structured textile for simultaneously harvesting solar and mechanical energy. Nat. Energy 1, 16138). In view of this, abundant e-textiles have been developed to perform various applications on the human body, such as energy harvesting/storage (Yin, L., Kim, K. N., Lv, J., Tehrani, F., Lin, M., Lin, Z., Moon, J.-M., Ma, J., Yu, J., Xu, S., and Wang, J. (2021). A self-sustainable wearable multi-modular e-textile bioenergy microgrid system. Nat. Commun. 12, 1542; Xu, L., Fu, X., Liu, F., Shi, X., Zhou, X Liao, M., Chen, C., Xu, F., Wang, B., Zhang, B., and Peng, H. (2020). A perovskite solar cell textile that works at −40 to 160° C. Journal of Materials Chemistry A 8, 5476-5483; Ding, T. Chan, K. H., Zhou, Y., Wang, X-Q., Cheng, Y., Li, T., and Ho, G. W, (2020). Scalable thermoelectric fibers for multifunctional textile-electronics. Nat. Commun. 11, 6006), sensing (Chen, G., Au, C., and Chen, J. (2021). Textile triboelectric nanogenerators for wearable pulse wave monitoring. Trends Biotechnol. https://doi.org/10.1016/j.tibtech.2020.1012.1011; Wang, L., Xie, S., Wang, Z., Liu, F., Yang, Y., Tang, C., Wu, X., Liu, P., Li, Y., Saiyin, H., et al. (2020). Functionalized helical fibre bundles of carbon nanotubes as electrochemical sensors for long-term in vivo monitoring of multiple disease biomarkers. Nat. Biomed. Eng. 4, 159-171; Luo, Y., Li, Y., Sharma, P., Shou, W., Wu, K., Foshey, M., Li, B., Palacios, T., Torralba, A., and Matusik, W. (2021). Learning human-environment interactions using conformal tactile textiles. Nat. Electron. 4, 193-201), therapy (Jeong, S.-H., Lee, Y., Lee, M.-G., Song, W. J., Park, J.-U., and Sun, J.-Y. (2021). Accelerated wound healing with an ionic patch assisted by a triboelectric nanogenerator. Nano Energy 79, 105463; Zhao, X., Wang, L.-Y., Tang, C.-Y., Zha, X.-J., Liu, Y., Su, B.-H., Ke, K., Bao, R.-Y., Yang, M.-B., and Yang, W. (2020). Smart Ti3C2Tx MXene fabric with fast humidity response and joule heating for healthcare and medical therapy applications. ACS Nano 14, 8793-8805; Mostafalu, P., Kiaee, G., Giatsidis, G., Khalilpour, A. Nabavinia, M., Dokmeci, M. R., Sonkusale, S., Orgill, D. P., Tamayol, A., and Khademhosseini, A. (2017). A textile dressing for temporal and dosage controlled drug delivery. Adv. Funct. Mater. 27, 1702399), display (Shi, X., Zuo, Y., Zhai, P., Shen, J., Yang, Y., Gao, Z., Liao, M. Wu, J., Wang, J., Xu, X., et al. (2021). Large-area display textiles integrated with functional systems. Nature 591, 240-245; Song, S., Song, B., Cho, C.-H., Lim, S. K., and Jeong, S. M. (2020). Textile-fiber-embedded multiluminescent devices: A new approach to soft display systems. Mater, Today 32, 46-58; Zhang, Z., Cui, L., Shi, X., Tian, X., Wang, D., Gu, C., Chen, E., Cheng, X., Xu, Y., Hu, Y., et al. (2018). Textile display for electronic and brain-interfaced communications. Adv. Mater. 30, 1800323) and even computation (Loke, G., Khudiyev, T., Wang, B., Fu, S., Payra, S., Shaoul, Y., Fung, J., Chatziveroglou, L., Chou, P.-W., Chinn, I., et al. (2021). Digital electronics in fibres enable fabric-based machinelearning inference. Nat. Commun. 12, 3317; Loke, G., Alain, J., Yan, W., Khudiyev, T., Noel, G., Yuan, R., Missakian, A., and Fink, Y. (2020). Computing fabrics. Matter 2, 786-788; Xu, X., Zhou, X., Wang, T., Shi, X., Liu, Y., Zuo, Y., Xu, L., Wang, M., Hu, X., Yang, X., et al. (2020). Robust DNA-bridged memristor for textile chips. Angew. Chem. Int. Ed. 59, 12762-12768). Among them, biomechanical energy conversion textiles have attracted widerange of research interests, since biomechanical motions on the human body are sustainable, pervasive, and easy to obtain (Riemer, R., and Shapiro, A. (2011). Biomechanical energy harvesting from human motion: Theory, state of the art, design guidelines, and future directions. J. Neuroeng. Rehabil. 8, 22). Currently, widely adopted e-textiles for biomechanical energy conversion are based on the triboelectric effect (Fan, F.-R., Tian, Z.-Q., and Lin Wang, Z. (2012). Flexible triboelectric generator. Nano Energy 1, 328-334) and piezoelectric effect (Wang, Z. L., and Song, J. (2006). Piezoelectric nanogenerators based on zinc oxide nanowire arrays. Science 312, 242-246) which was proved to be well-conformal to the human skin for wearable electricity generation. Owing to their capacitive power generation principle via manipulating the electric dipoles at the materials interfaces, the wide-range adoption of such technologies is largely shadowed by a low current density (in the order of 100 nA/cm²) and a high internal impedance (in the order of megaohms) (Xiong, J., Cui, P., Chen, X., Wang, J., Parida, K., Lin, M.-F., and Lee, P. S. (2018). Skintouch-actuated textile-based triboelectric nanogenerator with black phosphorus for durable biomechanical energy harvesting. Nat. Commun. 9, 4280; Mokhtari, F., Spinks, G. M., Fay, C., Cheng, Z., Raad, R., Xi, J., and Foroughi, J. (2020). Wearable electronic textiles from nanostructured piezoelectric fibers. Adv. Mater. Technol. 5, 1900900). Moreover, the generated current relies on the electric dipoles transfer at the materials interfaces, which is vulnerable to the perspiration and ambient humidity, severely limiting their practical on-body applications (Wang, Z. L., and Wang, A. C. (2019). On the origin of contact-electrification. Mater. Today 30, 34-51).

As described above, Applicant discovered the giant magnetoelasticity in a soft matter and achieved a magnetomechanical coupling factor up to 6.77×10-8 T/Pa without the needs of external magnetic field, which is up to five times larger than that of traditional rigid metal-based counterparts (Liu, J., Jiang, C., and Xu, H. (2012). Giant magnetostrictive materials. Sci. China Technol. Sci. 55, 1319-1326) (Table S1). The soft matter with giant magnetoelasticity demonstrates a strain of up to 500% and a Young's modulus as low as 166.2 kPa, which is well comparable to the human tissue and skin.

TABLE S1

| | Magneto- | | | |
|---|---|---|---|---|
| | mechanical | | | |
| | coupling | Young's | Applied | External |
| | factor | modulus | pressure | magnetic |
| Systems | (T/Pa) | (kPa) | range (kPa) | field |
| Terfenol-D | 1.36 × 10^{-8 2} | 1.2 × 10^{8 4} | Megapascal ² | Required ³ |
| Galfenol | 3.85 × 10^{-8 3} | 7.6 × 10^{7 5} | Megapascal ⁴ | Required ³ |
| The present | 6.77 × 10⁻⁸ | 433.3 | Kilopascal | Not |
| work | | | | Required |

A wavy chain analytical model was established to explain the giant magnetoelasticity in the soft matter, which is well consistent with the experimental observation. To demonstrate practicability, the giant magnetoelasticity was further coupled with magnetic induction to develop a textile magnetoelastic generator (MEG) as fundamentally new working mechanism for biomechanical energy conversion. Externally applied pressure on the textile MEG could strongly alter its magnetic flux density and a high current would be induced in the textile coil, which demonstrated a high short-circuit current (Isc) density of 1.37 mA/cm² and a low internal impedance of ˜20Ω.

This current output is about four orders of magnitude higher than that of the triboelectric effect (Xu, F., Dong, S., Liu, G., Pan, C., Guo, Z. H., Guo, W., Li, L., Liu, Y., Zhang, C., Pu, X., and Wang, Z. L. (2021). Scalable fabrication of stretchable and washable textile triboelectric nanogenerators as constant power sources for wearable electronics. Nano Energy 88, 106247) and piezoelectric effect (Zhang, C., Fan, W., Wang, S., Wang, Q., Zhang, Y., and Dong, K. (2021). Recent progress of wearable piezoelectric nanogenerators. ACS Appl. Electron. Mater. 3, 2449-2467) based textile biomechanical energy harvesting counterparts. Meanwhile, textile MEGs are fully waterproof without encapsulation because the magnetic fields can pass through water molecules with negligible intensity loss. Therefore, textile MEGs were developed into self-powered sensors for wearable respiratory monitoring with heavy perspiration. With an optimal sensitivity of 0.27 mA/kPa, signal-to-noise ratio of 61.8 dB, and response time of 15 ms, this textile MEG-based sensor can continuously monitor the strength, frequency, and patterns of various respiratory activities. Assisted by a random forest-based machine learning algorithm, respiration abnormalities can be continuously recognized, such as cough and rapid breathing, hence allowing a timely diagnosis of breath-related diseases. The discovered giant magnetoelasticity in soft matter is a new form of mechanical to magnetic conversion, and the invented textile MEG is bringing a fundamentally new working mechanism to the community of biomechanical energy conversion. These advancements are expected to make a splash in constructing human-body-centered e-textiles for personalized healthcare.

### D. Results

### Design of Soft Matter and Textile MEGs

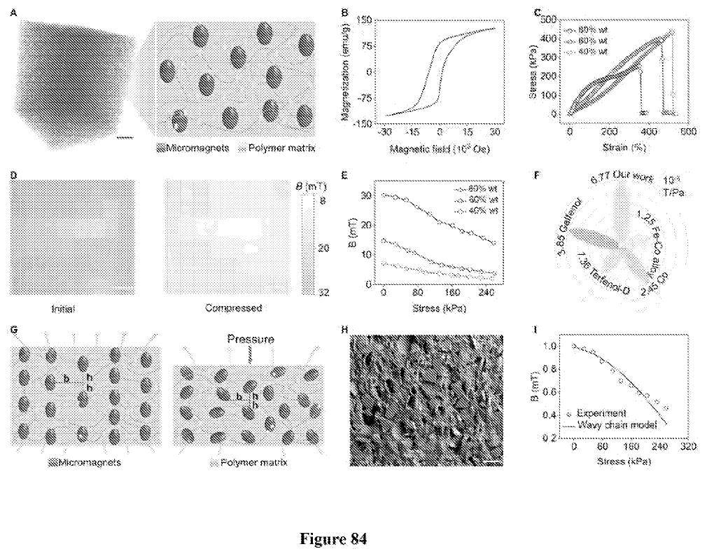

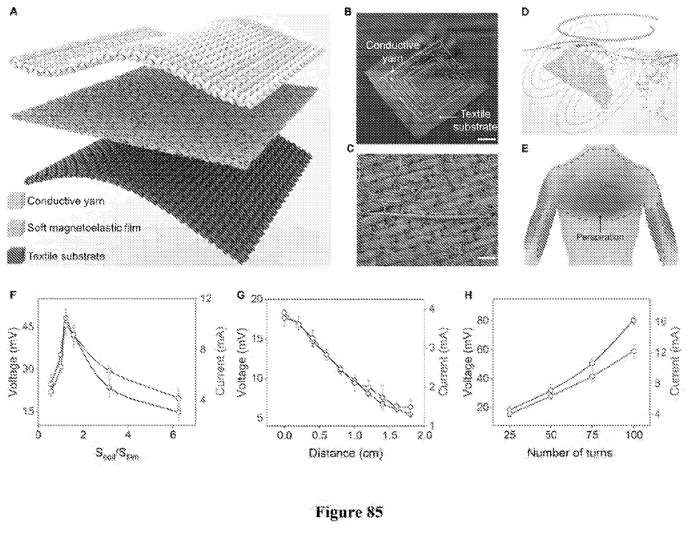

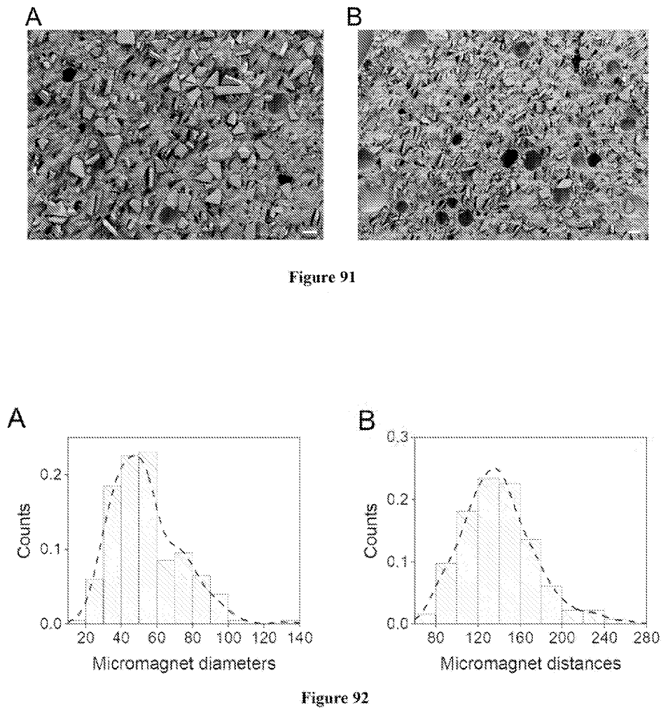

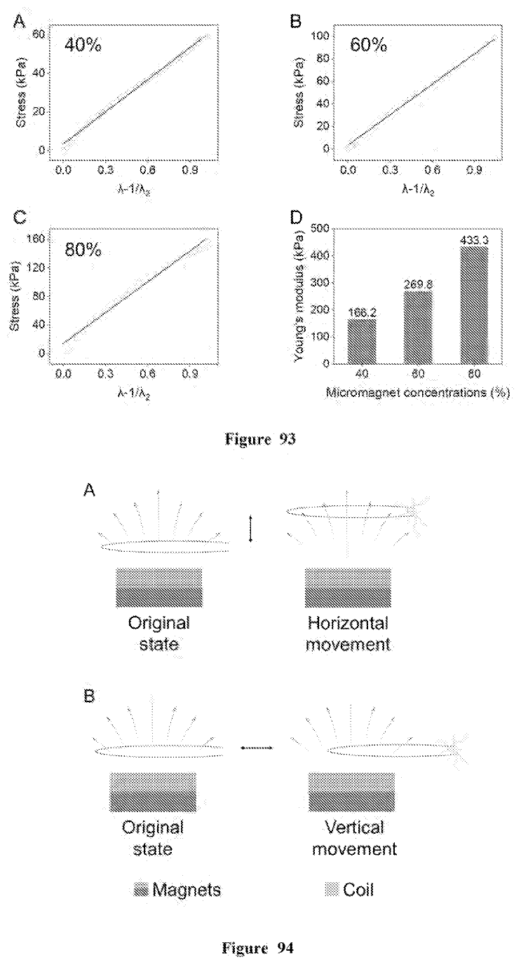

Embodiments observed the giant magnetoelasticity in a soft magnetoelastic film consisting of micromagnets and porous polymer matrix (FIG. 84A). After being subjected to an impulse magnetization, the possible rotation and movement of micromagnets in the polymer matrix construct a chain-like arrangement (Stolbov, O. V., Raikher, Y. L., and Balasoiu, M. (2011). Modelling of magnetodipolar striction in soft magnetic elastomers. Soft Matter 7, 8484-8487), maintaining a large retentivity of 83 emu/g (FIG. 84B). According to the scanning electron microscopy (SEM) (FIG. 91) and the micro-computed tomography (Micro-CT) (Video S1), these micromagnets are evenly distributed in the porous matrix. These micromagnetic particles have a mean diameter of 53.7 μm with a standard deviation of 18.8 μm, and a mean interparticle distance of 139.6 μm with a standard deviation of 34.0 μm (FIG. 92). This unique structure gives the soft magnetoelastic film outstanding mechanical properties that favor the further on-body biomechanical-to-electrical energy conversion, such as the strain of up to 500% and a Young's modulus as low as 166.2 kPa (FIG. 84C and FIG. 93). FIG. 84D shows the magnetic flux density mappings of the soft magnetoelastic film under the initial and compressed states. Under an applied pressure of 257 kPa, the magnetic flux density of the soft magnetoelastic film decreased significantly up to 50%. The relative magnetic flux density decrease of the present soft magnetoelastic film rivals the traditional magnetoelastic system (Diguet, G., Sebald, G., Nakano, M., Lallart, M., and Cavaillé, J.-Y. (2019). Magnetic particle chains embedded in elastic polymer matrix under pure transverse shear and energy conversion. J. Magn. Magn. Mater. 481, 39-49), which needs an extremely high uniaxial stress of more than 10 MPa (Deng, Z. (2015). Nonlinear modeling and characterization of the villari effect and modelguided development of magnetostrictive energy harvesters and dampers. (The Ohio State University)). On this basis, investigated were the values of magnetic flux density variation with different micromagnet concentrations. As shown in FIG. 84E, under a constant stress, the soft magnetoelastic film with 80 wt % micromagnet concentration has the largest values of magnetic flux density variation of 16 mT compared to those with 60 wt % (10.9 mT) and 40 wt % (5.1 mT) micromagnets. More importantly, this soft magnetoelastic film delivers a magnetomechanical coupling factor of 6.77×10⁻⁸ T/Pa without the reliance of an external magnetic field, which is five times larger than that of the traditional magnetoelasticity in the rigid system (FIG. 84F). However, a further increase in loading of the micromagnets could decrease the stretchability (from 500% to 350%) and increase the Young's modulus (from 166.2 kPa to 433.3 kPa) of the soft magnetoelastic film (FIG. 93). Considering this trade-off, the soft magnetoelastic film with 80 wt % micromagnets was picked up for further study

In order to investigate the fundamental science behind the giant magnetoelasticity established was a wavy chain model (Zhou, Y., Zhao, X., Xu, J., Fang, Y., Chen, G., Song, Y Li, S., and Chen, J. (2021). Giant magnetoelastic effect in soft systems for bioelectronics. Nat. Mater. 20, 1300). Without the external pressure, these micromagnets inside the soft magnetoelastic film are well aligned as a wavy chain structure to maintain a stable status after the impulse magnetization (FIG. 84G). When an external pressure is applied on the soft magnetoelastic film, the pressure penetrating the polymer matrix could provide a constant energy for these micromagnetic particles to move and rotate, thus decreasing the magnetic flux density.

In addition, the relationship between the vertical magnetic field H⊥ and principal stretch λ could be expressed as:

$${H}_{⊥} \approx - \frac{1}{( 2 a + 1 ) {λ}^{1.5 k}} M + \frac{{r}^{3} M}{3 {λ}^{3} {h}^{3}} ( 0.3006 - f ( \frac{b}{h {λ}^{1.5}} ) ) \qquad ( 1 )$$

where r is the particle radius, a is the aspect ratio of the wavy chain structure, λ is the stretch in the compress direction, M is the magnetization of the micromagnets, k is a constant characterizing the influence of nonideality, neighboring chain-chain interaction, and macroscopic shape effect to the demagnetizing factor under compressive deformation, b and h are horizontal and vertical distances between the neighboring micromagnets (FIG. 84G), respectively, and 0.3006−f(x) is the dipole alignment factor, which describes the contribution of all other dipoles to the vertical magnetic field of a single dipole in the wavy chain. According to the wavy chain model, under external pressure, the compressed chain structure varies and alternates the dipole-dipole interaction inside the chain associated with the decrease in magnetic flux density. To verify the accuracy of the wavy chain model, embodiments marked possible chain structures observed in the soft magnetoelastic film (FIG. 84H) and measured the parameters b and h. Based on these parameters, embodiments estimated the magnetic flux density variation under the applied pressure, fitting well with the experiment observation (FIG. 84I).

The giant magnetoelasticity can generate the localized magnetic flux density change in response to the tiny pressure on the soft magnetoelastic film. Combined with a textile coil (FIG. 85A), this mechanical energy can be further converted into electricity by using the Faraday's law of induction:

$$ε = - N * \frac{d Φ}{dt} \qquad ( 2 )$$

where ε is the electromotive force (EMF), N is the turn of coil, Φ is the magnetic flux, and t is the time. In this case, a textile MEG was developed with a soft magnetoelastic film for magnetomechanical coupling and a textile coil for electromagnetic induction (FIG. 85B). The textile coil consists of the machine-sewn conducive yarns and a textile substrate (FIG. 85C), which can be easily produced by factory scale. Meanwhile, textile MEGs are fully waterproof without encapsulation because the magnetic fields can pass through fluid with negligible intensity loss (FIG. 85D). This distinct advantage allows textile MEGs to maintain their function even with heavy perspiration (FIG. 85E), holding wide practicability for wearable biomechanical-to electrical energy conversion. It is worth mentioning that the present textile MEG is fundamentally different from the reported wearable electromagnetic generators (Wan, J., Guo, H., Wang, H., Miao, L., Song, Y., Xu, C., Xiang, Z., Han, M., and Zhang, H. (2021). Magnetic, conductive textile for multipurpose protective clothing and hybrid energy harvesting. Appl. Phys. Lett. 118, 143901; Wan, J., Wang, H., Miao, L., Chen, X., Song, Y., Guo, H., Xu, C., Ren, Z., and Zhang, H. (2020). A flexible hybridized electromagnetic-triboelectric nanogenerator and its application for 3D trajectory sensing. Nano Energy 74, 104878; Rahman, M. T., Rana, S. S., Salauddin, M., Maharjan, P., Bhatta, T., and Park, J. Y. (2020). Biomechanical energy-driven hybridized generator as a universal portable power source for smart/wearable electronics. Adv. Energy Mater. 10, 1903663; Zhang, K., Wang, X., Yang, Y., and Wang, Z. L. (2015). Hybridized electromagnetic-triboelectric nanogenerator for scavenging biomechanical energy for sustainably powering wearable electronics. ACS Nano 9, 3521-3529): (1) Textile MEG is a one-body design that relies on the intrinsic magnetic flux density variation caused by the giant magnetoelasticity, while electromagnetic generators require the magnets moving relative to the circuits (FIG. 94). (2) Faraday's law is a single equation describing two different phenomena, the motional EMF generated by a magnetic force on a relatively moving circuits (electromagnetic generators) and the transformer EMF generated by an electric force due to a changing magnetic field (MEG).

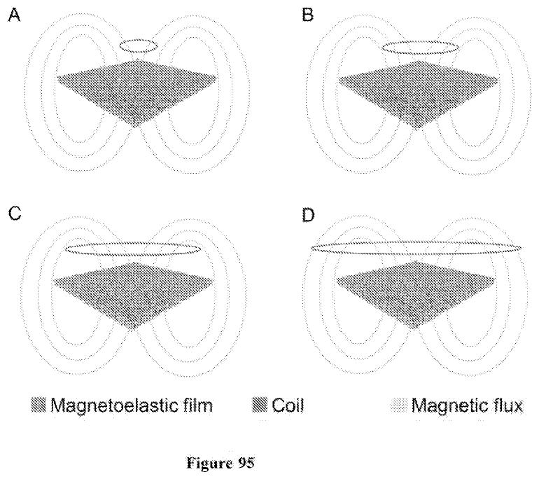

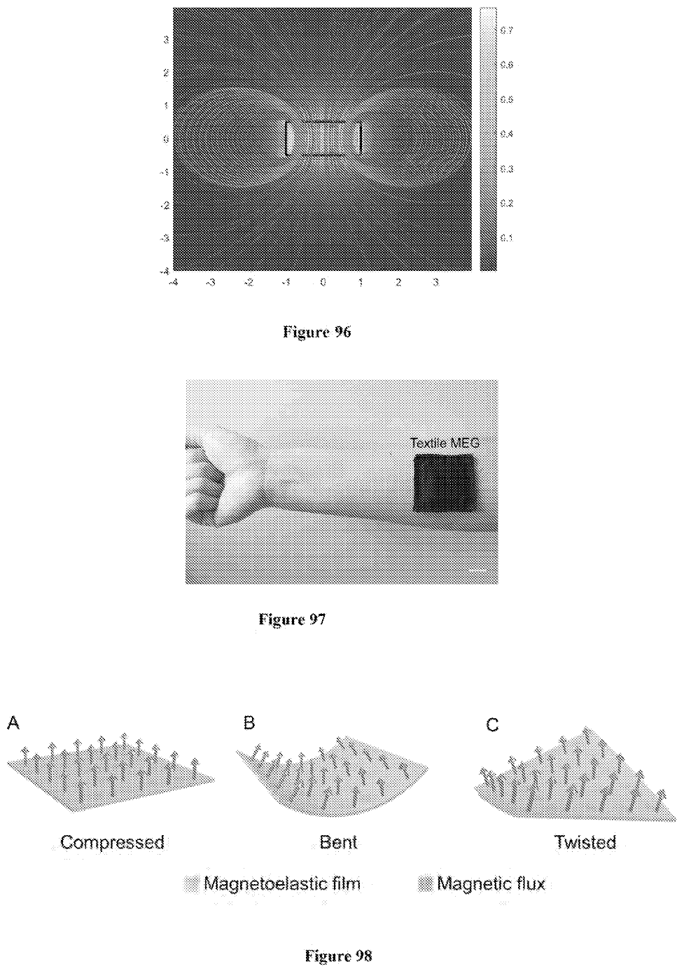

To optimize the biomechanical-to-electrical energy conversion of the textile MEG, systematically investigated was the interaction between the soft magnetoelastic film and the textile coil. First, the dependence of the electric output on the area ratio of the textile coil to the soft magnetoelastic film was plotted on FIG. 85F. A smaller textile coil (S_{coil}/S_{film}<1) cannot collect all magnetic flux density variation of the compressed soft magnetoelastic film, while a large textile coil (S_{coil}/S_{film}>1) consists of adverse magnetic field lines canceling each other out (FIG. 95). In this case, the approximately same size of the textile coil and the soft magnetoelastic film is the optimal ratio to biomechanical-to-electrical energy conversion. Secondly, regarding the distance between the two components, textile coil closely attached to the soft magnetoelastic film could generate the maximal electric outputs (FIG. 85G), since the strength of the magnetic field decreases with distance (FIG. 96). Finally, FIG. 85H clearly demonstrates that both output voltage and current show a linear relationship with the turns of textile coil, fitting well with the Faraday's law of induction (Equation 2). Thus, increasing the turns of textile coil is an effective method to promote the electric output. It is worth mentioning that the growth rate of output current is lower than that of output voltage because of the increased coil resistance.

### Textile MEGs for Energy Conversion

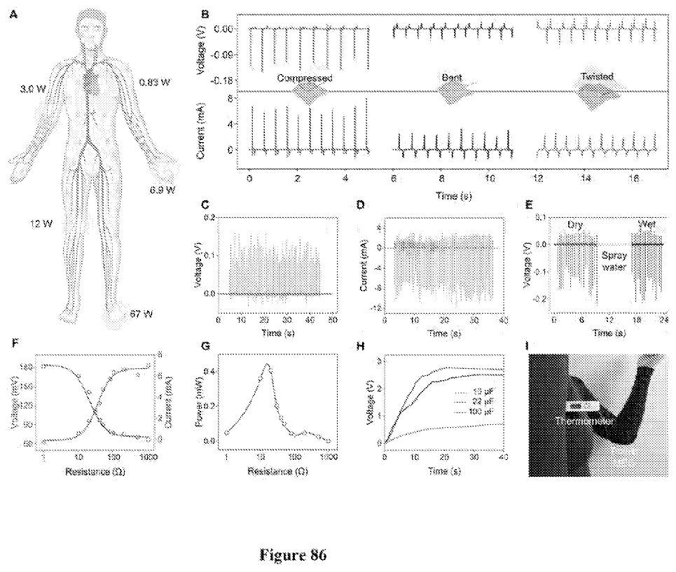

The human body is particularly rich in biomechanical energy, which can be explored as a pervasive power source for a wide range of wearable electronics such as sensors, actuators, and displays. Typical biomechanical activities such as the blood flow, breathing, upper limb movement, finger typing, and walking approximately contain a potential energy of 0.93 W, 0.83 W, 3.0 W, 6.9 mW, and 67 W, respectively (FIG. 86A). Commonly used biomechanical energy conversion technologies rely on triboelectric effect and piezoelectric effect-based nanogenerators. However, these nanogenerators have unavoidable disadvantages such as a very low current density in the order of 100 nA/cm² and a high internal impedance in the order of megaohms, which are caused by their capacitive power generation principle via manipulating the electric dipoles at the materials interfaces. Meanwhile, the generated current originates from the electric dipoles transfer at the materials interfaces, which is vulnerable to the perspiration and ambient humidity, severely limiting their functionality available to wearable applications. Additive encapsulation layer on these nanogenerators, such as silicone rubber (Wang, L., Liu, W., Yan, Z., Wang, F., and Wang, X. (2021). Stretchable and shapeadaptable triboelectric nanogenerator based on biocompatible liquid electrolyte for biomechanical energy harvesting and wearable human-machine interaction. Adv, Funct. Mater. 31, 2007221; Lan, L. Yin, T., Jiang, C., Li, X., Yao, Y., Wang, Z., Qu. S., Ye, Z., Ping, J., and Ying, Y. (2019). Highly conductive 1D-2D composite film for skin-mountable strain sensor and stretchable triboelectric nanogenerator. Nano Energy 62, 319-328; Wang, X., Yin, Y., Yi, F., Dai, K., Niu, S., Han, Y., Zhang, Y., and You, Z. (2017). Bioinspired stretchable triboelectric nanogenerator as energy-harvesting skin for self-powered electronics. Nano Energy 39, 429-436) have been developed for waterproofness. However, the thickness of the layer would impede the biomechanical energy transfer from the human body to the devices. In contrast, based on the coupling of the giant magnetoelasticity and Faraday's law of induction, textile MEGs feature a low internal resistance and can generate a high output current in the coil. Meanwhile, magnetic field variation can penetrate water with negligible intensity loss, endowing textile MEGs with intrinsic waterproof ability. These properties make textile MEGs highly competent in wearable energy conversion for powering bioelectronics.

To fulfill this duty, a textile MEG consisting of a textile coil with 200 turns was developed for wearable energy conversion. This textile MEG can be conformably attached to the human skin owing to its high flexibility (FIG. 97). To optimize the energy conversion performance, systematically investigated was the electric output of textile MEGs under different deformation modes.

The textile MEG was compressed, bent, and twisted to various shapes, and the corresponding voltage and current outputs were demonstrated in FIG. 86B. Compressed textile MEG performed best, because the vertically applied pressure can strongly decrease the magnetic flux density, while other deformation modes would partially cancel the magnetic field variation (FIG. 98).

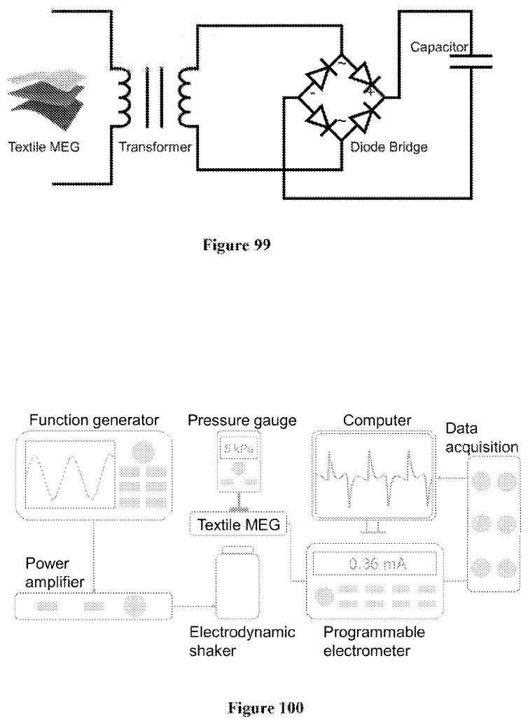

Meanwhile, under compression, the magnetic field variation is vertical to the coil, which could be fully utilized to generate a maximum EMF. Based on the compressive deformation, the textile MEG was continuously tapped by the human hand, generating an open-circuit voltage (Voc) of up to 198 mV (FIG. 86C) and a short-circuit current (Isc) of up to 12.4 mA (corresponding to a current density of 1.37 mA/cm²) (FIG. 86D). Moreover, the output voltage of the textile MEG remains constant even after being sprayed by water (FIG. 86E), demonstrating the ability for wearable energy conversion in the wet environment, such as the heavily perspiring body during exercise. To test the output power of the textile MEG, external resistors were loaded with the circuit. As the load resistance increased from 1 (2 to 1000 (2, the output current decreased and the output voltage increased (FIG. 86F). The maximum instantaneous power of 0.405 mW could be achieved when the load resistance was around 20Ω (FIG. 86G). With a rectifying circuit to convert the alternative output into the direct output (FIG. 99), the textile MEG charged capacitors of 10 μF, 22 μF, and 100 μF to 3 V, 3 V, and 1.1 V, respectively, within 20 seconds instantaneously by gentle touch (<5 N, 5 Hz) (FIG. 86H). The collected electricity in the capacitor could drive a commercial thermometer (FIG. 86I) for multiple physiological information monitoring.

### Textile MEG for Self-Powered Sensing

Physiological activities inside the human body generate various biomechanical signals (Zang, Y., Zhang, F., Di, C.-a., and Zhu, D. (2015). Advances of flexible pressure sensors toward artificial intelligence and health care applications. Mater. Horiz. 2, 140-156), such as pulse wave, blood flow, intestinal peristalsis, and many others. Self-powered biomechanical sensors can convert these biosignals into electrical signals, providing abundant healthcare information for clinical diagnosis. Typically applied mechanisms of these self-powered biomechanical sensors are triboelectric effect and piezoelectric effect, which rely on their capacitive electricity generation principle via manipulating the electric dipoles at the materials interfaces. However, this process is vulnerable to perspiration and ambient humidity from the human body, impeding their wearable applications. Although additive encapsulation layers could enhance their waterproofness, the thickness of the layer would impede biomechanical signals transferring from the human body to the devices, ultimately decreasing the sensitivity. In contrast, Textile MEGs are fully waterproof without encapsulation because the magnetic fields can pass through fluid with negligible intensity loss. Thus, textile MEGs can also work as self-powered biomechanical sensors for continuously monitoring human physiological signals, especially in the heavy perspiration situation such as exercising and under heat. Relevant biomechanical motions, such as the skin-surface fluctuation caused by arterial pressure and chest movement during breathing, can deform the textile MEGs and cause magnetic field distortion, inducing an EMF and generating the current in the textile coil. Meanwhile, textile MEGs feature outstanding wearability, such as breathability and softness, making them conformally attached to the human body for longterm monitoring.

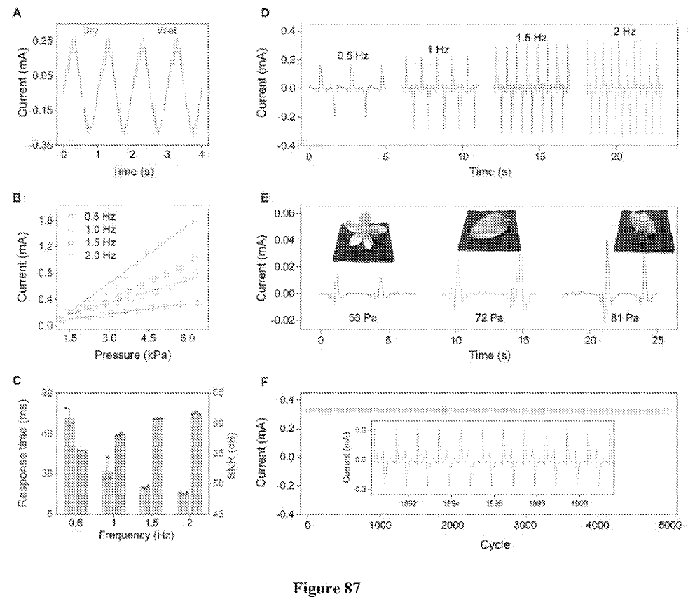

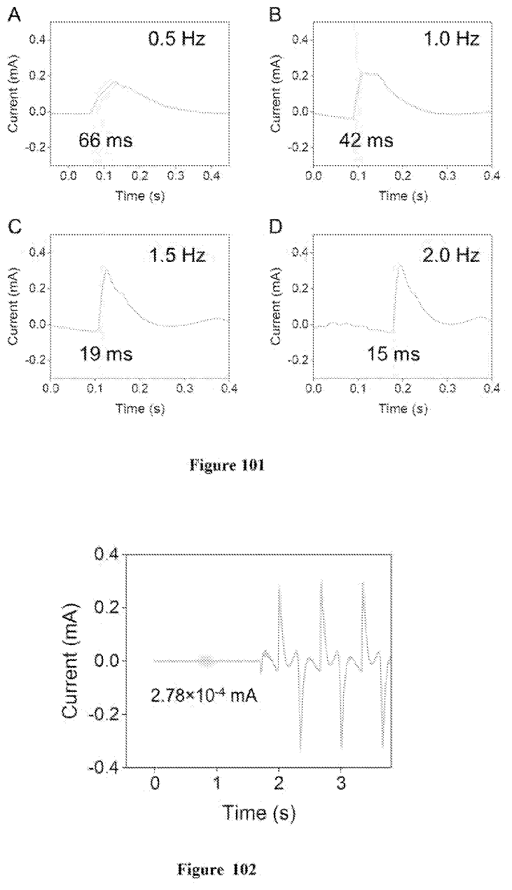

To characterize the sensing performance of textile MEGs in various aspects, established was a testing system containing a function generator, power amplifier, electrodynamic shaker, pressure gauge, programmable electrometer, and computer (FIG. 100). First, embodiments investigated the waterproofness of the textile MEGs during sensing. As shown in FIG. 87A, a textile MEG was deformed by a periodic gentle pressure of 1 Hz with sprayed water, while the acquired current signals are almost the same as the sensing signals from the dry situation. To further test the sensing performance, embodiments measured the sensitivity of the textile MEG by plotting current versus pressure characteristics under diverse frequencies. The testing system applied an external mechanical excitation of up to 6.5 kPa with a frequency of 0.5 Hz, 1.0 Hz, 1.5 Hz, and 2.0 Hz on the textile MEG. As shown in FIG. 87B, the output current of the textile MEG increases with the increased applied pressure as well as the increased frequency. Under a frequency of 2 Hz, the maximum sensitivity of 0.27 mA/kPa is realized. Then, embodiments investigated the sensing signal quality of the textile MEG in response to various frequencies. Under the fixed pressure of 2 kPa, the response time, signal-to-noise ratio (SNR), and pulse waveforms were measured, as shown in FIG. 87C and FIG. 87D. With an increased frequency, the output waveforms have a shorter response time (FIG. 101) and a higher SNR with a decreased standard deviation (FIG. 102). These results show that textile MEGs have a better sensing performance in response to the high frequency excitation, realizing a fast: response time (up to 15 ms), a high SNR (61.8 dB), and a stable output.

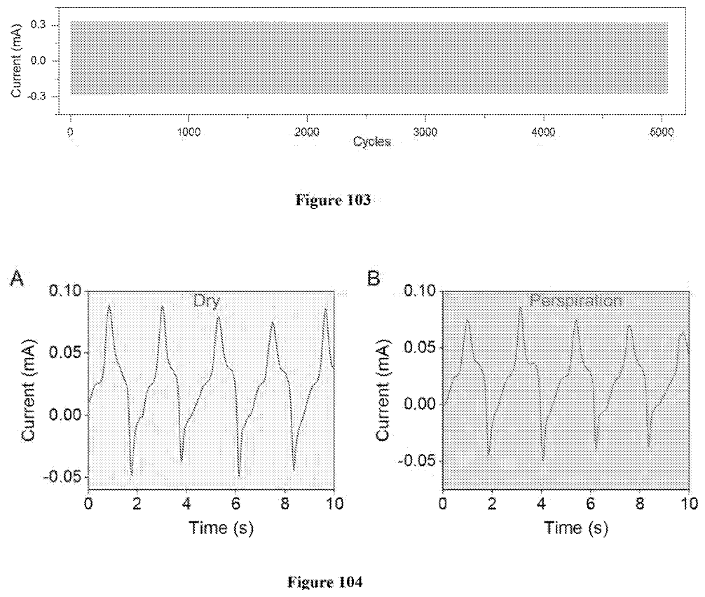

This phenomenon is attributed to the unique working mechanism of textile MEGs. According to Equation (2), the generated EMF is proportional to the variation speed of magnetic flux. Under the high frequency mechanical excitation, the magnetic field of the soft magnetoelastic film changes rapidly, inducing larger electric output signals with higher quality. This novel property enhances the ability of textile MEGs to distinguish the abnormal physiological signals with frequency variation, such as an increased heart rate and breathing rate. Further tested was the sensing performance of textile MEGs in the ultra-low-pressure range. A tiny white flower, a green leaf, and a yellow flower were gently dropped onto the textile MEG (Video S2). FIG. 87E shows the current signals of the textile MEG in response to the tiny subject loading. High sensitivity indicates that textile MEGs can detect a subtle pressure. Finally, to evaluate the stability of the textile MEG as a self-powered sensor, measured was the output current in response to loading unloading pressure cycles. An amplitude-fixed 2 kPa pressure with a frequency of 2 Hz was applied to the textile MEG for 5,000 loading-unloading cycles (FIG. 103). The peak-to-peak current was recorded (FIG. 87F), and the enlarged views of the current waveforms are shown in the inset. These results reveal a remarkable repeatability, stability, and durability of textile MEGs, proving their long-term monitoring ability.

### Machine Learning Assisted Respiratory Monitoring

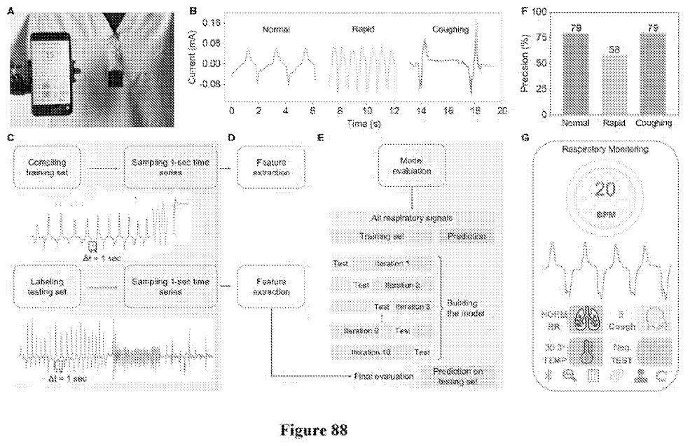

Respiration rate is one of the four main vital signs of the human body, providing critical personalized information for diagnosing respiratory diseases, such as pneumonia, asthma, and chronic bronchitis (Dinh, T., Nguyen, T., Phan, H.-P., Nguyen, N.-T., Dao, D. V., and Bell, J. (2020). Stretchable respiration sensors: Advanced designs and multifunctional platforms for wearable physiological monitoring. Biosens. Bioelectron. 166, 112460). Practically, respiration rate could be determined by measuring the rising and falling of the chest. Many wearable respiration sensors have been developed to convert these chest movements into electrical signals (Pegan, J. D., Zhang, J., Chu, M., Nguyen, T., Park, S.-J., Paul, A., Kim, J., Bachman, M., and Khine, M. (2016). Skin-mountable stretch sensor for wearable health monitoring. Nanoscale 8, 17295-17303; Zhao, Z., Yan, C., Liu, Z., Fu, X., Peng, L.-M., Hu, Y., and Zheng, Z. (2016). Machinewashable textile triboelectric nanogenerators for effective human respiratory monitoring through loom weaving of metallic yarns. Adv. Mater. 28, 10267-10274), creating a promising method for respiratory monitoring and personalized healthcare. However, human chest is one of the most common areas of sweating, featuring a high mean sweating rate of 1.555 mg·cm⁻² min⁻¹ (Baker, L. B., Ungaro, C. T., Sopeña, B. C., Nuccio, R. P., Reimel, A. J., Carter, J. M., Stofan, J. R., and Barnes, K. A. (2018). Body map of regional vs. Whole body sweating rate and sweat electrolyte concentrations in men and women during moderate exercise-heat stress. J. Appl. Physiol. 124, 1304-1318). Many wearable respiration sensors are intolerable to the humidity of sweat, and always need a bulky and airtight layer for encapsulation (Jeong, H., Rogers, J. A., and Xu, S. (2020). Continuous on-body sensing for the COVID-19 pandemic: Gaps and opportunities. Sci. Adv. 6, eabd4794), which might significantly reduce their sensitivity and wearing comfort. In contrast, textile MEGs are instinctive waterproof without any encapsulation, because the magnetic fields can pass through water with negligible intensity loss. Thus, the present textile MEGs can be seamlessly sewn on the clothes or chest strap with high air permeability and wearing comfort, working as a self-powered sensor for respiratory monitoring in the long term even with heavy perspiration (FIG. 88A).

To fulfill this duty, the textile MEG was directly stitched around the chest area of a nursing scrub. The respiration-caused expansion and contraction of the ribcage deform the textile MEG, generating a current output. Then tested was the sweatproof ability of the textile MEGs by spraying the artificial perspiration onto the device-embedded nursing scrub during the respiratory monitoring (FIG. 104). The acquired respiration signals are almost the same as the signals from the dry situation. These results indicate the excellent sweatproof ability of textile MEGs, which makes their feasible monitoring of respiratory activities. A 21-year-old man dressed in the textile MEG imitated three different kinds of respiratory patterns: normal breathing, rapid breathing, and coughing, respectively. The acquired waveforms are plotted in FIG. 88B. Due to the high sensitivity and stability of textile MEGs, the frequency, intensity, and persistency of different respiratory patterns were distinctly recorded. These data provide enough features for respiratory diseases assessment and diagnosis, which necessitate accurate and automated analysis and an infrastructure to enable quick medical intervention.

Machine learning is an emerging branch of data analysis that has shown early promises for personalized healthcare through the extraction of clinically relevant information from the imperceptible abnormal biosignals (Krittanawong, C., Rogers, A. J., Johnson, K. W., Wang, Z., Turakhia, M. P., Halperin, J. L., and Narayan, S. M. (2021). Integration of novel monitoring devices with machine learning technology for scalable cardiovascular management. Nat. Rev. Cardiol. 18, 75-91). On this basis, embodiments established a machine learning algorithm to classify different respiratory activities according to the present sensing signals. A laboratory-scale sensing dataset was collected by the present textile MEGs for the machine learning model training, which included more than 300 cycles of normal breathing, 400 cycles of rapid breathing, and 20 cycles of cough. First, these sensing signals were preprocessed by sampling 1-sec time series, generating the training records (FIG. 88C). Two types of feature extraction were used, including minimal features used to describe the data with the minimum number of features and efficient features that have larger number of features to describe the data (FIG. 88D). For both types of feature extraction, once the model extracted all features, another step was performed to keep only the relevant features. Two classifiers, decision tree and random forest, were used during the training process to learn these features. Ten-fold cross validation was used to evaluate machine learning models on these limited samples (FIG. 88E). Table S2 summarizes the average accuracy for cross validation. Random forest with efficient feature extraction gives the most accurate result of up to 90.89% as the cross validation mean accuracy. The testing sequence to evaluate the algorithm begins with 45-second normal breathing, 25-second rapid breathing, and 5 forced coughs. The present algorithm demonstrated the classification precision of 0.79, 0.58, and 0.79 for normal breath, rapid breath, and cough, respectively (FIG. 88F). Standard metrics of the algorithm performance, i.e., precision, recall, F-score, and overall accuracy, are summarized in Tables S3 & S4. This algorithm can be used to distinguish abnormal respiratory activities, such as rapid breathing and coughing from normal breathing, demonstrating the potential of respiratory diseases assessment, such as respiratory-based COVID-19 diagnoses.

TABLE S2

| | | | Number of | |
|---|---|---|---|---|
| Classifier | Features | Feature Type | features | Average Accuracy (%) |
| Decision | minimal | all | 9 | 73.61 |
| tree | minimal | selected/filtered | 5 | 72.79 |
| | efficient | all | 781 | 80.61 |
| | efficient | selected/filtered | 248 | 82.12 |
| Random | minimal | all | 9 | 79.45 |
| forest | minimal | selected/filtered | 5 | 78.62 |
| | efficient | all | 781 | 90.89 |
| | efficient | selected/filtered | 248 | 89.49 |

TABLE S3

| | Average | | | | | |
|---|---|---|---|---|---|---|
| Feature (Type) | Accuracy (%) | AUC | Mode | Precision | Recall | F-score |
| minimal (all) | 61.32 | 0.68 | normal | 0.71 | 0.65 | 0.68 |
| minimal (all) | | | rapid | 0.62 | 0.32 | 0.42 |
| minimal (all) | | | cough | 0.53 | 0.77 | 0.63 |
| minimal (filtered) | 58.49 | 0.66 | normal | 0.66 | 0.67 | 0.67 |
| minimal (filtered) | | | rapid | 0.53 | 0.32 | 0.4 |
| minimal (filtered) | | | cough | 0.52 | 0.66 | 0.58 |
| efficient (all) | 66.98 | 0.74 | normal | 0.8 | 0.72 | 0.76 |
| efficient (all) | | | rapid | 0.54 | 0.56 | 0.55 |
| efficient (all) | | | cough | 0.62 | 0.69 | 0.65 |
| efficient (filtered) | 34.91 | 0.50 | normal | 0.5 | 0.11 | 0.18 |
| efficient (filtered) | | | rapid | 0 | 0 | 0 |
| efficient (filtered) | | | cough | 0.33 | 0.91 | 0.49 |

TABLE S4

| | Average | | | | | |
|---|---|---|---|---|---|---|
| Feature (Type) | Accuracy (%) | AUC | Mode | Precision | Recall | F-score |
| minimal (all) | 63.21 | 0.78 | normal | 0.67 | 0.74 | 0.7 |
| minimal (all) | | | rapid | 0.32 | 0.38 | 0.47 |
| minimal (all) | | | cough | 0.66 | 0.71 | 0.68 |
| minimal (filtered) | 61.32 | 0.74 | normal | 0.64 | 0.74 | 0.69 |
| minimal (filtered) | | | rapid | 0.54 | 0.28 | 0.37 |
| minimal (filtered) | | | cough | 0.6 | 0.69 | 0.64 |
| efficient (all) | 73.58 | 0.82 | normal | 0.79 | 0.89 | 0.84 |
| efficient (all) | | | rapid | 0.58 | 0.6 | 0.59 |
| efficient (all) | | | cough | 0.79 | 0.63 | 0.7 |
| efficient (filtered) | 33.02 | 0.640 | normal | 0 | 0 | 0 |
| efficient (filtered) | | | rapid | 0 | 0 | 0 |
| efficient (filtered) | | | cough | 0.33 | 1 | 0.5 |

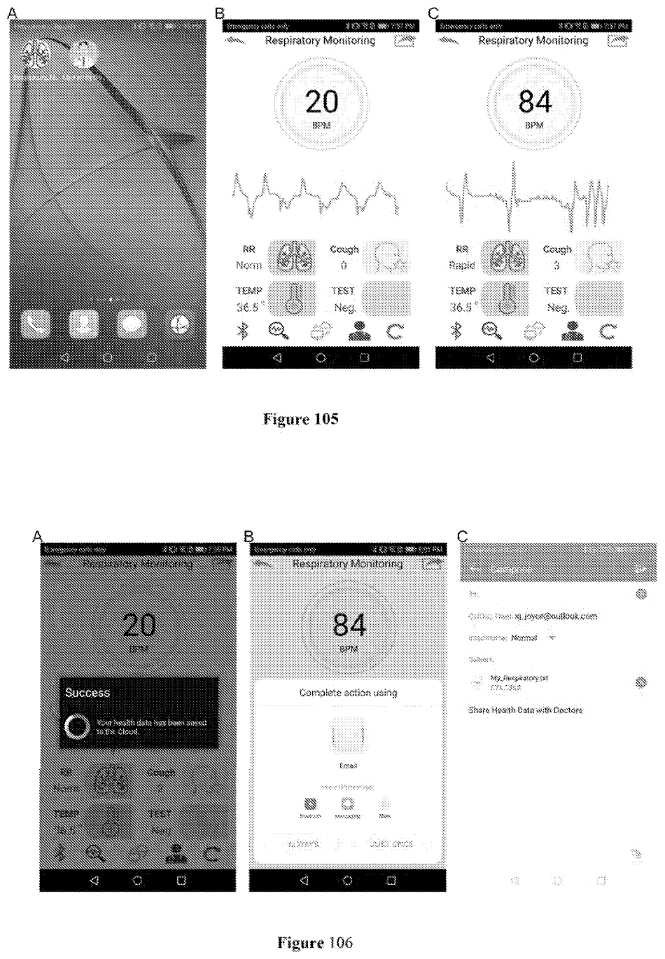

Further developed was a respiratory monitoring system including the textile MEGs, machine learning algorithms, and a customized cellphone application (APP) for data display, storage, and sharing. Respiratory monitoring signals acquired by textile MEGs were first amplified and filtered to obtain a high quality. Then the collected data was processed by the present machine learning algorithm to distinguish the respiratory patterns: normal, rapid, and coughing, and calculate the corresponding breaths per minute (BPM) and cough times. Finally, these data were transmitted to the cellphone APP and displayed in the front-ends (FIG. 88G and FIG. 105). All these patient-generated data can be one-click forwarded to physicians through email, cloud electronic health record, or message for further respiratory diseases diagnosis (FIG. 106). In brief, textile MEGs assisted by machine learning for respiratory monitoring can serve as a quantitative basis for detecting early signs of respiratory diseases in potential patient, monitoring the symptomatic progression in home settings, and tracking responses to therapeutics in clinical settings.

### D. Conclusions

The giant magnetoelasticity in soft matter can convert the applied pressure into enormous magnetic flux density change through the micromagnet interaction in the polymer matrix without externally applied magnetic field. To explore this phenomenon, a wavy chain analytical model was established to investigate the scientific principles and a textile MEG was invented to convert the biomechanical motions into electricity. Compared to the existing e-textile for biomechanical energy harvesting, the present textile MEGs feature intrinsic waterproofness, ultralow internal impedance, and high current density output.

From a scientific standpoint, Applicant discovered the giant magnetoelasticity in soft matter without the needs of externally applied magnetic field. It shows a magnetomechanical coupling factor of 6.77×10-8 T/Pa, which is up to five times larger than that of traditional rigid metal-based counterparts under magnetic field. Meanwhile, to understand the scientific principles of the giant magnetoelasticity, a wavy chain model based on the magnetic dipole-dipole interaction was established, fitting well to the experimental observation. From a material standpoint, the developed soft matter is flexible and stretchable, featuring a Young's modulus of 433.3 kPa and a stretchability of 350%. Comparing to the conventional rigid metal alloys with the Young's modulus of up to 100 GPa, the mechanical properties of the present soft matter can be more easily adapted by human skin and tissues. From an application standpoint, developed was a textile MEG by coupling the giant magnetoelasticity in soft matter with Faraday's law of induction, as a new mechanism for biomechanical-to-electrical energy conversion. The present textile MEGs demonstrated an ultralow internal impedance around ˜20Ω and a high short-circuit current density of 1.37 mA/cm², corresponding to four orders of magnitude enhancement than other textile counterparts for biomechanical energy conversion. Meanwhile, textile MEGs are intrinsic waterproof, which can also work as self-powered sensors for respiratory monitoring on a heavy perspiration skin. Assisted by machine learning, respiration abnormalities could be continuously and precisely detected, demonstrating the potential of respiratory diseases assessment. This collection of compelling features makes textile MEGs an emerging platform technology for the broad academic community.

In brief, the present study discovered the giant magnetoelasticity in soft matter consisting of a polymer matrix and micromagnets, which demonstrated a five times enhancement of magnetomechanical coupling factors than traditional rigid metal-based counterparts. To understand this phenomenon, a wavy chain analytical model based on the magnetic dipole-dipole interaction in the soft matter was established, fitting well to the experimental observation. Then explored was this discovery in e-textiles and coupled it with Faraday's law of induction to invent a textile MEG for biomechanical-to-electrical energy conversion. The developed textile MEG demonstrates an intrinsic waterproof property, an ultralow internal impedance, and a high current output.

Meanwhile, textile MEG can work as a self-powered sensor for respiratory monitoring on a heavy perspiration skin without any encapsulation. Assisted by machine learning, abnormal respiratory activities, such as rapid breathing and coughing can be precisely distinguished, demonstrating the potential of respiratory diseases diagnosis. It is believed that the discovery of giant magnetoelasticity can branch out into broader soft-matter systems, illuminating the future of e-textiles for developing human-body-centered energy, sensing, and therapeutic applications.

### D. Example Experimental Procedures

### Fabrication of the Soft Magnetoelastic Film

Ecoflex 00-30 part A (Smooth-on Inc.) and Ecoflex 00-30 part B (Smooth-on Inc.) with a weight ratio of 1:1 were mixed together and pre-cured in the room temperature for 10 minutes. Then neodymium-iron-boron micromagnets (MQFP-B-20076-088) with the weight concentrations of 40%, 60%, and 80% were blended with the polymer mixture by using a stirring rod. Stirring thoroughly for 10 minutes introduced air microbubbles for porous structure. Then the mixture was then cured at 70° C. in an oven (ThermoFisher) for 4 hours. Finally, the cured composited film was magnetized by an impulse field (approximately 2.6 T) using an impulse magnetizer (IM-10-30, ASC Scientific) to import the remnant magnetization.

### Characterization of the Soft Magnetoelastic Film

Structural characterization of the soft magnetoelastic film was conducted by SEM (Zeiss supra 40VP) and Micro-CT (CrumpCAT). Magnetic flux density mappings were realized by using a digital Gauss meter (TD8620, Tunkia) to continuously measure the surface magnetic field of the soft magnetoelastic film. Magnetic hysteresis loop was tested by a SQUID magnetometer (MPMS3, Quantum Design). The stress-strain curves were determined by using a dynamic mechanical analyzer (DMA, RSA III). The Young's modulus was calculated by fitting the experimental curves with a Neo-Hookean model (Deng, Z., and Dapino, M. J. (2018). Review of magnetostrictive materials for structural vibration control. Smart Mater. Struct. 27, 113001; Liu, J., Jiang, C., and Xu, H. (2012). Giant magnetostrictive materials. Sci. China Technol. Sci. 55, 1319-1326; Deng, Z. (2015). Nonlinear modeling and characterization of the villari effect and model-guided development of magnetostrictive energy harvesters and dampers. (The Ohio State University); Su, Q., Morillo, J., Wen, Y., and Wuttig, M. (1996). Young's modulus of amorphous terfenol-d thin films. J. Appl. Phys. 80, 3604-3606; Datta, S., Atulasimha, J., Mudivarthi, C., and Flatau, A. B. (2010). Stress and magnetic field-dependent young's modulus in single crystal iron gallium alloys. J. Magn. Magn. Mater. 322, 2135-2144).

### Fabrication of the Textile MEGs:

Flexible and thin conductive yarn (Remington Industries 43 HFVP.25) were sewn into the textile substrates by using a sewing machine (JUKI Automatic Industrial Sewing Machine) to construct the textile coil. These textile coils were stacked layer by layer and the conductive yarns from different layers were carefully connected end-to-end to construct a multilayered textile coil with different turns. Then a textile substrate, a the soft magnetoelastic film, and the textile coil with different turns and layers were stacked together to construct textile MEGs.

### Electrical Performance Characterization of the Textile MEGs

Electrical performance characterization system contained a function generator (AFG1062, Newark), power amplifier (PA-151, Labworks Inc.), electrodynamic shaker (ET-126HF, Labworks Inc.), and pressure meter (HYPX-017). Voltage signals were recorded by a programmable electrometer (Keithley 6514) and the current signals were recorded by a Stanford low-noise current pre-amplifier (Model SR570). For electricity generation, a diode bridge rectifier (MBSK16SE) was used to convert the alternative current to the direct current. A toroidal transformer was used to expand the voltage signals.

### Machine Learning Assisted Respiratory Patterns Recognition

For the laboratory-scale sensing dataset collection, a 21-year-old man dressed in the textile MEG performed 300 cycles of normal breath, 400 cycles of rapid breath, and 20 cycles of forced cough. Two classifiers, decision tree and random forest, were trained and tested. 10-fold cross validation was used for model training. Two types of feature extraction were used, including minimal features used to describe the data with the minimum number of features and efficient features that have a larger number of features to describe the data. For both types of feature extraction, once the model extracted all features, another step was performed to keep only the relevant features. Random forest with efficient feature extraction gave the most accurate results. Filtering the features, the accuracy decreased slightly due to less expensive computational costs. The performance of different classifiers was evaluated on the test set, consisting of 45-second normal breathing, 25-second rapid breath, and 5 forced coughs. The same feature extraction and feature types were applied on the test set as the training set.

### Mobile APP Design

The customized Android cellphone APP for data display, storage, and sharing was designed by using MIT A12 Companion. The respiratory patterns and respiration rate were acquired with the assistance of the present machine learning algorithms. Then these data were transmitted to the cellphone APP and displayed in the front-ends. The body temperature and the testing results were inputted by the users during the self-screening process.

### E: Stretchable, Inexpensive and Waterproof Magnetoelastic Sensor Array

### E. Summary

A stretchable, inexpensive, and waterproof magnetoelastic sensor array has been developed as a secondary skin for self-powered human machine interaction. The magnetoelastic sensor array utilizes the giant magnetoelastic effect in a soft system which converts mechanical pressure to magnetic field variation and when coupled with the magnetic induction, can generate electricity. In such a way, the present magnetoelastic sensor array comprises the giant magnetomechanical coupling layer made up of micromagnets and a porous silicone rubber matrix, and the magnetic induction layer, which are coils patterned by liquid metal. With programmable functionalities, the soft magnetoelastic sensor array can supply different commands by producing bespoke electric signals from human finger touch with an optimal signal-to-noise ratio of 34 dB and a rapid response time of 0.2 s. To pursue a practical application, the soft magnetoelastic sensor array can wirelessly turn on and off a household lamp and control a music speaker via Bluetooth continuously in real time, even with contact with high humidity environments such as heavy perspiration. With a collection of compelling features, the soft magnetoelastic sensor array puts forth a unique and savvy avenue of self-powered bioelectronic technology that practically enables a wider variety of applications wearable human-machine interaction.

Skin-integrated electronics that directly interact with machines are transforming our ways of life toward the emerging trend of the Metaverse. Consequently, developing a wearable and skin-conformal interface that simultaneously features waterproofness, low cost, and low power consumption for human-machine interaction remains highly desired. Herein, a stretchable, inexpensive, and waterproof magnetoelastic sensor array has been developed as a secondary skin for self-powered human machine interaction. The magnetoelastic sensor array utilizes the giant magnetoelastic effect in a soft system which converts mechanical pressure to magnetic field variation and when coupled with the magnetic induction, can generate electricity. In such a way, the present magnetoelastic sensor array comprises the giant magnetomechanical coupling layer made up of micromagnets and a porous silicone rubber matrix, and the magnetic induction layer, which are coils patterned by liquid metal. With programmable functionalities, the soft magnetoelastic sensor array can supply different commands by producing bespoke electric signals from human finger touch with an optimal signal-to-noise ratio of 34 dB and a rapid response time of 0.2 s. To pursue a practical application, the soft magnetoelastic sensor array can wirelessly turn on and off a household lamp and control a music speaker via Bluetooth continuously in real time, even with contact with high humidity environments such as heavy perspiration. With a collection of compelling features, the soft magnetoelastic sensor array puts forth a unique and savvy avenue of self-powered bioelectronic technology that practically enables a wider variety of applications wearable human machine interaction.

### E. Introduction

As the ever-growing presence of 5G infrastructure and the proliferation of the Internet of Things (IoT) become more robust, intelligent devices such as computers, machines, sensors, and many more, progressively provide human with more convenience (Z. Zhou, K. Chen, X. Li, S. Zhang, Y. Wu, Y. Zhou, K. Meng, C. Sun, Q. He, W. Fan, E. Fan, Z. Lin, X. Tan, W. Deng, J. Yang, and J. Chen, “Sign-to-speech translation using machine-learning-assisted stretchable sensor arrays,” Nat. Electron. 3, 571 (2020); Z. Yan, D. Xu, Z. Lin, P. Wang, B. Cao, H. Ren, F. Song, C. Wan, L. Wang, J. Zhou, X. Zhao, J. Chen, Y. Huang, and X. Duan, “Highly stretchable van der waals thin films for adaptable and breathable bioelectronic membranes,” Science 375, 852-859 (2022); A. Libanori, G. Chen, X. Zhao, Y. Zhou, and J. Chen, “Smart textiles for personalized healthcare,” Nat. Electron. 5, 142-156 (2022); G. Chen, Y. Li, M. Bick, and J. Chen, “Smart textiles for electricity generation,” Chem. Rev. 120, 3668-3720 (2020); G. Chen, X. Xiao, X. Zhao, T. Tat, M. Bick, and J. Chen, “Electronic textiles for wearable point-of-care systems,” Chem. Rev. 122, 3259-3291 (2022); G. Chen, Y. Fang, X. Zhao, T. Tat, and J. Chen, “Textiles for learning tactile interactions,” Nat. Electron, 4, 175 (2021); S. I. Rich, R. J. Wood, and C. Majidi, “Untethered soft robotics,” Nat. Electron. 1, 102-112 (2018)). Also, those technological examples are gradually transforming into more adaptive and intuitive, revolutionizing the bridge of communication between human and machines (W. Heng, S. Solomon, and W. Gao, “Flexible electronics and devices as human-machine interfaces for medical robotics,” Adv. Mater. 34, 2107902 (2021); X. Xiao, Y. Fang, X. Xiao, J. Xu, and J. Chen, “Machine-learning-aided self-powered assistive physical therapy devices,” ACS Nano 15, 18633 (2021). The global human-machine interface (HMI) market is expected to reach a value of 5.73 billion by 2023 at a compound annual growth rate of 9.37%. The growing desire for improved machines to monitor production and respond to the fast-changing demands, as well as the necessity for even higher efficiency and lower downtime, have fueled the expansion of the HMI market. This rapid growth suggests that now is an opportunistic time for the development of more innovative and creative approaches to connect human and machine even further; diversely ranging from hardware sensors to software algorithms.

On one hand, traditional HMIs require complex data collecting units and an enormous amount of power consumption, which call for external power sources that are bulky, rigid, environmentally unfriendly, and limited in lifetime. These disadvantages hinder HMI equipment the capabilities to transfer into practical and sustainable applications since it is nearly impossible to seamlessly incorporate wearable devices with conventional batteries while maintaining breathability and skin conformability, owing to the current designs in materials and volume. On this account, wearable HMI devices (R. Yin, D. Wang, S. Zhao, Z. Lou, and G. Shen, “Wearable sensors-enabled human-machine interaction systems: from design to application,” Adv. Funct. Mater, 31, 2008936 (2021); G. Chen, X. Zhao, S. Andalib, J. Xu, Y. Zhou, T. Tat, K. Lin, and J. Chen, “Discovering Giant Magnetoelasticity in Soft Matter for Electronics Textiles,” Matter 4, 3725-3740 (2021); X. Zhao, H. Askari, and J. Chen, “Nanogenerators for smart cities in the era of 5G and Internet of Things,” Joule 5, 1391 (2021)) with minimalistic features related to resistive effect (M. Amjadi, A. Pichitpajongkit, S. Lee, S. Ryu, and I. Park, “Highly stretchable and sensitive strain sensor based on silver nanowire-elastomer nanocomposite,” ACS Nano 8, 5154 (2014); C. Tan, Z. Dong, Y. Li, H. Zhao, X. Huang, Z. Zhou, J. W. Jiang, Y, Long, P. Jiang, T. Y. Zhang, and B. Sun, “A high performance wearable strain sensor with advanced thermal management for motion monitoring,” Nat. Commun. 11, 3530 (2020); S. Chen, Y. Song, D. Ding, Z. Ling, and F. Xu, “Flexible and anisotropic strain sensor based on carbonized crepe paper with aligned cellulose fibers,” Adv; Funct. Mater. 28, 1802547 (2018); Y. Yu, J. Nassar, C. Xu, J. Min, Y. Yang, A. Dai, R. Doshi, A. Huang, Y. Song, R. Gehlhar, A. D. Ames, and W. Gao, “Biofuel-powered soft electronic skin with multiplexed and wireless sensing for human-machine interfaces,” Sci. Robot. 5, 1 (2020)), capacitive effect (J. Lee, H. Kwon, J. Seo, S. Shin, J. H. Koo, C. Pang, S. Son, J. H. Kim, Y. H., Jang, D. E. Kim, and T. Lee, “Conductive fiber-based ultrasensitive textile pressure sensor for wearable electronics,” Adv. Mater. 27, 2433 (2015); Y. C. Huang, Y. Liu, C, Ma, H. C, Cheng, Q. He, H. Wu, C. Wang, C. Y. Lin, Y. Huang, and X. Duan, “Sensitive pressure sensors based on conductive microstructured air-gap gates and two-dimensional semiconductor transistors,” Nat. Electron. 3, 59 (2020); Y. Xiong, Y. Shen, L. Tian, Y. Hu, P. Zhu, R. Sun, and C. P. Wong, “A flexible, ultra-highly sensitive and stable capacitive pressure sensor with convex microarrays for motion and health monitoring,” Nano Energy 70, 104436 (2020)) and self-powered mechanisms based on triboelectric effect (G. Conta, A. Libanori, T. Tat, G. Chen, and J. Chen, “Triboelectric nanogenerators for therapeutic electrical stimulation,” Adv. Mater. 33, 2007502 (2021); K. Meng, S. Zhao, Y. Zhou, Y. Wu, S. Zhang, Q. He, X. Wang, Z. Zhou, W. Fan, X. Tan, J. Yang, and J. Chen, “A wireless textile-based sensor system for self-powered personalized health care,” Matter 2, 896 (2020); S. Zhang, M. Bick, X. Xiao, G. Chen, A. Nashalian, and J. Chen, “Leveraging triboelectric nanogenerators for bioengineering,” Matter 4, 845 (2021); Z. Lin, J. Chen, X. Li, Z. Zhou, K. Meng, W. Wei, J. Yang, and Z. L. Wang, “Triboelectric nanogenerator enabled body sensor network for self-powered human heart-rate monitoring,” ACS Nano 11, 8830 (2017); Y. Fang, Y. Zou, J. Xu, G. Chen, Y. Zhou, W. Deng, X. Zhao, M. Roustaei, T. K. Hsiai, and J. Chen, “Ambulatory cardiovascular monitoring via a machine-learning-assisted textile triboelectric sensor,” Adv. Mater. 33, 2104178 (2021)) and piezoelectric effect (P.-K. Yang, S.-A. Chou, C.-H. Hsu, R. J. Mathew, K.-H. Chiang, J.-Y, Yang, and Y,-T. Chen, “Tin disulfide piezoelectric nanogenerators for biomechanical energy harvesting and intelligent human-robot interface applications,” Nano Energy 75, 104879 (2020); Y. Su, C. Chen, H. Pan, Y, Yang, G. Chen, X. Zhao, W. Li, Q. Gong, G. Xie, Y. Zhou, S. Zhang, H. Tai, Y. Jiang, and J. Chen, “Muscle fibers inspired high-performance piezoelectric textiles for wearable physiological monitoring,” Adv. Funct. Mater. 31, 2010962 (2021); D. Zhang, D. Wang, Z. Xu, X. Zhang, Y. Yang, J. Guo, B. Zhang, and W. Zhao, “Diversiform sensors and sensing systems driven by triboelectric and piezoelectric nanogenerators,” Coord. Chem. Rev. 427, 213597 (2021)), or hybridized systems (G. Tang, Q. Shi, Z. Zhang, T. He, Z. Sun, and C. Lee, “Hybridized wearable patch as a multi-parameter and multi-functional human-machine interface,” Nano Energy 81, 105582 (2021); B. Zhang, J. Chen, L. Jin, W. Deng, L. Zhang, H. Zhang, M. Zhu, W. Yang, and Z. L. Wang, “Rotating-disk-based hybridized electromagnetic-triboelectric nanogenerator for sustainably powering wireless traffic volume sensors,” ACS Nano 10, 6241 (2016); P. Jiao, “Emerging artificial intelligence in piezoelectric and triboelectric nanogenerators,” Nano Energy 88, 106227 (2021)) have emerged to provide the current state-of-the-art technologies, especially those with energy-harvesting strategies for sustainable and environmentally friendly power generation by means of biomechanical motions. Despite the tremendous list of advantages, these working principles can still be vulnerable to humidity and deteriorate in liquid conditions (L. Li, X. Wang, P. Zhu, H. Li, F. Wang, and J. Wu, “The electron transfer mechanism between metal and amorphous polymers in humidity environment for triboelectric nanogenerator,” Nano Energy 70, 104476 (2020)), which limit their electrical outputs and applications in certain environments such as heavily perspiring exercises or usages in extreme weather.

On the other hand, the magnetoelastic effect is usually observed in rigid bulky alloys in nature (X. Zhao, Y. Zhou, J. Xu, G. Chen, Y. Fang, T. Tat, X. Xiao, Y, Song, S. Li, and J. Chen, “Soft fibers with magnetoelasticity for wearable electronics,” Nat. Commun. 12, 6755 (2021); K. Zeng, S. C. Roy, and C. A. Grimes, “Quantification of blood clotting kinetics I: determination of activated clotting times as a function of heparin concentration using magnetoelastic sensors,” Sens. Lett. 5, 425 (2007); S. C. Roy, K. G. Ong, K. Zeng, and C. A. Grimes, “Quantification of blood clotting kinetics II: thromboelastograph analysis and measurement of erythrocyte sedimentation rate using magnetoelastic sensors,” Sens. Lett. 5, 432 (2007); C. A. Grimes, S. C. Roy, S. Rani, and Q. Cai, “Theory, instrumentation and applications of magnetoelastic resonance sensors: a review,” Sensors 11, 2809 (2011)). Very recently, the present Applicant discovered the giant magnetoelastic effect in a soft materials system with up to four times enhancement than the traditional rigid counterpart (Y. Zhou, X. Zhao, J. Xu, Y. Fang, G. Chen, Y. Song, S. Li, and J. Chen, “Giant magnetoelastic effect in soft systems for bioelectronics,” Nat. Mater. 20, 1670 (2021)). In this work, the discovered giant magnetoelastic effect is employed to develop a programmable and waterproof sensor array for self-powered HMI. Each magnetoelastic sensing unit is revolutionarily conditioned with a characteristic output signal in order to correlate with programmable functionalities in controlling a machine. This unique feature comes from the programmed orientation of the magnetoelastic film during the initial magnetization process. The device demonstrates a strain up to 150%, a wide pressure sensitivity ranging from 10 kPa to 80 kPa, an optimal signal-to-noise ratio (SNR) of 34 dB, and a rapid response time of 0.2 s at the frequency of 1 Hz. The programmable magnetoelastic sensor array can produce continuously responsive electric signals and productively command electronic devices real-time via touch sensing of finger tapping. Importantly, it is intrinsically waterproof since the magnetic field could penetrate the water without much loss. To pursue a practical application, this device is integrated with a customized circuit system to portray as the on and off buttons for a desk lamp and function as four command features: play, pause, next, and previous, to control a music speaker. At the front end, the programmable magnetoelastic sensor array is capable of becoming a key player in the HMI communities whose future may require a self-powered, skin-conformal, flexible, stretchable, and waterproof innovations.

### E. Results and Discussion

### Structural Design and Working Principle

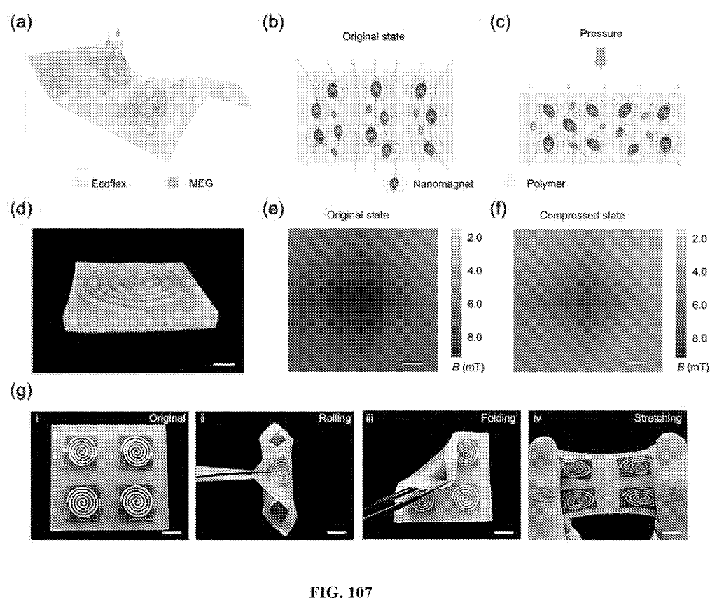

A 40-mm-by-40-mm programmable magnetoelastic sensor array, consisting of four sensors, is illustrated in FIG. 107(a) with a waterproof all-in-one body design. Each sensor mainly holds two functional components. One is the giant magnetomechanical coupling (MC) layer that comprises the solid neodymium-iron-boron (NdFeB) micromagnets and microbubbles-introduced porous silicone rubber matrix, which is able to convert the gentle biomechanical pressure into magnetic flux variation. The scanning electron microscope (SEM) image of the MC layer is displayed in the supplementary material, showing the scattered micromagnets and porous structure. The other functional component is the magnetic induction (MI) layer, which are the patterned liquid metal coils. A photograph of the liquid metal before patterning is shown in the supplementary material. The MI layer is responsible to pick up the magnetic field variation and generate electricity on the basis of electromagnetic induction.

The magnetoelastic sensor itself could convert biomechanical activities into electrical signals by using a two-step conversion process: the MC layer is responsible for the mechanical-to-magnetic conversion and the MI layer the magnetic-to-electrical conversion. As illustrated in FIG. 107(b), after magnetization and in the initial state, the micromagnets are single magnetic dipoles and aligned in a wavy chain structure. When each magnetoelastic sensor receives an applied uniaxial pressure, as shown in FIG. 107(c), the micromagnet chain structure diverges and internally alters the dipole-dipole interaction of the chain (shown in the supplementary material). The demagnetizing fields are proportional with the decrease in the surface magnetic flux density. Once the uniaxial stress is released, the recovery of the micromagnet wavy chain structure reverses the magnetic flux density back to its original state. The magnetoelastic effect in the magnetoelastic sensor is observed without the necessity of an external magnetic field. The micro-computed tomography (Micro-CT) images in FIG. 107(d) and in the supplementary material reveal that these micromagnets are evenly distributed and scattered throughout the porous matrix. FIG. 107(e) shows the shift in the magnetic flux density mappings of one magnetoelastic sensor. As illustrated in FIG. 107(f), under an applied pressure of 300 kPa, the magnetic flux density declines to about 50%. Owing to the materials' flexibility and durability, the magnetoelastic sensor array can also generate stable power under deformations, rolling, folding, and stretching as in FIG. 107(g). And due to these compelling features, the device can be adopted for human-body powered HMI by transforming human biomechanical activities into electrical signals

### Device Optimization

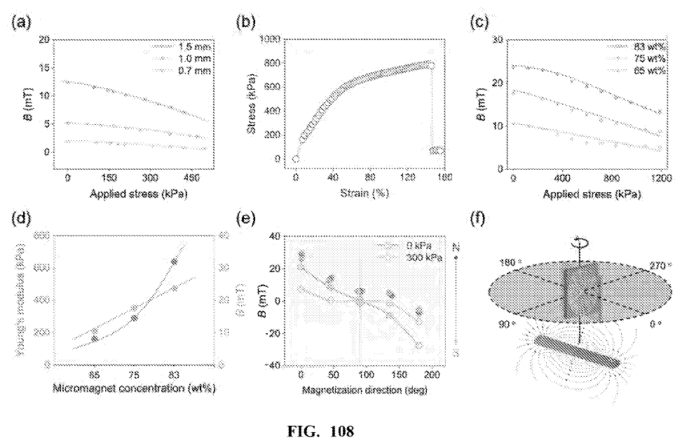

To optimize the biomechanical-to-electrical energy conversion of each individual magnetoelastic sensor, embodiments comprehensively investigate the assembly and properties of the soft magnetoelastic composite. First, by controlling the thicknesses and the magnetic particle concentration, the soft magnetoelastic composite shows different mechanical properties. The thickness to produce an optimal electrical output is plotted according to FIG. 108(a). Accordingly, thicker magnetoelastic composite provides higher magnetic flux. The thickness of 1.5 mm is chosen because it provides high magnetic field variation while still seemingly appears thin enough to exhibit flexibility, stretchability, and deformation. Second, as illustrated in FIG. 108(b), the 83 wt % soft magnetoelastic film is stretchable up to 150% strain. Since its decrease in magnetic flux density can compete to that of the traditional magnetoelastic system, 37 which needs an enormous amount of uniaxial stress of more than 10 MPa, embodiments proceed to sample different micromagnet concentrations to examine a variety of magnetic flux density alterations as appeared in FIG. 108(c). Under a continuous uniaxial applied stress, 83 wt % micromagnet concentration demonstrates the highest values of magnetic field variation of 10.3 mT more than those with 75 wt % (8.9 mT) and 67 wt % (5.2 mT) of micromagnets. The higher increase in micromagnet concentrations would be more difficult to combine with the polymer matrix and consequently, to form a flexible, stretchable, and deformable sensor. Therefore, 83 wt % is adequate enough to provide extensive output signals while still keeping the desired properties of the present magnetoelastic sensor array. In addition, the Young's modulus and the initial magnetic field strength of the magnetoelastic composite (10 mm×10 mm×1.5 mm), with different micromagnet concentrations, are measured, as shown in FIG. 108(d). Increasing in the concentration of the micromagnets not only could intensify the initial magnetic field but also raise the Young's modulus of the magnetoelastic system. The rearrangement of the micromagnets in the composite could possibly decrease the remanent magnetization and the coercive field in the compressed state, resulting in a negative fluctuation in the magnetic flux density. Furthermore, embodiments verify the magnetic field variation of the device in different magnetization angles under the original state and the applied pressure of 300 kPa. As evidenced in FIG. 108(e), the orientation that directly applies magnetization on the south and north direction provide the highest values of magnetic field variation, where north is the positive direction.

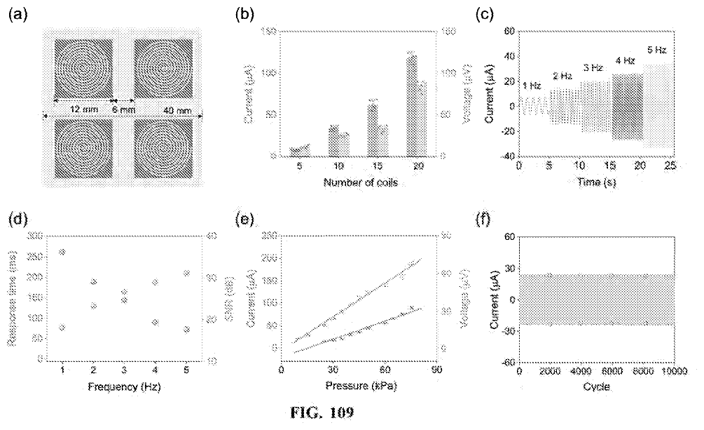

FIG. 108(f) displays the systematic configuration of how the magnetoelastic composite was oriented to conditioned different magnetization so each sensor can perform different commands. Subsequently, each soft magnetoelastic film is patterned with 20 turns of liquid metal coils to establish a fully completed magnetoelastic sensor array which include four different magnetoelastic sensors as shown in FIG. 109(a). In the present case, the proposed dimension allows the device to fit onto a human hand or wrist for HMI. However, the sensor array can be miniaturized for various application scenarios. The excellent composition of the materials gives great freedom for versatile sensor designs, including size, thickness, softness, and so on. To incorporate the magnetoelastic effect with the coil's electromagnetic induction, different numbers of coil turns are examined as a way to verify both the performance of the current and voltage outputs against the coil size. Consequently, FIG. 109(b) shows a well-behaved linear relationship. This is consistent with Faraday's law of induction which declares that both the number of liquid metal turns and the magnetic field variation of the MC layer are positively proportional to the electrical outputs. Consequently, 20 turns of coil are chosen due to their indication of high output signals while still equipping the magnetoelastic sensor array with the abilities to be skin-conformal, flexible, stretchable, and deformable. On the one hand, to characterize the electrical performance of the magnetoelastic sensor array, investigated were the pulse waveforms under different applied frequencies at a fixed pressure, as illustrated in FIG. 109(c) and the supplementary material and observe that increasing the applied frequencies yields higher electrical outputs. This result correlates to the working principle that the faster the magnetic field changes (the higher frequency), the greater the output electrical signal will be. On top of that, according to FIG. 109(d), with an increased frequency, the output signals exhibit a shorter response time and higher signal-to-noise ratio (SNR). The background noise is within a controllable range in the lab environment. Additionally, the magnetoelastic sensor array is sensitive enough to detect pressure in the range of human finger tapping. As shown in FIG. 109(e), both the current and voltage response linearly with an increase in pressure ranging from 10 kPa to 80 kPa, confirming a superb sensitivity in the range of human finger tapping. Meanwhile, 10,000 cycles of constant applied pressure were exerted onto the device to validate its durability. FIG. 109(f) substantiates the device's excellent stability and repeatability. The fast response time of 0.2 s at a frequency of 1 Hz, a favorable SNR of 34 dB, and a stable output performance reveal that the magnetoelastic sensor array performs better under high-frequency excitation and expresses significant stability and durability, which can be incorporated toward many long-term HMI applications.

### Self-Powered Human-Machine Interaction

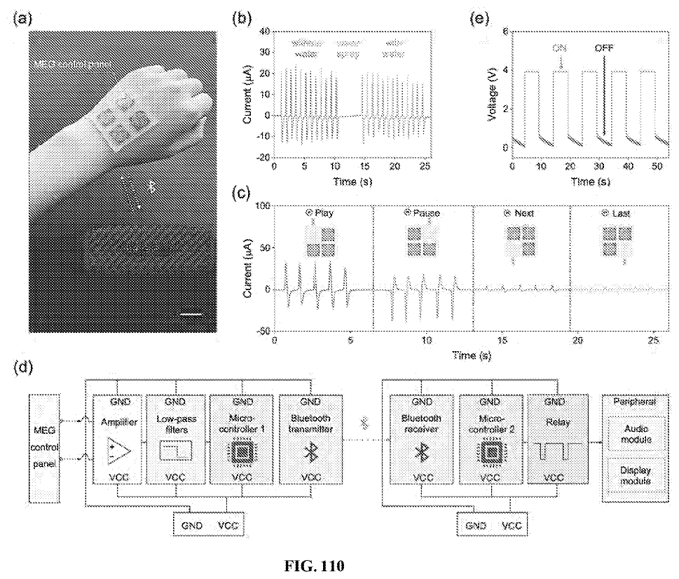

With the fundamentally new working principle, the array system is a promising design in applications of HMI for its wearability, flexibility, skin conformity, and stable electrical performance under the exposure to humid environment and submergence in water as illustrated in FIG. 110(a) and the supplementary material. The whole system is tested underwater, as shown in FIG. 110(b) and the supplementary material, to examine its performance in producing adequate energy in a liquid environment, such as accessing to control the equipment in a shower, in extreme weather, or under a heavily perspiring body during exercise. Notably, to validate the performance of the magnetoelastic sensor array as a Bluetooth wireless controller, four similar-in-appearance magnetoelastic sensors are embedded into a structural Ecoflex elastomer. Each is individually magnetized in different orientation to exhibit characteristic output signals in order to separately command the features: play, pause, next, and previous, of a music speaker, as shown in FIG. 110(c). To further explore the possibility of differentiating the control sensors, four participants are requested to touch each key. These results deliver output differences between each sensor but similarities between each subject. Thus, when a user is tapping on the magnetoelastic sensor array, the signal detected is conditioned, transmitted, and then converted into on and off signal for the electrical appliances (in the supplementary material). To integrate this technology with a commercialized music speaker as a part of the HMI application and confirm its competence in enacting wireless communication, a process flow system of the circuit system is developed, consisting of three components: a sensor array (four magnetoelastic sensors connected in parallel), a transmitter unit, and a receiver unit, as shown in FIG. 110(d). For the transmitter unit, the magnetoelastic sensor array directly collects the user's biomechanical finger tapping data. The hand gesture signals will then be collected and transferred to an analog circuit for careful amplification and filtration. This step ensures that the output signal can precisely express adequate details that are suitable for processing by an analog-to-digital converter (ADC) and further incorporating in commanding a third-party equipment. Additionally, the data would further be processed by a microcontroller before wirelessly being delivered from a Bluetooth module to another one at the receiver end. In this way, the second microcontroller can receive the command signals generated from the magnetoelastic sensor array, which can also precisely control the audio and display module inside the music speaker. Simultaneously, a latching relay is connected in series with the electrical appliances (music player, lamp, or fan). FIG. 110(e) shows the command signals collected after converting by the relay and in this way the magnetoelastic sensor array can successfully control a commercial music speaker.

### E. Conclusions

Enabled by the new discovery of magnetoelastic effect in the soft polymer system, a self-powered sensor array is developed for human-machine interaction with decent wearability and water resistance. It can effectively convert biomechanical signals from finger tapping into bespoke output signals to connect with desired machines. By integrating with a signal-processing circuit that includes an amplifier, the low-pass filters, the micro-controllers, the Bluetooth modules, a relay, and an audio and display module, the magnetoelastic sensor array not only wirelessly simulates as the on and off buttons of a lamp but also portrays as a music player's command features, representing the actions of play, pause, next, and previous. These applications are accomplished by the unique magnetization design of each magnetoelastic sensor in order for it to produce identifiable electrical signals. Importantly, between different users, these four output signals remain similar. The device exhibits a well-behaved linear variation in the forms of output voltage and current that show a superior sensitivity of 80 kPa, which is suitable for touching sensing, an optimal SNR of 34 dB, and a rapid response time of 0.2 s at 1 Hz. This work demonstrates a unique and compelling approach for self-powered bioelectronics and promises a great adaptable and versatile solution for users in water-resistant HMI applications to control their third-party machine anytime anywhere, ultimately improving our way of living in the smart generation of the IoT and 5G technologies.

### E. Example Experimental Methods

### Fabrication of the Multifunctional Magnetoelastic Sensor Array

All the soft MC layers are fabricated using Ecoflex 00-30 part A and Ecoflex 00-30 part B with a weight ratio of 1:1. Then, neodymium-iron-boron micromagnets (MQFP-B-20076-088) with weight concentrations of 65%, 75%, and 83% are combined with the polymer mixture using a stirring rod. Stirring thoroughly for 10 min introduced air microbubbles to produce desirable porous structure. Then the mixture is poured into a 3D printed template and cured at 70° C. in the oven (Thermo Fisher Scientific) for 4 h. By using different templates, composited films with given thickness could be fabricated. Finally, the cured composited film, positioned at 0, 45, 180 and 225∘ angle, is individually magnetized by an impulse field (approximately 2.6 T) using an impulse magnetizer (IM-10-30, ASC Scientific) to introduce different remnant magnetization patterns.

Ga (99.99%) and In (99.99%) ingots were purchased from RotoMetals to assemble the liquid metal. Eutectic gallium indium (EGaIn; 74.5 wt % Ga and 25.5 wt % In) is heated in a muffle furnace (ThermoFisher) at 200° C. for 2 h. Then, the liquid metal is mixed with 10 wt % Ni particles (99.5%, 5 μm, US Research Nanomaterials) thoroughly using a VWR mini Vortexer to acquire the desired rheological property as a way to improve processability before any usage. A laser cutting machine (ULTRA R5000, Universal Laser System) is used to cut a polyethylene terephthalate (PET) film in the shape of a square helix (length, 12 mm; width, 12 mm). The liquid metal is then patterned onto the soft magnetoelastic film using the PET film mask.

### Characterization of the Soft Magnetoelastic Film

Structural characterization of the soft magnetoelastic film was conducted by SEM (Zeiss supra 40VP) and micro-CT (CrumpCAT). Magnetic flux density measurement is succeeded using a digital Gauss meter (TD8620, Tunkia). Uniaxial stress is applied on the soft magnetoelastic film, and the Gauss meter with an axial probe measures the vertical component of the magnetic field. The stress-strain curves are determined by using a dynamic mechanical analyzer (DMA, RSA III). The Young's modulus is calculated by fitting the experimental curves with a neo-Hookean model.

### Characterization of the Magnetoelastic Sensor Array's Electrical Performance

The voltage signals of the Magnetoelastic sensors are measured by a Stanford low-noise voltage pre-amplifier (Model SR560) and current signals a Stanford low-noise current pre-amplifier (Model SR570). Real-time data acquisition and display are realized using the LabVIEW software. The stability of the magnetoelastic sensor is validated by a calibration electrodynamic transducer (Labworks, ET-126HF) at 20 Hz. The electrical output performance of the magnetoelastic sensor is measured at the different frequencies and applied forces. Finally, the pressure meter (HYPX-017) is used to apply an adjustable pressure to the magnetoelastic sensor.

### Circuit Design

The magnetoelastic sensor array and interaction system are composed of three parts, including a magnetoelastic sensor array, an integrated signal conditioning circuit (transmitter unit), and an integrated command control circuit (receiver unit). First, the electrical signals from the finger tapping are acquired from the magnetoelastic sensor array. The signals are then amplified and filtered by an analog circuit to remove environmental noise. Then, the analog signals are converted to digital signals by a microcontroller (Arduino UNO) and then transmitted wirelessly to the receiver unit through a Bluetooth module (HC-05). Another: Bluetooth module (HC-05) in the receiver unit receives these signals and passes them to a second microcontroller (Arduino UNO). Finally, a latching relay is connected and transforms the signals to different commands which can precisely control the audio and display module inside a music player as well as the on and off function of a lamp.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

1. An apparatus comprising:
a soft system with a giant magnetoelastic effect; and
a magnetic induction coupled to the soft system to implement a soft magnetoelastic generator (MEG).

2. The apparatus of claim 1, wherein the MEG comprises a textile MEG.

3. The apparatus of claim 1, wherein the MEG comprises a human-wearable MEG.

4. The apparatus of claim 3, wherein the human-wearable MEG is configured to convert an arterial pulse into electrical signals under the circumstance of heavy body perspiration for self-powered cardiovascular parameter measurement.

5. The apparatus of claim 4, further including a customized cellphone application configured to communicate with the human-wearable MEG.

6. The apparatus of claim 2, wherein the textile MEG has an intrinsic waterproof property, an ultralow internal impedance around ˜20Ω, and a high short-circuit current density of 1.37 mA/cm².

7. The apparatus of claim 2, wherein the textile MEG is configured as a self-powered textile respiration sensor.

8. The apparatus of claim 1, further comprising a stretchable and waterproof magnetoelastic sensor array for self-powered human-machine interaction.

9. The apparatus of claim 8, wherein the magnetoelastic sensor array comprises a giant magnetomechanical coupling layer including micromagnets and a porous silicone rubber matrix.

10. The apparatus of claim 9, wherein the magnetic induction comprises coils patterned by liquid metal.

11. The apparatus of claim 1, wherein the soft system is comprised of platinum-catalyzed silicone polymer matrix and neodymium-iron-boron nanomagnets.

12. The apparatus of claim 1, wherein the soft system comprises an elastic silicone microfiber.

13. The apparatus of claim 12, wherein the elastic silicone microfiber having an elastic hollow channel filled with a liquid metal alloy.

14. The apparatus of claim 13, wherein the liquid metal alloy comprises 74.5% Ga and 25.5% In by weight.
